SEQUENCE LISTING (86 sequences)

SEQ ID NO: 1 - protein, 736 aa, synthetic construct
  REGION 1..736: note = adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 736 aa, synthetic construct
  REGION 1..736: note = AAV isolate
MTADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKQLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTGGNFHPSPLMGGFGMKHPPPQILIKNTPVPADPPTAFNKDKLNSFITQYSTGQVSVEIEWELQKENSKRWNPEIQYTSNYYKSNNVEFAVNTGGVYSEPRPIGTRYLTRNL

SEQ ID NO: 4 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPINAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTYGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIDKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVLQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGSSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQARAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAVDAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPREPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASCGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDRAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKKNSKRWNPEIQYTSNYYKSNNVEFAVNTEGVYSEPRPIGTRYLTRNL

SEQ ID NO: 12 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPHDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFNVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSMEIEWELQKENSKRWNPEIQYTSNYYKSNNVEFAVNTEGVYSEPRPIGTRYLTRNL

SEQ ID NO: 13 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - DNA, 145 bp, synthetic construct
  misc_feature 1..145: note = Synthetic polynucleotide
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 15 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWERQKENSKRWNPEIQYTSNYYKSNNVEFAVNTEGVYSEPRPIGTRYLTRNL

SEQ ID NO: 16 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFADSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 736 aa, synthetic construct
  REGION 1..736: note = AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEIAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYCKSNNVEFAVNTEGVYSEPRPIGTRYLTRNL

SEQ ID NO: 18 - DNA, 145 bp, synthetic construct
  misc_feature 1..145: note = Synthetic polynucleotide
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 19 - DNA, 106 bp, synthetic construct
  misc_feature 1..106: note = Synthetic polynucleotide
  source 1..106: mol_type = other DNA
ctgcgcgctcgctcgctcactgaggccgcccgggcaaagcccgggcgtcgggcgacctttggtcgcccggcctcagtgagcgagcgagcgcgcagagagggagtgg

SEQ ID NO: 20 - DNA, 167 bp, synthetic construct
  misc_feature 1..167: note = Synthetic polynucleotide
  source 1..167: mol_type = other DNA
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 21 - DNA, 167 bp, synthetic construct
  misc_feature 1..167: note = Synthetic polynucleotide
  source 1..167: mol_type = other DNA
tacaaaacctccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacaggggggagag

SEQ ID NO: 22 - protein, 621 aa, synthetic construct
  REGION 1..621: note = AAV2 Rep
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 23 - DNA, 960 bp, synthetic construct
  misc_feature 1..960: note = Synthetic polynucleotide
  source 1..960: mol_type = other DNA
gcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaa

SEQ ID NO: 24 - DNA, 910 bp, synthetic construct
  misc_feature 1..910: note = Synthetic polynucleotide
  source 1..910: mol_type = other DNA
ctgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatata

SEQ ID NO: 25 - DNA, 192 bp, synthetic construct
  misc_feature 1..192: note = Synthetic polynucleotide
  source 1..192: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 26 - DNA, 255 bp, synthetic construct
  misc_feature 1..255: note = Synthetic polynucleotide
  source 1..255: mol_type = other DNA
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 27 - DNA, 448 bp, synthetic construct
  misc_feature 1..448: note = Synthetic polynucleotide
  source 1..448: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 28 - DNA, 1356 bp, synthetic construct
  misc_feature 1..1356: note = Synthetic polynucleotide
  source 1..1356: mol_type = other DNA
atgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagtgtgcgttacgacccttatacccagaggatcgaggtgctggataatacacagcagctgaagatcctggctgactcaatcaatagcgaaatcggaatcctgtgctccgccctgcagaaaatcaaa

SEQ ID NO: 29 - DNA, 93 bp, synthetic construct
  misc_feature 1..93: note = Synthetic polynucleotide
  source 1..93: mol_type = other DNA
ctctaaggtaaatataaaatttttaagtgtataatgtgttaaactactgattctaattgtttctctcttttagattccaacctttggaactga

SEQ ID NO: 30 - DNA, 26 bp, synthetic construct
  misc_feature 1..26: note = Synthetic polynucleotide
  source 1..26: mol_type = other DNA
ctgacctcttctcttcctcccacagg

SEQ ID NO: 31 - DNA, 198 bp, synthetic construct
  misc_feature 1..198: note = Synthetic polynucleotide
  source 1..198: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 32 - DNA, 143 bp, synthetic construct
  misc_feature 1..143: note = Synthetic polynucleotide
  source 1..143: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 33 - protein, 452 aa, Homo sapiens
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 34 - DNA, 122 bp, synthetic construct
  misc_feature 1..122: note = Synthetic polynucleotide
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 35 - DNA, 133 bp, synthetic construct
  misc_feature 1..133: note = Synthetic polynucleotide
  source 1..133: mol_type = other DNA
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 36 - DNA, 1676 bp, synthetic construct
  misc_feature 1..1676: note = Synthetic polynucleotide
  source 1..1676: mol_type = other DNA
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 37 - DNA, 960 bp, synthetic construct
  misc_feature 1..960: note = Synthetic polynucleotide
  source 1..960: mol_type = other DNA
gcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatccgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaa

SEQ ID NO: 38 - DNA, 911 bp, synthetic construct
  misc_feature 1..911: note = Synthetic polynucleotide
  source 1..911: mol_type = other DNA
ctgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatata

SEQ ID NO: 39 - DNA, 1168 bp, synthetic construct
  misc_feature 1..1168: note = Synthetic polynucleotide
  source 1..1168: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 40 - DNA, 72 bp, synthetic construct
  misc_feature 1..72: note = Synthetic polynucleotide
  source 1..72: mol_type = other DNA
agatctggcagcggagagggcagaggaagtcttctaacatgcggtgacgtggaggagaatcccggccctagg

SEQ ID NO: 41 - DNA, 95 bp, synthetic construct
  misc_feature 1..95: note = Synthetic polynucleotide
  source 1..95: mol_type = other DNA
cctctgctaaccatgttcatgccttcttctttttcctacagctcctgggcaacgtgctggttattgtgctgtctcatcattttggcaaagaattc

SEQ ID NO: 42 - DNA, 1873 bp, synthetic construct
  misc_feature 1..1873: note = Synthetic polynucleotide
  source 1..1873: mol_type = other DNA
gatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 43 - DNA, 4046 bp, synthetic construct
  misc_feature 1..4046: note = Synthetic polynucleotide
  source 1..4046: mol_type = other DNA
gcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaaccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagtgtgcgttacgacccttatacccagaggatcgaggtgctggataatacacagcagctgaagatcctggctgactcaatcaatagcgaaatcggaatcctgtgctccgccctgcagaaaatcaaatgagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagctttacgtacgatcgtcgactgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatata

SEQ ID NO: 44 - DNA, 913 bp, synthetic construct
  misc_feature 1..913: note = Synthetic polynucleotide
  source 1..913: mol_type = other DNA
ggcatcctaaaaaatattcagtggaaacgtaaaaacattaaagactgattaaacatcgcagcatgacacagatttagcaactgagcataaataatttgactcggatactgctccaaaatccgaagaggaccaatttcttccaggaggacaactacctcgtcctctgcagacccctctcctcggcagctgaaggagtgtggccaatctgcctccacctccccgcggaccccctactctcaggacctcctgcagcaccccaaactggaagtggccgctgcagacccaaggacgaggggcacgcgggagccggcagccctagtggagcggttggagatgttgaggtgggagggtcacccaggtggggtgaggctggggtaggtagcggagtgaacggcttccgaagctctgggccgcccccaggttggactaagcaggcgctctgtcttcgcccccgcccagggtgggcgtctcctgaggactccccgccacacctgacccgagaccgcgcgcccagcctagaacgcttccccgacccagcgtagggccgccgcgactggcgggcgagggtcggcgggaggcctggcgaacccgggggcgggaccaggcgggcaaggcccggctgccgcagcgccgctctgcgcgaggcggctccgccgcggcggagggatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcacc

SEQ ID NO: 45 - DNA, 4452 bp, synthetic construct
  misc_feature 1..4452: note = Synthetic polynucleotide
  source 1..4452: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaaccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagtgtgcgttacgacccttatacccagaggatcgaggtgctggataatacacagcagctgaagatcctggctgactcaatcaatagcgaaatcggaatcctgtgctccgccctgcagaaaatcaaatgagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagctttacgtacgatcgtcgactgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatatacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 46 - DNA, 380 bp, synthetic construct
  misc_feature 1..380: note = Synthetic polynucleotide
  source 1..380: mol_type = other DNA
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 47 - DNA, 1246 bp, synthetic construct
  misc_feature 1..1246: note = Synthetic polynucleotide
  source 1..1246: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcgg

SEQ ID NO: 48 - DNA, 1061 bp, synthetic construct
  misc_feature 1..1061: note = Synthetic polynucleotide
  source 1..1061: mol_type = other DNA
tagggaggtcctgcacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 49 - DNA, 953 bp, synthetic construct
  misc_feature 1..953: note = Synthetic polynucleotide
  source 1..953: mol_type = other DNA
aattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaag

SEQ ID NO: 50 - DNA, 541 bp, synthetic construct
  misc_feature 1..541: note = Synthetic polynucleotide
  source 1..541: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactga

SEQ ID NO: 51 - DNA, 1908 bp, synthetic construct
  misc_feature 1..1908: note = Synthetic polynucleotide
  source 1..1908: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagtgtgcgttacgacccttatacccagaggatcgaggtgctggataatacacagcagctgaagatcctggctgactcaatcaatagcgaaatcggaatcctgtgctccgccctgcagaaaatcaaatga

SEQ ID NO: 52 - DNA, 2156 bp, synthetic construct
  misc_feature 1..2156: note = Synthetic polynucleotide
  source 1..2156: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagtgtgcgttacgacccttatacccagaggatcgaggtgctggataatacacagcagctgaagatcctggctgactcaatcaatagcgaaatcggaatcctgtgctccgccctgcagaaaatcaaatgagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 53 - DNA, 1356 bp, Homo sapiens
  source 1..1356: mol_type = other DNA
atgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcaattcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctgtattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaag

SEQ ID NO: 54 - DNA, 173 bp, synthetic construct
  misc_feature 1..173: note = Synthetic polynucleotide
  source 1..173: mol_type = other DNA
actcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgt

SEQ ID NO: 55 - DNA, 100 bp, synthetic construct
  misc_feature 1..100: note = Synthetic polynucleotide
  source 1..100: mol_type = other DNA
ccaaaatcaacgggactttccaaaatgtcgtaataaccccgccccgttgacgcaaatgggcggtaggcgtgtacggtgggaggtctatataagcagagct

SEQ ID NO: 56 - DNA, 380 bp, synthetic construct; identical to SEQ ID NO: 46
  misc_feature 1..380: note = Synthetic polynucleotide
  source 1..380: mol_type = other DNA

SEQ ID NO: 57 - DNA, 1246 bp, synthetic construct; identical to SEQ ID NO: 47
  misc_feature 1..1246: note = Synthetic polynucleotide
  source 1..1246: mol_type = other DNA

SEQ ID NO: 58 - DNA, 201 bp, synthetic construct
  misc_feature 1..201: note = Synthetic polynucleotide
  source 1..201: mol_type = other DNA
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtgatcatcacttgtga

SEQ ID NO: 59 - DNA, 953 bp, synthetic construct; identical to SEQ ID NO: 49
  misc_feature 1..953: note = Synthetic polynucleotide
  source 1..953: mol_type = other DNA

SEQ ID NO: 60 - DNA, 1766 bp, synthetic construct
  misc_feature 1..1766: note = Synthetic polynucleotide
  source 1..1766: mol_type = other DNA
gtaaattttatggaatgtgaatcataattcaatttttcaacatgcgttaggagggacatttcaaactcttttttaccctagactttcctaccatcacccagagtatccagccaggaggggaggggctagagacaccagaagtttagcagggaggagggcgtagggattcggggaatgaagggatgggattcagactagggccaggacccagggatggagagaaagagatgagagtggtttgggggcttggtgacttagagaacagagctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcttaagcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 61 - DNA, 82 bp, synthetic construct
  misc_feature 1..82: note = Synthetic polynucleotide
  source 1..82: mol_type = other DNA
gtaagggtttaagggatggttggttggtggggtattaatgtttaattacctggagcacctgcctgaaatcactttttttcag

SEQ ID NO: 62 - DNA, 95 bp, synthetic construct; identical to SEQ ID NO: 41
  misc_feature 1..95: note = Synthetic polynucleotide
  source 1..95: mol_type = other DNA

SEQ ID NO: 63 - DNA, 1356 bp, synthetic construct
  misc_feature 1..1356: note = Synthetic polynucleotide
  source 1..1356: mol_type = other DNA
atgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaa

SEQ ID NO: 64 - DNA, 1873 bp, synthetic construct; identical to SEQ ID NO: 42
  misc_feature 1..1873: note = Synthetic polynucleotide
  source 1..1873: mol_type = other DNA

SEQ ID NO: 65 - DNA, 1061 bp, synthetic construct; identical to SEQ ID NO: 48
  misc_feature 1..1061: note = Synthetic polynucleotide
  source 1..1061: mol_type = other DNA

SEQ ID NO: 66 - DNA, 170 bp, synthetic construct
  misc_feature 1..170: note = Synthetic polynucleotide
  source 1..170: mol_type = other DNA
cgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatca

SEQ ID NO: 67 - DNA, 254 bp, synthetic construct
  misc_feature 1..254: note = Synthetic polynucleotide
  source 1..254: mol_type = other DNA
gggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatca

SEQ ID NO: 68 - DNA, 592 bp, synthetic construct
  misc_feature 1..592: note = Synthetic polynucleotide
  source 1..592: mol_type = other DNA
gtaaattttatggaatgtgaatcataattcaatttttcaacatgcgttaggagggacatttcaaactcttttttaccctagactttcctaccatcacccagagtatccagccaggaggggaggggctagagacaccagaagtttagcagggaggagggcgtagggattcggggaatgaagggatgggattcagactagggccaggacccagggatggagagaaagagatgagagtggtttgggggcttggtgacttagagaacagagctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 69 - DNA, 205 bp, synthetic construct
  misc_feature 1..205: note = Synthetic polynucleotide
  source 1..205: mol_type = other DNA
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagg

SEQ ID NO: 70 - DNA, 423 bp, synthetic construct
  misc_feature 1..423: note = Synthetic polynucleotide
  source 1..423: mol_type = other DNA
gctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggac

SEQ ID NO: 71 - DNA, 72 bp, synthetic construct
  misc_feature 1..72: note = Synthetic polynucleotide
  source 1..72: mol_type = other DNA
gggggaggctgctggtgaatattaaccaaggtcaccccagttatcggaggagcaaacaggggctaagtccac

SEQ ID NO: 72 - DNA, 913 bp, synthetic construct
  misc_feature 1..913: note = Synthetic polynucleotide
  source 1..913: mol_type = other DNA
tagggaggtcctgcacagaaggggaggagggggcagcagctgtctgaccactgttggtcttgcaacttgtgtccccaggttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccacccccaggattgtaactgaaatgcttcactggtgctccttttgttttaaggcattggatcttcatagctactgatcgtgcccaagcacacagtatctgcagcaaccacttaggcctccaggaatgtggtgaccattgaccctaattcattccccttcatggatcctatgtaaccatcctccaaaaagagctttcgcaaactcaaataaacacaggaaaggaagaccttcttatctttgagagtatatgtttagccctatagctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggac

SEQ ID NO: 73 - DNA, 37 bp, synthetic construct
  misc_feature 1..37: note = Synthetic polynucleotide
  source 1..37: mol_type = other DNA
gtagataagtagcatggcgggttaatcattaactaca

SEQ ID NO: 74 - DNA, 46 bp, synthetic construct
  misc_feature 1..46: note = Synthetic polynucleotide
  source 1..46: mol_type = other DNA
ggaggggtggagtcgtgacgtgaattacgtcatagggttagggagg

SEQ ID NO: 75 - DNA, 182 bp, synthetic construct
  misc_feature 1..182: note = Synthetic polynucleotide
  source 1..182: mol_type = other DNA
gtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 76 - DNA, 191 bp, synthetic construct
  misc_feature 1..191: note = Synthetic polynucleotide
  source 1..191: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggagg

SEQ ID NO: 77 - DNA, 3230 bp, synthetic construct
  misc_feature 1..3230: note = Synthetic polynucleotide
  source 1..3230: mol_type = other DNA
agcattagcttccatttatgcagtgtaaatggtgagaacagccccgactgaatacccagagcatcatctcgtctgtgtcattcatgcacataacatatctcagcgaggtggcccttctgtcctctttgcagagacccagccaccatactagtacctagagaactggctggatttcagccccgatacctccgggcttttgctcatgttcgcctcatagggtcatctgggtggttgcctaaggaaaagtatgtcatggagactaacttgcttggcattgaataaaaggtgagttgagagtggagcgtgtttaaattgcaatcctgcctctatttctgtgcttgcagggaacagtcatccttaattgctatcctccatcatcatcatgattatttctggtttttctctggttgcggagaatccatactccaggtattccaatgtctcagcattgccaggcctgtctgagcgtcaggatgtaggtagtctgggctctctgccttctattcttgtccaggatactctgccaaaagaatcatgttgtggctgccacccctcccacaaagcctcccgcttgggtcagtccaggactggagttgggtatggactgttcatgtctatccactgctacgtcagggcaacacccactgagagtgaccttgtagactgcagtgggagacacccttcaaaacctctcctctcctgtcctgagagccaggttaaaaccatcagccccgcatcctgagtgcaaacttttcctaaccctgctgctaagctagacacctcacttactgagagccagcatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagctttacgtacgatcgtcgagcagctgttgtcctggagaacggagtcctgagcagaaaactctcagactttgggcaggtaagcctgttgggcttccactgctaggagagaattggttccccacatgtgaaagcagtctgggaaatgctggtatttccagtctcctaaggctactaagaaatatgactttatttagaggcgaggaaaatgcccaggaagtcaactgatgagactagtcttaacaagttgaggatacagaaagttggggatctgagctgctaccaacatctgtgtgtctttgggtggctcattggtatcctctgcctattggctttatcttctgtacactgaaaggaaatggctggtccttagtcacctggggtgggagtccctatctctccagggatacttattcaatcctttcttctgggtatcaaaatgacaagcttgtaagaaactgtcctctttcggctttcaggaggtgatgtcgcatgaagagaatttggggggggggacttactcagaaccaaggagggagaaattaaacagagagggaaatgaacaggagttagcccggagcctgaagcaccttggggattatgctgggggtggagggaatccattgtcctccctagggagggcttgcagaacatgttcttttctgtgatatttgtactttccccagattgcaaatcatggtttgtacactgagattcagtctctggaggtaatatgccttttctagcttttccttggacaggactaaggggttgagggttgcctggagtcagagaaattaactgtgttaaagaaggttgatatga

SEQ ID NO: 78 - DNA, 3637 bp, synthetic construct
  misc_feature 1..3637: note = Synthetic polynucleotide
  source 1..3637: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcagcattagcttccatttatgcagtgtaaatggtgagaacagccccgactgaatacccagagcatcatctcgtctgtgtcattcatgcacataacatatctcagcgaggtggcccttctgtcctctttgcagagacccagccaccatactagtacctagagaactggctggatttcagccccgatacctccgggcttttgctcatgttcgcctcatagggtcatctgggtggttgcctaaggaaaagtatgtcatggagactaacttgcttggcattgaataaaaggtgagttgagagtggagcgtgtttaaattgcaatcctgcctctatttctgtgcttgcagggaacagtcatccttaattgctatcctccatcatcatcatgattatttctggtttttctctggttgcggagaatccatactccaggtattccaatgtctcagcattgccaggcctgtctgagcgtcaggatgtaggtagtctgggctctctgccttctattcttgtccaggatactctgccaaaagaatcatgttgtggctgccacccctcccacaaagcctcccgcttgggtcagtccaggactggagttgggtatggactgttcatgtctatccactgctacgtcagggcaacacccactgagagtgaccttgtagactgcagtgggagacacccttcaaaacctctcctctcctgtcctgagagccaggttaaaaccatcagccccgcatcctgagtgcaaacttttcctaaccctgctgctaagctagacacctcacttactgagagccagcatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagctttacgtacgatcgtcgagcagctgttgtcctggagaacggagtcctgagcagaaaactctcagactttgggcaggtaagcctgttgggcttccactgctaggagagaattggttccccacatgtgaaagcagtctgggaaatgctggtatttccagtctcctaaggctactaagaaatatgactttatttagaggcgaggaaaatgcccaggaagtcaactgatgagactagtcttaacaagttgaggatacagaaagttggggatctgagctgctaccaacatctgtgtgtctttgggtggctcattggtatcctctgcctattggctttatcttctgtacactgaaaggaaatggctggtccttagtcacctggggtgggagtccctatctctccagggatacttattcaatcctttcttctgggtatcaaaatgacaagcttgtaagaaactgtcctctttcggctttcaggaggtgatgtcgcatgaagagaatttggggggggggacttactcagaaccaaggagggagaaattaaacagagagggaaatgaacaggagttagcccggagcctgaagcaccttggggattatgctgggggtggagggaatccattgtcctccctagggagggcttgcagaacatgttcttttctgtgatatttgtactttccccagattgcaaatcatggtttgtacactgagattcagtctctggaggtaatatgccttttctagcttttccttggacaggactaaggggttgagggttgcctggagtcagagaaatttgtgttaaagaaggttgatatgaaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 79 - DNA, 3776 bp, synthetic construct
  misc_feature 1..3776: note = Synthetic polynucleotide
  source 1..3776: mol_type = other DNA
agcattagcttccatttatgcagtgtaaatggtgagaacagccccgactgaatacccagagcatcatctcgtctgtgtcattcatgcacataacatatctcagcgaggtggcccttctgtcctctttgcagagacccagccaccatactagtacctagagaactggctggatttcagccccgatacctccgggcttttgctcatgttcgcctcatagggtcatctgggtggttgcctaaggaaaagtatgtcatggagactaacttgcttggcattgaataaaaggtgagttgagagtggagcgtgtttaaattgcaatcctgcctctatttctgtgcttgcagggaacagtcatccttaattgctatcctccatcatcatcatgattatttctggtttttctctggttgcggagaatccatactccaggtattccaatgtctcagcattgccaggcctgtctgagcgtcaggatgtaggtagtctgggctctctgccttctattcttgtccaggatactctgccaaaagaatcatgttgtggctgccacccctcccacaaagcctcccgcttgggtcagtccaggactggagttgggtatggactgttcatgtctatccactgctacgtcagggcaacacccactgagagtgaccttgtagactgcagtgggagacacccttcaaaacctctcctctcctgtcctgagagccaggttaaaaccatcagccccgcatcctgagtgcaaacttttcctaaccctgctgctaagctagacaccccacttactgagagccagcccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagtgtgcgttacgacccttatacccagaggatcgaggtgctggataatacacagcagctgaagatcctggctgactcaatcaatagcgaaatcggaatcctgtgctccgccctgcagaaaatcaaatgagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagctttacgtacgatcgtcgagcagctgttgtcctggagaacggagtcctgagcagaaaactctcagactttgggcaggtaagcctgttgggcttccactgctaggagagaattggttccccacatgtgaaagcagtctgggaaatgctggtatttccagtctcctaaggctactaagaaatatgactttatttagaggcgaggaaaatgcccaggaagtcaactgatgagactagtcttaacaagttgaggatacagaaagttggggatctgagctgctaccaacatctgtgtgtctttgggtggctcattggtatcctctgcctattggctttatcttctgtacactgaaaggaaatggctggtccttagtcacctggggtgggagtccctatctctccagggatacttattcaatcctttcttctgggtatcaaaatgacaagcttgtaagaaactgtcctctttcggctttcaggaggtgatgtcgcatgaagagaatttggggggggggacttactcagaaccaaggagggagaaattaaacagagagggaaatgaacaggagttagcccggagcctgaagcaccttggggattatgctgggggtggagggaatccattgtcctccctagggagggcttgcagaacatgttcttttctgtgatatttgtactttccccagattgcaaatcatggtttgtacactgagattcagtctctggaggtaatatgccttttctagcttttccttggacaggactaaggggttgagggttgcctggagtcagagaaatttgtgttaaagaaggttgatatga

SEQ ID NO: 80 - DNA, 4186 bp, synthetic construct
  misc_feature 1..4186: note = Synthetic polynucleotide
  source 1..4186: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcagcattagcttccatttatgcagtgtaaatggtgagaacagccccgactgaatacccagagcatcatctcgtctgtgtcattcatgcacataacatatctcagcgaggtggcccttctgtcctctttgcagagacccagccaccatactagtacctagagaactggctggatttcagccccgatacctccgggcttttgctcatgttcgcctcatagggtcatctgggtggttgcctaaggaaaagtatgtcatggagactaacttgcttggcattgaataaaaggtgagttgagagtggagcgtgtttaaattgcaatcctgcctctatttctgtgcttgcagggaacagtcatccttaattgctatcctccatcatcatcatgattatttctggtttttctctggttgcggagaatccatactccaggtattccaatgtctcagcattgccaggcctgtctgagcgtcaggatgtaggtagtctgggctctctgccttctattcttgtccaggatactctgccaaaagaatcatgttgtggctgccacccctcccacaaagcctcccgcttgggtcagtccaggactggagttgggtatggactgttcatgtctatccactgctacgtcagggcaacacccactgagagtgaccttgtagactgcagtgggagacacccttcaaaacctctcctctcctgtcctgagagccaggttaaaaccatcagccccgcatcctgagtgcaaacttttcctaaccctgctgctaagctagacaccccacttactgagagccagcccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagtgtgcgttacgacccttatacccagaggatcgaggtgctggataatacacagcagctgaagatcctggctgactcaatcaatagcgaaatcggaatcctgtgctccgccctgcagaaaatcaaatgagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagctttacgtacgatcgtcgagcagctgttgtcctggagaacggagtcctgagcagaaaactctcagactttgggcaggtaagcctgttgggcttccactgctaggagagaattggttccccacatgtgaaagcagtctgggaaatgctggtatttccagtctcctaaggctactaagaaatatgactttatttagaggcgaggaaaatgcccaggaagtcaactgatgagactagtcttaacaagttgaggatacagaaagttggggatctgagctgctaccaacatctgtgtgtctttgggtggctcattggtatcctctgcctattggctttatcttctgtacactgaaaggaaatggctggtccttagtcacctggggtgggagtccctatctctccagggatacttattcaatcctttcttctgggtatcaaaatgacaagcttgtaagaaactgtcctctttcggctttcaggaggtgatgtcgcatgaagagaatttggggggggggacttactcagaaccaaggagggagaaattaaacagagagggaaatgaacaggagttagcccggagcctgaagcaccttggggattatgctgggggtggagggaatccattgtcctccctagggagggcttgcagaacatgttcttttctgtgatatttgtactttccccagattgcaaatcatggtttgtacactgagattcagtctctggaggtaatatgccttttctagcttttccttggacaggactaaggggttgagggttgcctggagtcagagaaatttgtgttaaagaaggttgatatgaaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 81 - DNA, 3713 bp, synthetic construct
  misc_feature 1..3713: note = Synthetic polynucleotide
  source 1..3713: mol_type = other DNA
gcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaaaagcttctgacctcttctcttcctcccacagggcggtaccagatctggcagcggagagggcagaggaagtcttctaacatgcggtgacgtggaggagaatcccggccctaggggtacctccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaaggtaagcctatccctaaccctctcctcggtctcgattctacgtgatcttgtggaaaggacgaaacaccggggaattcaaggcctctcgagcctctagaatccccgagacgtttcgtctcgggatcactatagtgagtcgtattacgtacacagtgcaggggaaagaatagtagagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaactgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatata

SEQ ID NO: 82 - DNA, 4139 bp, synthetic construct
  misc_feature 1..4139: note = Synthetic polynucleotide
  source 1..4139: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaaaagcttctgacctcttctcttcctcccacagggcggtaccagatctggcagcggagagggcagaggaagtcttctaacatgcggtgacgtggaggagaatcccggccctaggggtacctccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaaggtaagcctatccctaaccctctcctcggtctcgattctacgtgatcttgtggaaaggacgaaacaccggggaattcaaggcctctcgagcctctagaatccccgagacgtttcgtctcgggatcactatagtgagtcgtattacgtacacagtgcaggggaaagaatagtagagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaactgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatatactagatacgtagataagtagcctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 83 - DNA, 1353 bp, synthetic construct
  misc_feature 1..1353: note = Synthetic polynucleotide
  source 1..1353: mol_type = other DNA
tccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaa

SEQ ID NO: 84 - DNA, 800 bp, synthetic construct
  misc_feature 1..800: note = Synthetic polynucleotide
  source 1..800: mol_type = other DNA
agcattagcttccatttatgcagtgtaaatggtgagaacagccccgactgaatacccagagcatcatctcgtctgtgtcattcatgcacataacatatctcagcgaggtggcccttctgtcctctttgcagagacccagccaccatactagtacctagagaactggctggatttcagccccgatacctccgggcttttgctcatgttcgcctcatagggtcatctgggtggttgcctaaggaaaagtatgtcatggagactaacttgcttggcattgaataaaaggtgagttgagagtggagcgtgtttaaattgcaatcctgcctctatttctgtgcttgcagggaacagtcatccttaattgctatcctccatcatcatcatgattatttctggtttttctctggttgcggagaatccatactccaggtattccaatgtctcagcattgccaggcctgtctgagcgtcaggatgtaggtagtctgggctctctgccttctattcttgtccaggatactctgccaaaagaatcatgttgtggctgccacccctcccacaaagcctcccgcttgggtcagtccaggactggagttgggtatggactgttcatgtctatccactgctacgtcagggcaacacccactgagagtgaccttgtagactgcagtgggagacacccttcaaaacctctcctctcctgtcctgagagccaggttaaaaccatcagccccgcatcctgagtgcaaacttttcctaaccctgctgctaagctagacacctcacttactgagagccagc

SEQ ID NO: 85 - DNA, 800 bp, synthetic construct
  misc_feature 1..800: note = Synthetic polynucleotide
  source 1..800: mol_type = other DNA
agcattagcttccatttatgcagtgtaaatggtgagaacagccccgactgaatacccagagcatcatctcgtctgtgtcattcatgcacataacatatctcagcgaggtggcccttctgtcctctttgcagagacccagccaccatactagtacctagagaactggctggatttcagccccgatacctccgggcttttgctcatgttcgcctcatagggtcatctgggtggttgcctaaggaaaagtatgtcatggagactaacttgcttggcattgaataaaaggtgagttgagagtggagcgtgtttaaattgcaatcctgcctctatttctgtgcttgcagggaacagtcatccttaattgctatcctccatcatcatcatgattatttctggtttttctctggttgcggagaatccatactccaggtattccaatgtctcagcattgccaggcctgtctgagcgtcaggatgtaggtagtctgggctctctgccttctattcttgtccaggatactctgccaaaagaatcatgttgtggctgccacccctcccacaaagcctcccgcttgggtcagtccaggactggagttgggtatggactgttcatgtctatccactgctacgtcagggcaacacccactgagagtgaccttgtagactgcagtgggagacacccttcaaaacctctcctctcctgtcctgagagccaggttaaaaccatcagccccgcatcctgagtgcaaacttttcctaaccctgctgctaagctagacaccccacttactgagagccagc

SEQ ID NO: 86 - DNA, 800 bp, synthetic construct
  misc_feature 1..800: note = Synthetic polynucleotide
  source 1..800: mol_type = other DNA
gcagctgttgtcctggagaacggagtcctgagcagaaaactctcagactttgggcaggtaagcctgttgggcttccactgctaggagagaattggttccccacatgtgaaagcagtctgggaaatgctggtatttccagtctcctaaggctactaagaaatatgactttatttagaggcgaggaaaatgcccaggaagtcaactgatgagactagtcttaacaagttgaggatacagaaagttggggatctgagctgctaccaacatctgtgtgtctttgggtggctcattggtatcctctgcctattggctttatcttctgtacactgaaaggaaatggctggtccttagtcacctggggtgggagtccctatctctccagggatacttattcaatcctttcttctgggtatcaaaatgacaagcttgtaagaaactgtcctctttcggctttcaggaggtgatgtcgcatgaagagaatttggggggggggacttactcagaaccaaggagggagaaattaaacagagagggaaatgaacaggagttagcccggagcctgaagcaccttggggattatgctgggggtggagggaatccattgtcctccctagggagggcttgcagaacatgttcttttctgtgatatttgtactttccccagattgcaaatcatggtttgtacactgagattcagtctctggaggtaatatgccttttctagcttttccttggacaggactaaggggttgagggttgcctggagtcagagaaatttgtgttaaagaaggttgatatga

(19) United States
(12) Patent Application Publication
(10) Pub. No.: US 2025/0090691 A1
(43) Pub. Date: Mar. 20, 2025

(54) ADENO-ASSOCIATED VIRUS COMPOSITIONS FOR RESTORING PAH GENE FUNCTION AND METHODS OF USE THEREOF

(71) Applicant: Homology Medicines, Inc., Bedford, MA (US)

(72) Inventors: Jason Boke Wright, Bedford, MA (US); Danielle Lauren Sookiasian, Bedford, MA (US); Thia Baboval St. Martin, Bedford, MA (US); Omar Francone, Bedford, MA (US); Albert Barnes Seymour, Bedford, MA (US)

(21) Appl. No.: 18/784,450

(22) Filed: Jul. 25, 2024

Related U.S. Application Data
(62) Division of application No. 17/303,343, filed on May 27, 2021, now Pat. No. 12,076,420.
(60) Provisional application No. 63/117,252, filed on Nov. 23, 2020.
(60) Provisional application No. 63/030,341, filed on May 27, 2020.

(51) Int. Cl. A61K 48/00; A61K 38/44; C12N 15/86

(52) U.S. Cl. CPC A61K 48/0066; A61K 38/44; C12N 15/86; C12N 2750/14143; C12N 2750/14151; C12N 2830/50; C12Y 114/16001

(57) ABSTRACT

Provided herein are recombinant adeno-associated virus (rAAV) compositions that can restore phenylalanine hydroxylase (PAH) gene function in cells, and methods for using the same to treat diseases associated with reduction of PAH gene function (e.g., PKU). Also provided are nucleic acids, vectors, packaging systems, and methods for making the adeno-associated virus compositions.

### RELATED APPLICATIONS

This application is a division of U.S. patent application Ser. No. 17/303,343, filed May 27, 2021, which claims priority to U.S. Provisional Patent Application Ser. Nos. 63/030,341, filed May 27, 2020, and 63/117,252, filed Nov. 23, 2020, the entire disclosures of which are hereby incorporated herein by reference.

### SEQUENCE LISTING

This application contains a sequence listing which has been submitted electronically in XML format and is hereby incorporated by reference in its entirety (said XML copy, created on Dec. 5, 2024, is named “211549_seqlist.xml” and is 190,643 bytes in size).

### BACKGROUND

Phenylketonuria (PKU) is an autosomal recessive genetic disorder where the majority of cases are caused by mutations in the phenylalanine hydroxylase (PAH) gene. The PAH gene encodes a hepatic enzyme that catalyzes the hydroxylation of L-phenylalanine (Phe) to L-tyrosine (Tyr) upon multimerization. Reduction or loss of PAH activity leads to phenylalanine accumulation and its conversion into phenylpyruvate (also known as phenylketone). This abnormality in phenylalanine metabolism impairs neuronal maturation and the synthesis of myelin, resulting in mental retardation, seizures, and other serious medical problems.

Currently, there is no cure for PKU. The standard of care is diet management by minimizing foods that contain high amounts of phenylalanine. Dietary management from birth with a low phenylalanine formula largely prevents the development of the neurological consequences of the disorder. However, even on a low-protein diet, children still suffer from growth retardation, and adults often have osteoporosis and vitamin deficiencies. Moreover, adherence to life-long dietary treatment is difficult, particularly beyond school age.

New treatment strategies have recently emerged, including large neutral amino acid (LNAA) supplementation, cofactor tetrahydrobiopterin therapy, enzyme replacement therapy, and genetically modified probiotic therapy. However, these strategies suffer from shortcomings. The LNAA supplementation is suitable only for adults not adhering to a low Phe diet. The cofactor tetrahydrobiopterin can only be used in some mild forms of PKU. Enzyme replacement by administration of a substitute for PAH, e.g., phenylalanine ammonia-lyase (PAL), can lead to immune responses that reduce the efficacy and/or cause side effects. As to genetically modified probiotic therapy, the pathogenicity of PAL-expressing E. coli has been a concern.

Gene therapy provides a unique opportunity to cure PKU. Retroviral vectors, including lentiviral vectors, are capable of integrating nucleic acids into host cell genomes. However, these vectors may raise safety concerns due to their non-targeted insertion into the genome. For example, there is a risk of the vector disrupting a tumor suppressor gene or activating an oncogene, thereby causing a malignancy. Indeed, in a clinical trial for treating X-linked severe combined immunodeficiency (SCID) by transducing CD34⁺ bone marrow precursors with a gammaretroviral vector, four out of ten patients developed leukemia (Hacein-Bey-Abina et al. J Clin Invest. (2008) 118 (9): 3132-42).

It has also been speculated that nuclease-based gene editing technologies, such as meganucleases, zinc finger nucleases (ZFNs), transcription activator-like effector nucleases (TALENs), and clustered, regularly interspaced, short palindromic repeat (CRISPR) technology, may be used to correct defects in the PAH gene in PKU patients. However, each of these technologies raises safety concerns due to the potential for off-target mutation of sites in the human genome similar in sequence to the intended target site.

Accordingly, there is a need in the art for improved gene therapy compositions and methods that can efficiently and safely restore PAH gene function in PKU patients.

### SUMMARY

Provided herein are recombinant adeno-associated virus (rAAV) compositions that can restore PAH gene function in cells, and methods for using the same to treat diseases associated with reduction of PAH gene function (e.g., PKU). Also provided are nucleic acids, vectors, packaging systems, and methods for making the adeno-associated virus compositions. The rAAV compositions provided herein are particularly advantageous in that they can efficiently edit the genome of cells (e.g., liver cells) in a subject to express PAH under the control of a liver-specific promoter, and thereby offer a potential cure for PKU patients.

Accordingly, in one aspect, the instant disclosure provides a recombinant adeno-associated virus (rAAV) comprising:
(a)) an AAV capsid comprising an AAV capsid protein; and
(b) an rAAV genome comprising: (i) an editing element for editing a target locus in a PAH gene, comprising at least a portion of a PAH coding sequence operably linked to a transcriptional regulatory element; (ii) a 5′ homology arm nucleotide sequence position 5′ of the editing element, having homology to a first genomic region 5′ to the target locus; and (iii) a 3′ homology arm nucleotide sequence positioned 3′ of the editing element, having homology to a second genomic region 3′ to the target locus.

In certain embodiments, the editing element comprises a PAH coding sequence. In certain embodiments, the PAH coding sequence encodes an amino acid sequence set forth in SEQ ID NO: 33. In certain embodiments, the PAH coding sequence comprises the nucleotide sequence set forth in SEQ ID NO: 53. In certain embodiments, the PAH coding sequence is silently altered. In certain embodiments, the PAH coding sequence comprises a nucleotide sequence that is at least 85%, 86%, 87%, 88%, 89%, 90%, 91%, 92%, 93%, 94%, 95%, 96%, 97%, 98%, or 99% identical to the nucleotide sequence set forth in SEQ ID NO: 28, 63, or 83. In certain embodiments, the PAH coding sequence comprises the nucleotide sequence set forth in SEQ ID NO: 28. In certain embodiments, the PAH coding sequence comprises the nucleotide sequence set forth in SEQ ID NO: 63. In certain embodiments, the PAH coding sequence comprises the nucleotide sequence set forth in SEQ ID NO: 83.

In certain embodiments, the transcriptional regulatory element is capable of mediating transcription in a hepatocyte, a renal cell, or a cell in the brain, pituitary gland, adrenal gland, pancreas, urinary bladder, gallbladder, colon, small intestine, or breast. In certain embodiments, the transcriptional regulatory element is endogenous to the PAH gene. In certain embodiments, the transcriptional regulatory element is exogenous to the PAH gene. In certain embodiments, the transcriptional regulatory element is liver specific, optionally wherein the transcriptional regulatory element comprises one or more elements selected from the group consisting of a human albumin promoter, a human transthyretin (TTR) promoter, a human ApoE/C-I hepatic control region (HCR) 1 or 2, a human ApoH promoter, a human SERPINA1 (hAAT) promoter, and a hepatic specific regulatory module thereof. In certain embodiments, the transcriptional regulatory element comprises a nucleotide sequence at least 90% identical to a sequence selected from the group consisting of SEQ ID NO: 25, 26, 27, or 69. In certain embodiments, the transcriptional regulatory element comprises a nucleotide sequence at least 90% identical to the nucleotide sequence set forth in SEQ ID NO: 27. In certain embodiments, the transcriptional regulatory element comprises the nucleotide sequence set forth in SEQ ID NO: 27. In certain embodiments, the transcriptional regulatory element consists of the nucleotide sequence set forth in SEQ ID NO: 27.

In certain embodiments, the editing element further comprises an intron element positioned 5′ to the PAH coding sequence and 3′ to the transcriptional regulatory element. In certain embodiments, the intron element is an exogenous intron element, optionally wherein the exogenous intron element is an SV40 intron element. In certain embodiments, the SV40 intron element comprises a nucleotide sequence at least 90% identical to the nucleotide sequence set forth in SEQ ID NO: 29. In certain embodiments, the SV40 intron element comprises the nucleotide sequence set forth in SEQ ID NO: 29. In certain embodiments, the SV40 intron element consists of the nucleotide sequence set forth in SEQ ID NO: 29.

In certain embodiments, the editing element further comprises a polyadenylation sequence 3′ to the PAH coding sequence. In certain embodiments, the polyadenylation sequence is an exogenous polyadenylation sequence, optionally wherein the exogenous polyadenylation sequence is an SV40 polyadenylation sequence. In certain embodiments, the SV40 polyadenylation sequence comprises a nucleotide sequence at least 90% identical to the nucleotide sequence set forth in SEQ ID NO: 31. In certain embodiments, the SV40 polyadenylation sequence comprises the nucleotide sequence set forth in SEQ ID NO: 31. In certain embodiments, the SV40 polyadenylation sequence consists of the nucleotide sequence set forth in SEQ ID NO: 31.

In certain embodiments, the nucleotide 5′ to the target locus is in an intron of a PAH gene. In certain embodiments, the nucleotide 5′ to the target locus is in intron 1 of a PAH gene. In certain embodiments, the nucleotide 3′ to the target locus is in an intron of a PAH gene. In certain embodiments, the nucleotide 3′ to the target locus is in intron 1 of a PAH gene.

In certain embodiments, the PAH gene is a human PAH gene. In certain embodiments, the human PAH gene is wild-type. In certain embodiments, the human PAH gene is a variant PAH gene.

In certain embodiments, the editing element comprises a nucleotide sequence selected from the group consisting of SEQ ID NO: 25, 26, 27, 28, 29, 31, 50, 51, 52, 69, or 70.

In certain embodiments, the 5′ homology arm nucleotide sequence is at least 90%, 91%, 92%, 93%, 94%, 95%, 96%, 97%, 98%, 99%, or 100% identical to the first genomic region. In certain embodiments, the 3′ homology arm nucleotide sequence is at least 90%, 91%, 92%, 93%, 94%, 95%, 96%, 97%, 98%, 99%, or 100% identical to the second genomic region. In certain embodiments, the first genomic region is located in a first editing window, and the second genomic region is located in a second editing window. In certain embodiments, the first editing window consists of the region of the human genome corresponding to the nucleotide sequence set forth in SEQ ID NO: 37. In certain embodiments, the second editing window consists of the region of the human genome corresponding to the nucleotide sequence set forth in SEQ ID NO: 38. In certain embodiments, the first genomic region consists of the region of the human genome corresponding to the nucleotide sequence set forth in SEQ ID NO: 37. In certain embodiments, the second genomic region consists of the region of the human genome corresponding to the nucleotide sequence set forth in SEQ ID NO: 38. In certain embodiments, each of the 5′ and 3′ homology arm nucleotide sequences independently has a length of about 100 to about 2000 nucleotides. In certain embodiments, the 5′ homology arm comprises a nucleotide sequence that is at least 90%, 91%, 92%, 93%, 94%, 95%, 96%, 97%, 98%, 99%, or 100% identical to the nucleotide sequence set forth in SEQ ID NO: 23. In certain embodiments, the 5′ homology arm comprises the nucleotide sequence set forth in SEQ ID NO: 23. In certain embodiments, the nucleotide sequence of the 5′ homology arm consists of the nucleotide sequence set forth in SEQ ID NO: 23. In certain embodiments, the 3′ homology arm comprises a nucleotide sequence that is at least 90%, 91%, 92%, 93%, 94%, 95%, 96%, 97%, 98%, 99%, or 100% identical to the nucleotide sequence set forth in SEQ ID NO: 24. In certain embodiments, the 3′ homology arm comprises the nucleotide sequence set forth in SEQ ID NO: 24. In certain embodiments, the nucleotide sequence of the 3′ homology arm consists of the nucleotide sequence set forth in SEQ ID NO: 24.

In certain embodiments, the rAAV genome comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 43. In certain embodiments, the rAAV genome comprises the nucleotide sequence set forth in SEQ ID NO: 43.

In certain embodiments, the rAAV genome further comprises a 5′ inverted terminal repeat (5′ ITR) nucleotide sequence 5′ of the 5′ homology arm nucleotide sequence, and a 3′ inverted terminal repeat (3′ ITR) nucleotide sequence 3′ of the 3′ homology arm nucleotide sequence. In certain embodiments, the 5′ ITR nucleotide sequence is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 14, and the 3′ ITR nucleotide sequence is at least 85%, 86%, 87%, 88%, 89%, 90%, 91%, 92%, 93%, 94%, 95%, 96%, 97%, 98%, 99% or 100% identical to the nucleotide sequence set forth in SEQ ID NO: 18.

In certain embodiments, the rAAV genome comprises the nucleotide sequence set forth in SEQ ID NO: 73 and/or 74. In certain embodiments, the rAAV genome comprises the nucleotide sequence set forth in SEQ ID NO: 75 and/or 76.

In certain embodiments, the rAAV genome comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 45. In certain embodiments, the rAAV genome comprises the nucleotide sequence set forth in SEQ ID NO: 45. In certain embodiments, the nucleotide sequence of the rAAV genome consists of the nucleotide sequence set forth in SEQ ID NO: 45.

In certain embodiments, the AAV capsid protein comprises an amino acid sequence that is at least 95% identical to the amino acid sequence of amino acids 203-736 of SEQ ID NO: 1, 2, 3, 4, 5, 6, 7, 8, 9, 10, 11, 12, 13, 15, 16, or 17. In certain embodiments, the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments:
(a) the amino acid in the capsid protein corresponding to amino acid 626 of SEQ ID NO: 16 is G, and the amino acid in the capsid protein corresponding to amino acid 718 of SEQ ID NO: 16 is G;
(b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
(c) the amino acid in the capsid protein corresponding to amino acid 505 of SEQ ID NO: 16 is R, and the amino acid in the capsid protein corresponding to amino acid 687 of SEQ ID NO: 16 is R;
(d) the amino acid in the capsid protein corresponding to amino acid 346 of SEQ ID NO: 16 is A, and the amino acid in the capsid protein corresponding to amino acid 505 of SEQ ID NO: 16 is R; or
(e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the capsid protein comprises the amino acid sequence of amino acids 203-736 of SEQ ID NO: 1, 2, 3, 4, 5, 6, 7, 8, 9, 10, 11, 12, 13, 15, 16, or 17.

In certain embodiments, the AAV capsid protein comprises an amino acid sequence that is at least 95% identical to the amino acid sequence of amino acids 138-736 of SEQ ID NO: 1, 2, 3, 4, 5, 6, 7, 8, 9, 10, 11, 12, 13, 15, 16, or 17. In certain embodiments, the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments,
(a) the amino acid in the capsid protein corresponding to amino acid 626 of SEQ ID NO: 16 is G, and the amino acid in the capsid protein corresponding to amino acid 718 of SEQ ID NO: 16 is G;
(b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
(c) the amino acid in the capsid protein corresponding to amino acid 505 of SEQ ID NO: 16 is R, and the amino acid in the capsid protein corresponding to amino acid 687 of SEQ ID NO: 16 is R;
(d) the amino acid in the capsid protein corresponding to amino acid 346 of SEQ ID NO: 16 is A, and the amino acid in the capsid protein corresponding to amino acid 505 of SEQ ID NO: 16 is R; or
(e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the capsid protein comprises the amino acid sequence of amino acids 138-736 of SEQ ID NO: 2, 3, 4, 5, 6, 7, 9, 10, 11, 12, 13, 15, 16, or 17.

In certain embodiments, the AAV capsid protein comprises an amino acid sequence that is at least 95% identical to the amino acid sequence of amino acids 1-736 of SEQ ID NO: 1, 2, 3, 4, 5, 6, 7, 8, 9, 10, 11, 12, 13, 15, 16, or 17. In certain embodiments, the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments:
(a) the amino acid in the capsid protein corresponding to amino acid 2 of SEQ ID NO: 16 is T, and the amino acid in the capsid protein corresponding to amino acid 312 of SEQ ID NO: 16 is Q;
(b) the amino acid in the capsid protein corresponding to amino acid 65 of SEQ ID NO: 16 is I, and the amino acid in the capsid protein corresponding to amino acid 626 of SEQ ID NO: 16 is Y;
(c) the amino acid in the capsid protein corresponding to amino acid 77 of SEQ ID NO: 16 is R, and the amino acid in the capsid protein corresponding to amino acid 690 of SEQ ID NO: 16 is K;
(d) the amino acid in the capsid protein corresponding to amino acid 119 of SEQ ID NO: 16 is L, and the amino acid in the capsid protein corresponding to amino acid 468 of SEQ ID NO: 16 is S;
(e) the amino acid in the capsid protein corresponding to amino acid 626 of SEQ ID NO: 16 is G, and the amino acid in the capsid protein corresponding to amino acid 718 of SEQ ID NO: 16 is G;
(f) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
(g) the amino acid in the capsid protein corresponding to amino acid 505 of SEQ ID NO: 16 is R, and the amino acid in the capsid protein corresponding to amino acid 687 of SEQ ID NO: 16 is R;
(h) the amino acid in the capsid protein corresponding to amino acid 346 of SEQ ID NO: 16 is A, and the amino acid in the capsid protein corresponding to amino acid 505 of SEQ ID NO: 16 is R; or
(i) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the capsid protein comprises the amino acid sequence of amino acids 1-736 of SEQ ID NO: 1, 2, 3, 4, 5, 6, 7, 8, 9, 10, 11, 12, 13, 15, 16, or 17.

In certain embodiments, the integration efficiency of the editing element into the target locus is at least 1% when the AAV is administered to a mouse implanted with human hepatocytes in the absence of an exogenous nuclease under standard AAV administration conditions. In certain embodiments, the allelic frequency of integration of the editing element into the target locus is at least 0.5% when the AAV is administered to a mouse implanted with human hepatocytes in the absence of an exogenous nuclease under standard AAV administration conditions.

In another aspect, the instant disclosure provides a pharmaceutical composition comprising an rAAV disclosed herein.

In another aspect, the instant disclosure provides a polynucleotide comprising the nucleic acid sequence set forth in SEQ ID NO: 43, 45, 51, or 52.

In another aspect, the instant disclosure provides a method for treating a subject having phenylketonuria (PKU), the method comprising administering to the subject an effective amount of an rAAV, or pharmaceutical composition disclosed herein. In certain embodiments, the rAAV or pharmaceutical composition is administered intravenously. In certain embodiments, the PKU is associated with a PAH gene mutation. In certain embodiments, the subject is a human subject.

In another aspect, the instant disclosure provides a packaging system for preparation of an rAAV, wherein the packaging system comprises: (a) a first nucleotide sequence encoding one or more AAV Rep proteins; (b) a second nucleotide sequence encoding a capsid protein disclosed herein; and (c) a third nucleotide sequence comprising an rAAV genome sequence of an rAAV disclosed herein. In certain embodiments, the packaging system comprises a first vector comprising the first nucleotide sequence and the second nucleotide sequence, and a second vector comprising the third nucleotide sequence. In certain embodiments, the packaging system further comprises a fourth nucleotide sequence comprising one or more helper virus genes. In certain embodiments, the fourth nucleotide sequence is comprised within a third vector. In certain embodiments, the fourth nucleotide sequence comprises one or more genes from a virus selected from the group consisting of adenovirus, herpes virus, vaccinia virus, and cytomegalovirus (CMV). In certain embodiments, the first vector, second vector, and/or the third vector is a plasmid.

In another aspect, the instant disclosure provides a method for recombinant preparation of an rAAV, the method comprising introducing a packaging system disclosed herein into a cell under conditions whereby the rAAV is produced.

An rAAV, pharmaceutical composition, or polynucleotide disclosed herein, for use as a medicament. An rAAV, pharmaceutical composition, or polynucleotide disclosed herein, for use in the treatment of PKU. An rAAV, pharmaceutical composition, or polynucleotide disclosed herein, for use in a method of treating a subject having PKU, the method comprising administering to the subject an effective amount of the rAAV, the pharmaceutical composition, or the polynucleotide.

### BRIEF DESCRIPTION OF THE DRAWINGS

FIG. 1 is a schematic showing the gene transfer and gene editing mechanism of an rAAV as disclosed herein.

FIGS. 2A-2D are schematics showing the plasmid maps of PAH-006m (FIG. 2A), PAH-006m-LP-1 (FIG. 2B), PAH-032h (FIG. 2C), and hPAH-hI1C-032-LP1-SD3 (FIG. 2D).

FIG. 3 is a plot showing the effect of PAH-006m on blood Phe levels in PAH^{enu2} mice.

FIG. 4 is a plot showing on-target insertion at the mouse PAH locus following administration of PAH-006m.

FIG. 5 is a plot showing the effect of PAH-006m on mRNA expression relative to mouse GAPDH.

FIG. 6 is a plot showing on-target insertion in FRG® mice following administration of PAH-032h as measured by species specific ddPCR editing assays.

FIG. 7 is a plot showing editing of human hepatocytes in FRG mice as measured by quantitative next generation sequencing assays.

FIGS. 8A-8C are plots showing the effect of PAH-006m-LP-1 on blood Phe concentration in PAH^{enu2} mice over time. FIG. 8A shows the full data set, while FIG. 8B shows the data over a certain range in time with a reduced y-axis scale in order to show differences between the dose levels. FIG. 8C shows the on-target integration of PAH-006m-LP-1 in PAH^{enu2} mice, as measured by next generation sequencing.

FIGS. 9A-9L are plots showing the efficacy of the gene transfer/gene editing vector PAH-006m-LP-1 and an episomal transgene expression vector. FIGS. 9A and 9B are plots showing the effect of the episomal transgene expression vector and PAH-006m-LP-1, respectively, packaged in AAVHSC15 capsid, on the serum Phe levels of PAH^{enu2} mice, up to 12 weeks post-dosing at the indicated doses. FIGS. 9C and 9D are plots showing the effect of the episomal transgene expression vector and PAH-006m-LP-1, respectively, packaged in AAVHSC15 capsid, on the serum Tyr levels of PAH^{enu2} mice, up to 12 weeks post-dosing at the indicated doses. FIGS. 9E and 9F are plots showing the effect of the episomal transgene expression vector and PAH-006m-LP-1, respectively, packaged in AAVHSC15 capsid, on the level of vector genomes detected per ug of genomic DNA in PAH^{enu2} mice, at the indicated doses. FIGS. 9G and 9H are plots showing the effect of the episomal transgene expression vector and PAH-006m-LP-1, respectively, packaged in AAVHSC15 capsid, on the level of mRNA expression per 10 ng of total RNA detected in PAH^{enu2} mice, at the indicated doses.

FIG. 9I is a plot showing the level of on-target integration presented by the number of viral genomes per allele detected by ddPCR across various doses as indicated, at 12 weeks post-dosing of vector packaged in AAVHSC15 as indicated. FIG. 9J is a plot showing the frequency of on-target insertion detected across various doses as indicated, at 12 weeks post-dosing of vector packaged in AAVHSC15 as indicated. FIGS. 9K and 9L are plots showing time courses out to 42 weeks (FIG. 9K) or 40 weeks (FIG. 9L) post-dosing of PAH^{enu2} mice with vector and dosage as indicated, of the results of the effect of a partial hepatectomy (PHx) on serum Phe levels. PHx or sham surgery was performed at around 2 weeks post-dosing.

FIGS. 10A-10P are plots showing dose range differences between the mouse-specific gene transfer/gene editing AAV vector (PAH-006m-LP-1; “mouse design”), and the human-specific gene transfer/gene editing AAV vector (hPAH-hI1C-032-LP1-SD3; “human design”) in 4-week (FIGS. 10A-10H) or 10-week-old (FIGS. 10I-10P) PAH^{enu2} mice administered the vector packaged in AAVHSC15 capsid as indicated, at the dosage as indicated.

FIGS. 11A-11K are plots showing the effect the age of PAH^{enu2} mice has on the response to a single dose of the mouse-specific gene transfer/gene editing AAV vector (PAH-006m-LP-1; “mouse design”), or the human-specific gene transfer/gene editing AAV vector (hPAH-hI1C-032-LP1-SD3; “human design”). Serum Phe and Tyr levels (FIGS. 11A-11D) and vector genome and mRNA levels (FIGS. 11E-11K) were examined over time for the various dosing groups set forth in Table 2.

FIGS. 12A-12C are plots showing the kinetics and durability of integration over time, of PAH^{enu2} mice administered a mouse-specific gene transfer/gene editing AAV vector (PAH-006m-LP-1) packaged in AAVHSC15 capsid at a dose of 1E14 vg/kg. FIGS. 12A and 12B are plots showing the effect of PAH-006m-LP-1 packaged in AAVHSC15 capsid on the level of vector genomes detected per ug of genomic DNA, and the level of mRNA expression detected per 10 ng of total RNA in PAH^{enu2} mice, respectively, over time. FIG. 12C is a plot showing the frequency of on-target insertion detected at various time points post-dosing of PAH-006m-LP-1 packaged in AAVHSC15 capsid. FIGS. 12D and 12E are graphs showing the effect on serum Phe (FIG. 12D) and Tyr (FIG. 12E) in PAH^{enu2} mice administered PAH-006m-LP-1 packaged in AAVHSC15 capsid at a dose of 1E14 vg/kg, up to 42 weeks post-injection.

FIGS. 13A and 13B are plots showing the effect of hPAH-hI1C-032-LP1-SD3 packaged in AAVHSC15 on the level of vector genomes detected per ug of genomic DNA, and the level of mRNA expression detected per 10 ng of total RNA in human hepatocytes isolated from HuLiv mice, respectively, at the indicated doses and timepoints. FIG. 13C is a plot showing the frequency of on-target insertion detected in human hepatocytes isolated from HuLiv mice at various time points post-dosing of hPAH-hI1C-032-LP1-SD3 packaged in AAVHSC15, at the indicated doses.

FIGS. 14A-14D are plots showing the amount of integration at predicted off-target integration sites detected in genomic DNA isolated from HuLiv mice administered the human-specific gene transfer/gene editing vector, hPAH-hI1C-032-LP1-SD3, packaged in AAVHSC15 capsid.

### DETAILED DESCRIPTION

Provided herein are recombinant adeno-associated virus (rAAV) compositions that can restore PAH gene function in cells, and methods for using the same to treat diseases associated with reduction of PAH gene function (e.g., PKU). Also provided are nucleic acids, vectors, packaging systems, and methods for making the adeno-associated virus compositions.

### I. DEFINITIONS

As used herein, the terms “recombinant adeno-associated virus” or “rAAV” refers to an AAV comprising a genome lacking functional rep and cap genes.

As used herein, the term “PAH gene” refers to the phenylalanine hydroxylase (PAH) gene, including but not limited to the coding regions, exons, introns, 5′ UTR, 3′ UTR, and transcriptional regulatory regions of the PAH gene. The human PAH gene is identified by Entrez Gene ID 5053. An exemplary nucleotide sequence of a PAH mRNA is provided as SEQ ID NO: 53. An exemplary amino acid sequence of a PAH polypeptide is provided as SEQ ID NO: 33. In certain embodiments, the PAH gene is a variant PAH gene. Variant PAH genes are known to those of skill in the art and may comprise one or more nucleotide differences as compared to the reference human genome. In certain embodiments, a variant PAH gene is a common variant observed in the general populace. For example, variant rs1522296 comprises a single nucleotide difference from the reference human genome at the genomic location in the human genome build Hg38 at chr12: 103310787. This variant is seen in >30% of the global population and has no known association to either changes in PAH expression or disease risk.

As used herein, the term “rAAV genome” refers to a recombinant AAV genome that is capable of integrating an editing element (e.g., one or more nucleotides or an internucleotide bond) via homologous recombination into a target locus to correct a genetic defect in a PAH gene. In certain embodiments, the target locus is in the human PAH gene. The skilled artisan will appreciate that the portion of an rAAV genome comprising the 5′ homology arm, editing element, and 3′ homology arm can be in the sense or antisense orientation relative to the target locus (e.g., the human PAH gene).

As used herein, the term “editing element” refers to the portion of an rAAV genome that when integrated at a target locus modifies the target locus. An editing element can mediate insertion, deletion, or substitution of one or more nucleotides at the target locus.

As used herein, the term “target locus” refers to a region of a chromosome or an internucleotide bond (e.g., a region or an internucleotide bond of the human PAH gene) that is modified by an editing element.

As used herein, the term “homology arm” refers to a portion of an rAAV genome positioned 5′ or 3′ of an editing element that is substantially identical to the genome flanking a target locus. In certain embodiments, the target locus is in a human PAH gene, and the homology arm comprises a sequence substantially identical to the genome flanking the target locus.

As used herein, the term “AAV capsid protein” refers to an AAV VP1, VP2, or VP3 capsid protein. As used herein, the term “Clade F capsid protein” refers to an AAV VP1, VP2, or VP3 capsid protein that comprises an amino acid sequence having at least 90% identity with the VP1, VP2, or VP3 amino acid sequences set forth, respectively, in amino acids 1-736, 138-736, and 203-736 of SEQ ID NO: 1 herein.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches and does not consider the degree of similarity of amino acids to one another. Note that only internal gaps are included in the length, not gaps at the sequence ends.

As used herein, the term “a disease or disorder associated with a PAH gene mutation” refers to any disease or disorder caused by, exacerbated by, or genetically linked with variation of a PAH gene. In certain embodiments, the disease or disorder associated with a PAH gene mutation is phenylketonuria (PKU).

As used herein, the term “silently altered” refers to alteration of a coding sequence or a stuffer-inserted coding sequence of a gene (e.g., by nucleotide substitution) without changing the amino acid sequence of the polypeptide encoded by the coding sequence or stuffer-inserted coding sequence. Codon alteration can be conducted by any method known in the art (e.g., as described in Mauro & Chappell (2014) Trends Mol Med. 20 (11): 604-13, which is incorporated by reference herein in its entirety). Such silent alteration is advantageous in that it reduces the likelihood of integration of the rAAV genome into loci of other genes or pseudogenes paralogous to the target gene. Such silent alteration also reduces the homology between the editing element and the target gene, thereby reducing undesired integration mediated by the editing element rather than by a homology arm.

As used herein, the term “coding sequence” refers to the portion of a complementary DNA (cDNA) that encodes a polypeptide, starting at the start codon and ending at the stop codon. A gene may have one or more coding sequences due to alternative splicing and/or alternative translation initiation. A coding sequence may either be wild-type or silently altered. An exemplary wild-type PAH coding sequence is set forth in SEQ ID NO: 53.

As used herein, the term “polyadenylation sequence” refers to a DNA sequence that when transcribed into RNA constitutes a polyadenylation signal sequence. The polyadenylation sequence can be native (e.g., from the PAH gene) or exogenous. The exogenous polyadenylation sequence can be a mammalian or a viral polyadenylation sequence (e.g., an SV40 polyadenylation sequence).

As used herein, the term “intron element” refers to a cis-acting nucleotide sequence, for example, a DNA sequence, that regulates (e.g., controls, increases, or reduces) expression of a transgene. In certain embodiments, an intron element is a modified intron, e.g., a synthetic intron sequence. In certain embodiments, an intron element is an exogenous intron element and is derived from an intron exogenous to the transgene it may regulate. In certain embodiments, an intron element comprises a modified splice acceptor and/or splice donor resulting in more robust splicing activity. While not wishing to be bound by theory, it is hypothesized that introns can increase transgene expression, for example, by reducing transcriptional silencing and enhancing mRNA export from the nucleus to the cytoplasm. A skilled worker will appreciate that synthetic intron sequences can be designed to mediate RNA splicing by introducing any consensus splicing motifs known in the art (e.g., in Sibley et al. (2016) Nature Reviews Genetics, 17, 407-21, which is incorporated by reference herein in its entirety). Exemplary intron sequences are provided in Lu et al. (2013) Molecular Therapy 21 (5): 954-63, and Lu et al. (2017) Hum. Gene Ther. 28 (1): 125-34, which are incorporated by reference herein in their entirety.

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE relies on one or more trans-acting molecules, such as transcription factors, to regulate transcription. Thus, one TRE may regulate transcription in different ways when it is in contact with different trans-acting molecules, for example, when it is in different types of cells. A TRE may comprise one or more promoter elements and/or enhancer elements. A skilled artisan would appreciate that the promoter and enhancer elements in a gene may be close in location, and the term “promoter” may refer to a sequence comprising a promoter element and an enhancer element. Thus, the term “promoter” does not exclude an enhancer element in the sequence. The promoter and enhancer elements do not need to be derived from the same gene or species, and the sequence of each promoter or enhancer element may be either identical or substantially identical to the corresponding endogenous sequence in the genome.

As used herein, the term “operably linked” is used to describe the connection between a TRE and a coding sequence to be transcribed. Typically, gene expression is placed under the control of a TRE comprising one or more promoter and/or enhancer elements. The coding sequence is “operably linked” to the TRE if the transcription of the coding sequence is controlled or influenced by the TRE. The promoter and enhancer elements of the TRE may be in any orientation and/or distance from the coding sequence, as long as the desired transcriptional activity is obtained. In certain embodiments, the TRE is upstream from the coding sequence.

In the instant disclosure, nucleotide positions in a PAH gene are specified relative to the first nucleotide of the start codon. The first nucleotide of a start codon is position 1; the nucleotides 5′ to the first nucleotide of the start codon have negative numbers; the nucleotides 3′ to the first nucleotide of the start codon have positive numbers. As used herein, nucleotide 1 of the human PAH gene is nucleotide 5,473 of the NCBI Reference Sequence: NG_008690.1, and nucleotide-1 of the human PAH gene is nucleotide 5,472 of the NCBI Reference Sequence: NG_008690.1.

In the instant disclosure, exons and introns in a PAH gene are specified relative to the exon encompassing the first nucleotide of the start codon, which is nucleotide 5473 of the NCBI Reference Sequence: NG_008690.1. The exon encompassing the first nucleotide of the start codon is exon 1. Exons 3′ to exon 1 are from 5′ to 3′: exon 2, exon 3, etc. Introns 3′ to exon 1 are from 5′ to 3′: intron 1, intron 2, etc. Accordingly, the PAH gene comprises from 5′ to 3′: exon 1, intron 1, exon 2, intron 2, exon 3, etc. As used herein, exon 1 of the human PAH gene is nucleotides 5001-5532 of the NCBI Reference Sequence: NG_008690.1, and intron 1 of the human PAH gene is nucleotides 5533-9704 of the NCBI Reference Sequence: NG_008690.1.

As used herein, the term “integration” refers to introduction of an editing element into a target locus (e.g., of a PAH gene) by homologous recombination between an rAAV genome and the target locus. Integration of an editing element can result in substitution, insertion and/or deletion of one or more nucleotides in a target locus (e.g., of a PAH gene).

As used herein, the term “integration efficiency of the editing element into the target locus” refers to the percentage of cells in a transduced population in which integration of the editing element into the target locus has occurred.

As used herein, the term “allelic frequency of integration of the editing element into the target locus” refers to the percentage of alleles in a population of transduced cells in which integration of the editing element into the target locus has occurred.

As used herein, the term “standard AAV administration conditions” refers to transduction of human hepatocytes implanted into a mouse following hepatocyte ablation, wherein the AAV is administered intravenously at a dose of 1×10¹³ vector genomes per kilogram of body weight.

As used herein, the term “effective amount” in the context of the administration of an AAV to a subject refers to the amount of the AAV that achieves a desired prophylactic or therapeutic effect.

As used herein, the term “about” or “approximately” when referring to a measurable value, such as the expression level of an IDS protein, encompasses variations of ±20% or ±10%, ±5%, ±1%, or ±0.1% of a given value or range, as are appropriate to perform the methods disclosed herein.

### II. ADENO-ASSOCIATED VIRUS COMPOSITIONS

In one aspect, provided herein are novel rAAV compositions useful for restoring PAH expression in cells with reduced or otherwise defective PAH gene function. Such rAAV compositions are highly efficient at editing the genome of cells (e.g., liver cells) in a subject to express PAH under the control of a liver-specific promoter, and do not require cleavage of the genome at the target locus by the action of an exogenous nuclease (e.g., a meganuclease, a zinc finger nuclease, a transcriptional activator-like nuclease (TALEN), or an RNA-guided nuclease such as a Cas9) to facilitate such editing. Accordingly, in certain embodiments, the rAAV compositions disclosed herein do not comprise or require an exogenous nuclease or a nucleotide sequence that encodes an exogenous nuclease.

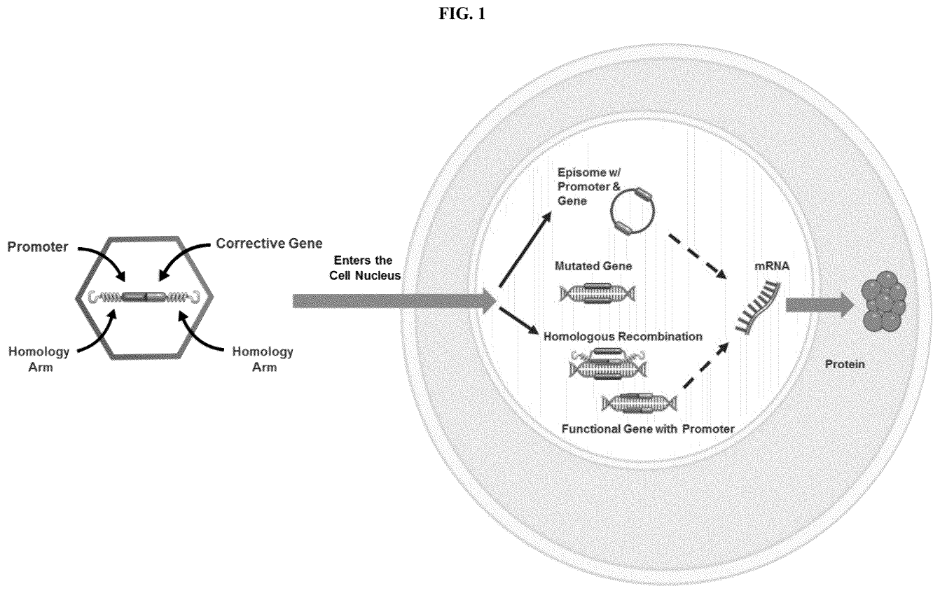

In certain embodiments, the rAAV disclosed herein comprises: (a) an AAV capsid comprising an AAV capsid protein (e.g., an AAV Clade F capsid protein); and (b) an rAAV genome comprising: (i) an editing element for editing a target locus in a PAH gene, comprising at least a portion of a PAH coding sequence operably linked to a transcriptional regulatory element; (ii) a 5′ homology arm nucleotide sequence position 5′ of the editing element, having homology to a first genomic region 5′ to the target locus; and (iii) a 3′ homology arm nucleotide sequence positioned 3′ of the editing element, having homology to a second genomic region 3′ to the target locus. In certain embodiments, the rAAV disclosed herein has the potential to express a PAH transgene both via episomal expression and through insertion of the editing element into the genome at the target locus in the PAH gene (see, FIG. 1). In certain embodiments, the rAAV disclosed herein allows for the expression of PAH to be maintained throughout the period of hepatic growth, during which episomal expression may be lost. Accordingly, the rAAV compositions are particularly useful for treating juvenile PKU.

A capsid protein from any capsid known in the art can be used in the rAAV compositions disclosed herein, including, without limitation, a capsid protein from an AAV1, AAV2, AAV3, AAV4, AAV5, AAV6, AAV7, AAV8, or AAV9 serotype. For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17, wherein: the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 203-736 of SEQ ID NO: 1, 2, 3, 4, 5, 6, 7, 8, 9, 10, 11, 12, 13, 15, 16, or 17.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17, wherein: the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 138-736 of SEQ ID NO: 1, 2, 3, 4, 5, 6, 7, 8, 9, 10, 11, 12, 13, 15, 16, or 17.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17, wherein: the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 2 of SEQ ID NO: 16 is T, and the amino acid in the capsid protein corresponding to amino acid 312 of SEQ ID NO: 16 is Q. In certain embodiments, the amino acid in the capsid protein corresponding to amino acid 65 of SEQ ID NO: 16 is I, and the amino acid in the capsid protein corresponding to amino acid 626 of SEQ ID NO: 16 is Y. In certain embodiments, the amino acid in the capsid protein corresponding to amino acid 77 of SEQ ID NO: 16 is R, and the amino acid in the capsid protein corresponding to amino acid 690 of SEQ ID NO: 16 is K. In certain embodiments, the amino acid in the capsid protein corresponding to amino acid 119 of SEQ ID NO: 16 is L, and the amino acid in the capsid protein corresponding to amino acid 468 of SEQ ID NO: 16 is S. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 1-736 of SEQ ID NO: 1, 2, 3, 4, 5, 6, 7, 8, 9, 10, 11, 12, 13, 15, 16, or 17.

In certain embodiments, the AAV capsid comprises two or more of: (a) a capsid protein comprising the amino acid sequence of amino acids 203-736 of SEQ ID NO: 1, 2, 3, 4, 6, 7, 10, 11, 12, 13, 15, 16, or 17; (b) a capsid protein comprising the amino acid sequence of amino acids 138-736 of SEQ ID NO: 1, 2, 3, 4, 5, 6, 7, 9, 10, 11, 12, 13, 15, 16, or 17; and (c) a capsid protein comprising the amino acid sequence of amino acids 1-736 of SEQ ID NO: 1, 2, 3, 4, 5, 6, 7, 8, 9, 10, 11, 12, 13, 15, 16, or 17. In certain embodiments, the AAV capsid comprises: (a) a capsid protein having an amino acid sequence consisting of amino acids 203-736 of SEQ ID NO: 1, 2, 3, 4, 6, 7, 10, 11, 12, 13, 15, 16, or 17; (b) a capsid protein having an amino acid sequence consisting of amino acids 138-736 of SEQ ID NO: 1, 2, 3, 4, 5, 6, 7, 9, 10, 11, 12, 13, 15, 16, or 17; and (c) a capsid protein having an amino acid sequence consisting of amino acids 1-736 of SEQ ID NO: 1, 2, 3, 4, 5, 6, 7, 8, 9, 10, 11, 12, 13, 15, 16, or 17.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 8; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 8; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 8. In certain embodiments, the AAV capsid comprises one or more of: (a) a capsid protein comprising the amino acid sequence of amino acids 203-736 of SEQ ID NO: 8; (b) a capsid protein comprising the amino acid sequence of amino acids 138-736 of SEQ ID NO: 8; and (c) a capsid protein comprising the amino acid sequence of amino acids 1-736 of SEQ ID NO: 8. In certain embodiments, the AAV capsid comprises two or more of: (a) a capsid protein comprising the amino acid sequence of amino acids 203-736 of SEQ ID NO: 8; (b) a capsid protein comprising the amino acid sequence of amino acids 138-736 of SEQ ID NO: 8; and (c) a capsid protein comprising the amino acid sequence of amino acids 1-736 of SEQ ID NO: 8. In certain embodiments, the AAV capsid comprises: (a) a capsid protein having an amino acid sequence consisting of amino acids 203-736 of SEQ ID NO: 8; (b) a capsid protein having an amino acid sequence consisting of amino acids 138-736 of SEQ ID NO: 8; and (c) a capsid protein having an amino acid sequence consisting of amino acids 1-736 of SEQ ID NO: 8.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 11; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 11; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 11. In certain embodiments, the AAV capsid comprises one or more of: (a) a capsid protein comprising the amino acid sequence of amino acids 203-736 of SEQ ID NO: 11; (b) a capsid protein comprising the amino acid sequence of amino acids 138-736 of SEQ ID NO: 11; and (c) a capsid protein comprising the amino acid sequence of amino acids 1-736 of SEQ ID NO: 11. In certain embodiments, the AAV capsid comprises two or more of: (a) a capsid protein comprising the amino acid sequence of amino acids 203-736 of SEQ ID NO: 11; (b) a capsid protein comprising the amino acid sequence of amino acids 138-736 of SEQ ID NO: 11; and (c) a capsid protein comprising the amino acid sequence of amino acids 1-736 of SEQ ID NO: 11. In certain embodiments, the AAV capsid comprises: (a) a capsid protein having an amino acid sequence consisting of amino acids 203-736 of SEQ ID NO: 11; (b) a capsid protein having an amino acid sequence consisting of amino acids 138-736 of SEQ ID NO: 11; and (c) a capsid protein having an amino acid sequence consisting of amino acids 1-736 of SEQ ID NO: 11.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 13; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 13; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 13. In certain embodiments, the AAV capsid comprises one or more of: (a) a capsid protein comprising the amino acid sequence of amino acids 203-736 of SEQ ID NO: 13; (b) a capsid protein comprising the amino acid sequence of amino acids 138-736 of SEQ ID NO: 13; and (c) a capsid protein comprising the amino acid sequence of amino acids 1-736 of SEQ ID NO: 13. In certain embodiments, the AAV capsid comprises two or more of: (a) a capsid protein comprising the amino acid sequence of amino acids 203-736 of SEQ ID NO: 13; (b) a capsid protein comprising the amino acid sequence of amino acids 138-736 of SEQ ID NO: 13; and (c) a capsid protein comprising the amino acid sequence of amino acids 1-736 of SEQ ID NO: 13. In certain embodiments, the AAV capsid comprises: (a) a capsid protein having an amino acid sequence consisting of amino acids 203-736 of SEQ ID NO: 13; (b) a capsid protein having an amino acid sequence consisting of amino acids 138-736 of SEQ ID NO: 13; and (c) a capsid protein having an amino acid sequence consisting of amino acids 1-736 of SEQ ID NO: 13.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 203-736 of SEQ ID NO: 16; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 138-736 of SEQ ID NO: 16; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 1-736 of SEQ ID NO: 16. In certain embodiments, the AAV capsid comprises one or more of: (a) a capsid protein comprising the amino acid sequence of amino acids 203-736 of SEQ ID NO: 16; (b) a capsid protein comprising the amino acid sequence of amino acids 138-736 of SEQ ID NO: 16; and (c) a capsid protein comprising the amino acid sequence of amino acids 1-736 of SEQ ID NO: 16. In certain embodiments, the AAV capsid comprises two or more of: (a) a capsid protein comprising the amino acid sequence of amino acids 203-736 of SEQ ID NO: 16; (b) a capsid protein comprising the amino acid sequence of amino acids 138-736 of SEQ ID NO: 16; and (c) a capsid protein comprising the amino acid sequence of amino acids 1-736 of SEQ ID NO: 16. In certain embodiments, the AAV capsid comprises: (a) a capsid protein having an amino acid sequence consisting of amino acids 203-736 of SEQ ID NO: 16; (b) a capsid protein having an amino acid sequence consisting of amino acids 138-736 of SEQ ID NO: 16; and (c) a capsid protein having an amino acid sequence consisting of amino acids 1-736 of SEQ ID NO: 16.

rAAV genomes useful in the AAV compositions disclosed herein generally comprise: (i) an editing element for editing a target locus in a PAH gene, comprising at least a portion of a PAH coding sequence operably linked to a transcriptional regulatory element; (ii) a 5′ homology arm nucleotide sequence position 5′ of the editing element, having homology to a first genomic region 5′ to the target locus; and (iii) a 3′ homology arm nucleotide sequence positioned 3′ of the editing element, having homology to a second genomic region 3′ to the target locus. In certain embodiments, the rAAV genome comprises a 5′ inverted terminal repeat (5′ ITR) nucleotide sequence 5′ of the 5′ homology arm nucleotide sequence, and a 3′ inverted terminal repeat (3′ ITR) nucleotide sequence 3′ of the 3′ homology arm nucleotide sequence.

Editing elements used in the rAAV genomes disclosed herein can mediate insertion, deletion, or substitution of one or more nucleotides at the target locus.

In certain embodiments, when correctly integrated by homologous recombination at the target locus, the editing element inserts a nucleotide sequence comprising at least a portion of a PAH coding sequence into a PAH gene. In certain embodiments, the editing element comprises a PAH coding sequence (e.g., a complete PAH coding sequence).

In certain embodiments, the PAH coding sequence encodes a wild-type PAH polypeptide (e.g., having the amino acid sequence set forth in SEQ ID NO: 33). In certain embodiments, the PAH coding sequence is wild-type (e.g., comprising the nucleotide sequence set forth in SEQ ID NO: 53). In certain embodiments, the PAH coding sequence is silently altered to be less than 100% (e.g., less than 95%, 90%, 85%, 80%, 75%, 70%, 65%, 60%, 55%, or 50%) identical to the corresponding exons of the wild-type PAH gene. In certain embodiments, the PAH coding sequence comprises or consists of a nucleotide sequence that is at least 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence set forth in SEQ ID NO: 28, 63, or 83. In certain embodiments, the PAH coding sequence comprises the nucleotide sequence set forth in SEQ ID NO: 28. In certain embodiments, the PAH coding sequence consists of the nucleotide sequence set forth in SEQ ID NO: 28. In certain embodiments, the PAH coding sequence comprises the nucleotide sequence set forth in SEQ ID NO: 63. In certain embodiments, the PAH coding sequence consists of the nucleotide sequence set forth in SEQ ID NO: 63. In certain embodiments, the PAH coding sequence comprises the nucleotide sequence set forth in SEQ ID NO: 83. In certain embodiments, the PAH coding sequence consists of the nucleotide sequence set forth in SEQ ID NO: 83.

In certain embodiments, rAAV genomes useful in the AAV compositions disclosed herein comprise a transcriptional regulatory element (TRE) operably linked to at least a portion of a PAH coding sequence. In certain embodiments, rAAV genomes useful in the AAV compositions disclosed herein comprise from 5′ to 3′: a TRE, and the at least a portion of a PAH coding sequence.

The rAAV genome can be used to express PAH in any mammalian cells (e.g., human cells). Thus, the TRE can be active in any mammalian cells (e.g., human cells). In certain embodiments, the TRE is active in a broad range of human cells. Such TREs may comprise constitutive promoter and/or enhancer elements including cytomegalovirus (CMV) promoter/enhancer (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 54, 55, or 56), SV40 promoter, chicken ACTB promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 47 or 57), JeT promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 58), smCBA promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 59), human elongation factor 1 alpha (EF1α) promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 39), minute virus of mouse (MVM) intron which comprises transcription factor binding sites (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 61), human phosphoglycerate kinase (PGK1) promoter, human ubiquitin C (Ubc) promoter, human beta actin promoter, human neuron-specific enolase (ENO2) promoter, human beta-glucuronidase (GUSB) promoter, a rabbit beta-globin element (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 41 or 62), human calmodulin 1 (CALM1) promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 44), and/or human Methyl-CpG Binding Protein 2 (MeCP2) promoter. Any of these TREs can be combined in any order to drive efficient transcription. For example, an rAAV genome may comprise a CMV enhancer, a CBA promoter, and the splice acceptor from exon 3 of the rabbit beta-globin gene, collectively called a CAG promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 42 or 64). For example, an rAAV genome may comprise a hybrid of CMV enhancer and CBA promoter followed by a splice donor and splice acceptor, collectively called a CASI promoter region (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 48 or 65).

Alternatively, the TRE may be a tissue-specific TRE, i.e., it is active in specific tissue(s) and/or organ(s). A tissue-specific TRE comprises one or more tissue-specific promoter and/or enhancer elements, and optionally one or more constitutive promoter and/or enhancer elements. A skilled artisan would appreciate that tissue-specific promoter and/or enhancer elements can be isolated from genes specifically expressed in the tissue by methods well known in the art.

In certain embodiments, the TRE is liver-specific (e.g., hepatocyte-specific). Exemplary liver-specific TREs may comprise one or more elements selected from the group consisting of human albumin promoter, human transthyretin (TTR) promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 66), human APOE/C-I hepatic control region (HCR) 1 or 2 (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 25 or 68), human APOH promoter, and human SERPINA1 (hAAT) promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 26, 69 or 70) or a hepatic specific regulatory module thereof (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 71). In certain embodiments, an hAAT promoter region comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 72. More liver-specific promoter elements are disclosed in WO 2009/130208 and Kramer et al. Molecular Therapy (2003) 7, 375-385, which are incorporated by reference herein in their entirety.

In certain embodiments, the TRE is kidney-specific (e.g., renal epithelial cell-specific). Exemplary kidney-specific TREs may comprise one or more elements selected from the group consisting of human nephrin promoter, human parathyroid hormone receptor promoter, human uromodulin promoter, and human SLC12A1 promoter. In certain embodiments, the TRE is brain-specific (e.g., neuron-specific, glial cell-specific, astrocyte-specific, oligodendrocyte-specific, microglia-specific and/or central nervous system-specific). Exemplary brain-specific TREs may comprise one or more elements selected from the group consisting of human glial fibrillary acidic protein (GFAP) promoter and human synapsin 1 (SYN1) promoter. More brain-specific promoter elements are disclosed in WO 2016/100575A1, which is incorporated by reference herein in its entirety.

In certain embodiments, the rAAV genome comprises two or more TRES, optionally comprising at least one of the TREs disclosed above. A skilled person in the art would appreciate that any of these TREs can be combined in any order, and combinations of a constitutive TRE and a tissue-specific TRE can drive efficient and tissue-specific transcription. For example, in certain embodiments, the rAAV genome comprises a human HCR1 (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 25 or 68) and a human EF-1α promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 39), optionally wherein the human HCR1 is 5′ to the human EF-1α promoter. In certain embodiments, the rAAV genome comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence nucleotide set forth in SEQ ID NO: 60.

Similarly, combinations of two or more tissue-specific TREs can drive efficient and tissue-specific transcription. For example, in certain embodiments, the rAAV genome comprises a human HCR1 (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 25) and a hAAT promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 26), optionally wherein the human HCR1 is 5′ to the hAAT promoter. In certain embodiments, the rAAV genome comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 27. In certain embodiments, the rAAV genome comprises a human HCR1 (e.g., comprising the nucleotide sequence set forth in SEQ ID NO: 25) and a hAAT promoter (e.g., comprising the nucleotide sequence set forth in SEQ ID NO: 26), optionally wherein the human HCR1 is 5′ to the hAAT promoter. In certain embodiments, the rAAV genome comprises the nucleotide sequence set forth in SEQ ID NO: 27.

In certain embodiments, the rAAV genome comprises a hepatic specific regulatory module of hAAT promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 71) and a human TTR promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 66), optionally wherein the hepatic specific regulatory module is 5′ to the human TTR promoter. In certain embodiments, the rAAV genome comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 67. In certain embodiments, the rAAV genome comprises a hepatic specific regulatory module of hAAT promoter (e.g., comprising the nucleotide sequence set forth in SEQ ID NO: 71) and a human TTR promoter (e.g., comprising the nucleotide sequence set forth in SEQ ID NO: 66), optionally wherein the hepatic specific regulatory module is 5′ to the human TTR promoter. In certain embodiments, the rAAV genome comprises the nucleotide sequence set forth in SEQ ID NO: 67.

In certain embodiments, the rAAV genome further comprises an intron element 5′ to the at least a portion of a PAH coding sequence. Such intron elements can increase transgene expression, for example, by reducing transcriptional silencing and enhancing mRNA export from the nucleus to the cytoplasm. In certain embodiments, the rAAV genome comprises from 5′ to 3′: a TRE, an intron element, and the at least a portion of a PAH coding sequence.

The intron element can comprise at least a portion of a native intron sequence of the PAH gene, or the intron element can be an exogenous intron element (e.g., comprising at least an intron sequence from a different species or a different gene from the same species, and/or a synthetic intron sequence). In certain embodiments, the intron element is an exogenous intron element comprising at least a portion of an intron sequence from a different species. In certain embodiments, the intron element is an exogenous intron element comprising at least a portion of an intron sequence from a different gene from the same species. In certain embodiments, the intron element is an exogenous intron element comprising a synthetic intron sequence. In certain embodiments, the intron element is an exogenous intron element comprising a combination of at least an intron sequence from a different species or a different gene from the same species, and/or a synthetic intron sequence.

A skilled worker will appreciate that intron elements can be designed to mediate RNA splicing by introducing any consensus splicing motifs known in the art (e.g., in Sibley et al. (2016) Nature Reviews Genetics, 17, 407-21, which is incorporated by reference herein in its entirety). Exemplary intron sequences are provided in Lu et al. (2013) Molecular Therapy 21(5): 954-63, and Lu et al. (2017) Hum. Gene Ther. 28 (1): 125-34, which are incorporated by reference herein in their entirety.

In certain embodiments, the rAAV genome comprises an exogenous intron element. In certain embodiments, the rAAV comprises an SV40 intron element (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 29) or a minute virus of mouse (MVM) intron (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 61). In certain embodiments, the rAAV genome comprises an SV40 intron element (e.g., comprising the nucleotide sequence set forth in SEQ ID NO: 29) or a minute virus of mouse (MVM) intron element (e.g., comprising the nucleotide sequence set forth in SEQ ID NO: 61).

In certain embodiments, the rAAV genome disclosed herein further comprises a transcription terminator (e.g., a polyadenylation sequence). In certain embodiments, the transcription terminator is 3′ to the at least a portion of a PAH coding sequence. The transcription terminator may be any sequence that effectively terminates transcription, and a skilled artisan would appreciate that such sequences can be isolated from any genes that are expressed in the cell in which transcription of the at least a portion of a PAH coding sequence is desired. In certain embodiments, the transcription terminator comprises a polyadenylation sequence. In certain embodiments, the polyadenylation sequence is identical or substantially identical to the endogenous polyadenylation sequence of the human PAH gene. In certain embodiments, the polyadenylation sequence is an exogenous polyadenylation sequence. In certain embodiments, the polyadenylation sequence is an SV40 polyadenylation sequence (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 31, 34, or 35, or a nucleotide sequence complementary thereto). In certain embodiments, the polyadenylation sequence comprises the nucleotide sequence set forth in SEQ ID NO: 31. In certain embodiments, the polyadenylation sequence consists of the nucleotide sequence set forth in SEQ ID NO: 31.

In certain embodiments, the rAAV genome comprises from 5′ to 3′: a TRE, an intron element, at least a portion of a PAH coding sequence, and a polyadenylation sequence. In certain embodiments, the TRE has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 25-27, 30, 36, 39, 40-42, 44, 46-49, 54-60, or 62-72; the intron element has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 29 or 61; the at least a portion of a PAH coding sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 28; and/or the polyadenylation sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 31, 34, or 35. In certain embodiments, the TRE comprises a nucleotide sequence selected from the group consisting of SEQ ID NOs: 25-27, 30, 36, 39, 40-42, 44, 46-49, 54-60, and 62-72; the intron element comprises a nucleotide sequence selected from the group consisting of SEQ ID NOs: 29 and 61; the at least a portion of a PAH coding sequence comprises the nucleotide sequence set forth in SEQ ID NO: 28; and/or the polyadenylation sequence comprises a nucleotide sequence selected from the group consisting of SEQ ID NOs: 31, 34, and 35.

In certain embodiments, the TRE comprises or consists of the nucleotide sequence set forth in SEQ ID NO: 25, 26, or 27; the intron element comprises or consists of the nucleotide sequence set forth in SEQ ID NO: 29; the at least a portion of a PAH coding sequence comprises or consists of the nucleotide sequence set forth in SEQ ID NO: 28; and/or the polyadenylation sequence comprises or consists of the nucleotide sequence set forth in SEQ ID NO: 31. In certain embodiments, the TRE comprises from 5′ to 3′ the nucleotide sequence set forth in SEQ ID NO: 25, and the nucleotide sequence set forth in SEQ ID NO: 26 (e.g., the TRE comprises the nucleotide sequence set forth in SEQ ID NO: 27); the intron element comprises the nucleotide sequence set forth in SEQ ID NO: 29; the at least a portion of a PAH coding sequence comprises the nucleotide sequence set forth in SEQ ID NO: 28; and/or the polyadenylation sequence comprises the nucleotide sequence set forth in SEQ ID NO: 31.

Homology arms used in the rAAV genomes disclosed herein can be directed to any region of the PAH gene or a gene nearby on the genome. The precise identity and positioning of the homology arms are determined by the identity of the editing element and/or the target locus.

Homology arms employed in the rAAV genomes disclosed herein are substantially identical to the genome flanking a target locus (e.g., a target locus in a PAH gene). In certain embodiments, the 5′ homology arm has at least about 90% (e.g., at least about 91%, 92%, 93%, 94%, 95%, 96%, 97%, 98%, 99%, or 99.5%) nucleotide sequence identity to a first genomic region 5′ to the target locus. In certain embodiments, the 5′ homology arm has 100% nucleotide sequence identity to the first genomic region. In certain embodiments, the 3′ homology arm has at least about 90% (e.g., at least about 91%, 92%, 93%, 94%, 95%, 96%, 97%, 98%, 99%, or 99.5%) nucleotide sequence identity to a second genomic region 3′ to the target locus. In certain embodiments, the 3′ homology arm has 100% nucleotide sequence identity to the second genomic region. In certain embodiments, the 5′ and 3′ homology arms are each at least about 90% (e.g., at least about 91%, 92%, 93%, 94%, 95%, 96%, 97%, 98%, 99%, or 99.5%) identical to the first and second genomic regions flanking the target locus (e.g., a target locus in the PAH gene), respectively. In certain embodiments, the 5′ and 3′ homology arms are each 100% identical to the first and second genomic regions flanking the target locus (e.g., a target locus in the PAH gene), respectively. In certain embodiments, differences in nucleotide sequences of the 5′ homology arm and/or the 3′ homology arm and the corresponding regions the genome flanking a target locus comprise, consist essentially of, or consist of non-coding differences in nucleotide sequences.

The skilled worker will appreciate that homology arms do not need to be 100% identical to the genomic sequence flanking the target locus to be able to mediate integration of an editing element into that target locus by homologous recombination. For example, the homology arms can comprise one or more genetic variations in the human population, and/or one or more modifications (e.g., nucleotide substitutions, insertions, or deletions) designed to improve expression level or specificity. Human genetic variations include both inherited variations and de novo variations that are private to the target genome, and encompass simple nucleotide polymorphisms, insertions, deletions, rearrangements, inversions, duplications, micro-repeats, and combinations thereof. Such variations are known in the art, and can be found, for example, in the databases of dnSNP (see Sherry et al. Nucleic Acids Res. 2001; 29 (1): 308-11), the Database of Genomic Variants (see Nucleic Acids Res. 2014; 42 (Database issue): D986-92), ClinVar (see Nucleic Acids Res. 2014; 42 (Database issue): D980-D985), Genbank (see Nucleic Acids Res. 2016; 44 (Database issue): D67-D72), ENCODE (genome.ucsc.edu/encode/terms.html), JASPAR (see Nucleic Acids Res. 2018; 46 (D1): D260-D266), and PROMO (see Messeguer et al. Bioinformatics 2002; 18 (2): 333-334; Farré et al. Nucleic Acids Res. 2003; 31 (13): 3651-3653), each of which is incorporated herein by reference. The skilled worker will further appreciate that in situations where a homology arm is not 100% identical to the genomic sequence flanking the target locus, homologous recombination between the homology arm and the genome may alter the genomic sequence flanking the target locus such that it becomes identical to the sequence of the homology arm used.

In certain embodiments, the first genomic region 5′ to the target locus is located in a first editing window, wherein the first editing window consists of the region of the human genome corresponding to the nucleotide sequence set forth in SEQ ID NO: 37. In certain embodiments, the second genomic region 3′ to the target locus is located in a second editing window, wherein the second editing window consists of the region of the human genome corresponding to the nucleotide sequence set forth in SEQ ID NO: 38. In certain embodiments, the first genomic region 5′ to the target locus is located in a first editing window, wherein the first editing window consists of the region of the human genome corresponding to the nucleotide sequence set forth in SEQ ID NO: 37; and the second genomic region 3′ to the target locus is located in a second editing window, wherein the second editing window consists of the region of the human genome corresponding to the nucleotide sequence set forth in SEQ ID NO: 38.

In certain embodiments, the first and second editing windows are different. In certain embodiments, the first editing window is located 5′ to the second editing window. In certain embodiments, the first genomic region consists of a sequence shorter than the sequence of the first editing window in which the first genomic region is located. In certain embodiments, the first genomic region consists of the sequence of the first editing window in which the first genomic region is located. In certain embodiments, the second genomic region consists of a sequence shorter than the sequence of the second editing window in which the second genomic region is located. In certain embodiments, the second genomic region consists of the sequence of the second editing window in which the second genomic region is located.

In certain embodiments, the first and second editing windows are the same. In certain embodiments, the target locus is an internucleotide bond or a nucleotide sequence in the editing window, wherein the first genomic region consists of a first portion of the editing window 5′ to the target locus, and the second genomic region consists of a second portion of the editing window 3′ to the target locus. In certain embodiments, the first portion of the editing window consists of the sequence from the 5′ end of the editing window to the nucleotide adjacently 5′ to the target locus. In certain embodiments, the second portion of the editing window consists of the sequence from the nucleotide adjacently 3′ to the target locus to the 3′ end of the editing window. In certain embodiments, the first portion of the editing window consists of the sequence from the 5′ end of the editing window to the nucleotide adjacently 5′ to the target locus, and the second portion of the editing window consists of the sequence from the nucleotide adjacently 3′ to the target locus to the 3′ end of the editing window. In certain embodiments, the editing window consists of the region of the human genome corresponding to the nucleotide sequence set forth in SEQ ID NO: 37 or 38. In certain embodiments, the first and second portions of the editing windows have substantially equal lengths (e.g., the ratio of the length of the shorter portion to the length of the longer portion is greater than 0.5, 0.55, 0.6, 0.65, 0.7, 0.75, 0.8, 0.85, 0.9, 0.95, 0.96, 0.97, 0.98, or 0.99).

In certain embodiments, the 5′ homology arm has a length of about 50 to about 4000 nucleotides (e.g., about 100 to about 3000, about 200 to about 2000, about 500 to about 1000 nucleotides). In certain embodiments, the 5′ homology arm has a length of about 800 nucleotides. In certain embodiments, the 5′ homology arm has a length of about 100 nucleotides. In certain embodiments, the 3′ homology arm has a length of about 50 to about 4000 nucleotides (e.g., about 100 to about 3000, about 200 to about 2000, about 500 to about 1000 nucleotides). In certain embodiments, the 3′ homology arm has a length of about 800 nucleotides. In certain embodiments, the 3′ homology arm has a length of about 100 nucleotides. In certain embodiments, each of the 5′ and 3′ homology arms independently has a length of about 50 to about 4000 nucleotides (e.g., about 100 to about 3000, about 200 to about 2000, about 500 to about 1000 nucleotides). In certain embodiments, each of the 5′ and 3′ homology arms independently has a length of about 800 nucleotides.

In certain embodiments, the 5′ and 3′ homology arms have substantially equal nucleotide lengths. In certain embodiments, the 5′ and 3′ homology arms have asymmetrical nucleotide lengths. In certain embodiments, the asymmetry in nucleotide length is defined by a difference between the 5′ and 3′ homology arms of up to 90% in the length, such as up to an 80%, 70%, 60%, 50%, 40%, 30%, 20%, or 10% difference in the length.

In certain embodiments, the 5′ homology arm has at least about 90% (e.g., at least about 91%, 92%, 93%, 94%, 95%, 96%, 97%, 98%, 99%, or 99.5%) nucleotide sequence identity to the nucleotide sequence set forth in SEQ ID NO: 37. In certain embodiments, the 5′ homology arm further comprises one or more genetic variations in the human population. In certain embodiments, the 5′ homology arm comprises the nucleotide sequence set forth in SEQ ID NO: 23. In certain embodiments, the 5′ homology arm consists of the nucleotide sequence set forth in SEQ ID NO: 23.

In certain embodiments, the 3′ homology arm has at least about 90% (e.g., at least about 91%, 92%, 93%, 94%, 95%, 96%, 97%, 98%, 99%, or 99.5%) nucleotide sequence identity to the nucleotide sequence set forth in SEQ ID NO: 38. In certain embodiments, the 3′ homology arm further comprises one or more genetic variations in the human population. In certain embodiments, the 3′ homology arm comprises the nucleotide sequence set forth in SEQ ID NO: 24. In certain embodiments, the 3′ homology arm consists of the nucleotide sequence set forth in SEQ ID NO: 24.

In certain embodiments, the 5′ homology arm and the 3′ homology arm each has at least about 90% (e.g., at least about 91%, 92%, 93%, 94%, 95%, 96%, 97%, 98%, 99%, or 99.5%) nucleotide sequence identity to the nucleotide sequences set forth in SEQ ID NOs: 37 and 38, respectively. In certain embodiments, the 5′ homology arm and the 3′ homology arm each has at least about 90% (e.g., at least about 91%, 92%, 93%, 94%, 95%, 96%, 97%, 98%, 99%, or 99.5%) nucleotide sequence identity to the nucleotide sequences set forth in SEQ ID NOs: 23 and 24, respectively. In certain embodiments, the 5′ homology arm and the 3′ homology arm comprise the nucleotide sequences set forth in SEQ ID NOs: 37 and 38, 23 and 24, 37 and 24, or 23 and 38, respectively. In certain embodiments, the 5′ homology arm and the 3′ homology arm consist of the nucleotide sequences set forth in SEQ ID NOs: 37 and 38, 23 and 24, 37 and 24, or 23 and 38, respectively.

In certain embodiments, the rAAV genome comprises a nucleotide sequence at least 80% (e.g., at least 80%, 81%, 82%, 83%, 84%, 85%, 86%, 87%, 88%, 89%, 90%. 91% 92%, 93%, 94%, 95%, 96%, 97%, 98%, 99%, or 99.5%) identical to SEQ ID NO: 43. In certain embodiments, the rAAV genome comprises the nucleotide sequence set forth in SEQ ID NO: 43. In certain embodiments, the rAAV genome consists of the nucleotide sequence set forth in SEQ ID NO: 43.

In certain embodiments, the rAAV genomes disclosed herein further comprise a 5′ inverted terminal repeat (5′ ITR) nucleotide sequence 5′ of the TRE, and a 3′ inverted terminal repeat (3′ ITR) nucleotide sequence 3′ of the PAH coding sequence. ITR sequences from any AAV serotype or variant thereof can be used in the rAAV genomes disclosed herein. The 5′ and 3′ ITR can be from an AAV of the same serotype or from AAVs of different serotypes. Exemplary ITRs for use in the rAAV genomes disclosed herein are set forth in SEQ ID NOs: 14, 18, 19, 20, 21, and 32, herein.

In certain embodiments, the 5′ ITR or 3′ ITR is from AAV2. In certain embodiments, both the 5′ ITR and the 3′ ITR are from AAV2. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 14, or the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 18. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 14, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 18. In certain embodiments, the rAAV genome comprises a nucleotide sequence set forth in SEQ ID NO: 43, a 5′ ITR nucleotide sequence having the sequence of SEQ ID NO: 14, and a 3′ ITR nucleotide sequence having the sequence of SEQ ID NO: 18.

In certain embodiments, the 5′ ITR or 3′ ITR are from AAV5. In certain embodiments, both the 5′ ITR and 3′ ITR are from AAV5. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 20, or the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 21. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 20, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 21. In certain embodiments, the rAAV genome comprises a nucleotide sequence set forth in any one of SEQ ID NO: 43, a 5′ ITR nucleotide sequence having the sequence of SEQ ID NO: 20, and a 3′ ITR nucleotide sequence having the sequence of SEQ ID NO: 21.

In certain embodiments, the 5′ ITR nucleotide sequence and the 3′ ITR nucleotide sequence are substantially complementary to each other (e.g., are complementary to each other except for mismatch at 1, 2, 3, 4, or 5 nucleotide positions in the 5′ or 3′ ITR).

In certain embodiments, the 5′ITR or the 3′ ITR is modified to reduce or abolish resolution by Rep protein (“non-resolvable ITR”). In certain embodiments, the non-resolvable ITR comprises an insertion, deletion, or substitution in the nucleotide sequence of the terminal resolution site. Such modification allows formation of a self-complementary, double-stranded DNA genome of the AAV after the rAAV genome is replicated in an infected cell. Exemplary non-resolvable ITR sequences are known in the art (see e.g., those provided in U.S. Pat. Nos. 7,790,154 and 9,783,824, which are incorporated by reference herein in their entirety). In certain embodiments, the 5′ ITR comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19. In certain embodiments, the 5′ ITR consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19. In certain embodiments, the 5′ ITR consists of the nucleotide sequence set forth in SEQ ID NO: 19. In certain embodiments, the 3′ ITR comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 32. In certain embodiments, the 5′ ITR consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 32. In certain embodiments, the 3′ ITR consists of the nucleotide sequence set forth in SEQ ID NO: 32. In certain embodiments, the 5′ ITR consists of the nucleotide sequence set forth in SEQ ID NO: 19, and the 3′ ITR consists of the nucleotide sequence set forth in SEQ ID NO: 32. In certain embodiments, the 5′ITR consists of the nucleotide sequence set forth in SEQ ID NO: 19, and the 3′ ITR consists of the nucleotide sequence set forth in SEQ ID NO: 32.

In certain embodiments, the 5′ ITR is flanked by an additional nucleotide sequence derived from a wild-type AAV2 genomic sequence. In certain embodiments, the 5′ ITR is flanked by an additional 46 bp sequence derived from a wild-type AAV2 sequence that is adjacent to a wild-type AAV2 ITR in an AAV2 genome. In certain embodiments, the additional 46 bp sequence is 3′ to the 5′ ITR in the rAAV genome. In certain embodiments, the 46 bp sequence consists of the nucleotide sequence set forth in SEQ ID NO: 74.

In certain embodiments, the 3′ ITR is flanked by an additional nucleotide sequence derived from a wild-type AAV2 genomic sequence. In certain embodiments, the 3′ ITR is flanked by an additional 37 bp sequence derived from a wild-type AAV2 sequence that is adjacent to a wild-type AAV2 ITR in an AAV2 genome. See, e.g., Savy et al. Human Gene Therapy Methods (2017) 28 (5): 277-289 (which is hereby incorporated by reference herein in its entirety). In certain embodiments, the additional 37 bp sequence is 5′ to the 3′ ITR in the rAAV genome. In certain embodiments, the 37 bp sequence consists of the nucleotide sequence set forth in SEQ ID NO: 73.

In certain embodiments, the 5′ homology arm has at least about 80%, 81%, 82%, 83%, 84%, 85%, 86%, 87%, 88%, 89%, 90%, 91%, 92%, 93%, 94%, 95%, 96%, 97%, 98%, 99%, or 100% nucleotide sequence identity to the nucleotide sequence set forth in SEQ ID NO: 37. In certain embodiments, the 5′ homology arm further comprises one or more genetic variations in the human population. In certain embodiments, the 5′ homology arm comprises the nucleotide sequence set forth in SEQ ID NO: 23. In certain embodiments, the 5′ homology arm consists of the nucleotide sequence set forth in SEQ ID NO: 23.

In certain embodiments, the 3′ homology arm has at least about 80%, 81%, 82%, 83%, 84%, 85%, 86%, 87%, 88%, 89%, 90%, 91%, 92%, 93%, 94%, 95%, 96%, 97%, 98%, 99%, or 100% nucleotide sequence identity to the nucleotide sequence set forth in SEQ ID NO: 38. In certain embodiments, the 3′ homology arm further comprises one or more genetic variations in the human population. In certain embodiments, the 3′ homology arm comprises the nucleotide sequence set forth in SEQ ID NO: 24. In certain embodiments, the 3′ homology arm consists of the nucleotide sequence set forth in SEQ ID NO: 24.

In certain embodiments, the rAAV genome comprises from 5′ to 3′: a 5′ homology arm, a 5′ITR, a TRE, an intron element, at least a portion of a PAH coding sequence, a polyadenylation sequence, a 3′ ITR, and/or a 3′ homology arm. In certain embodiments, the 5′ homology arm has at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 37 or 23; the 5′ ITR has at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 14, 19, or 20; the TRE has at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 25, 26, 27, 30, 36, 39, 40, 41, 42, 44, 46, 47, 48, 49, 54, 55, 56, 57, 58, 59, 60, 62, 64, 65, 66, 67, 68, 69, 70, 72, or 72; the intron element has at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 29 or 61; the at least a portion of a PAH coding sequence has at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 28; the polyadenylation sequence has at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 31, 34, or 35; the 3′ ITR has at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 18, 21, or 32; and/or the 3′ homology arm has at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 38 or 24.

In certain embodiments, the 5′ homology arm comprises a nucleotide sequence selected from the group consisting of SEQ ID NOs: 37 and 23; the 5′ ITR comprises a nucleotide sequence selected from the group consisting of SEQ ID NOs: 14, 19, or 20; the TRE comprises a nucleotide sequence selected from the group consisting of SEQ ID NOs: 25-27, 30, 36, 39, 40-42, 44, 46-49, 54-60, and 62-72; the intron comprises a nucleotide sequence selected from the group consisting of SEQ ID NOs: 29 and 61; the at least a portion of a PAH coding sequence comprises the nucleotide sequence set forth in SEQ ID NO: 28; the polyadenylation sequence comprises a nucleotide sequence selected from the group consisting of SEQ ID NOs: 31, 34, and 35; the 3 ITR comprises a nucleotide sequence selected from the group consisting of SEQ ID NOs: 18, 21, and 32; and/or the 3′ homology arm comprises a nucleotide sequence selected from the group consisting of SEQ ID NOs: 38 and 24.

In certain embodiments, the 5′ homology arm comprises or consists of the nucleotide sequence set forth in SEQ ID NO: 23; the 5′ ITR comprises or consists of the nucleotide sequence set forth in SEQ ID NO: 14; the TRE comprises or consists of the nucleotide sequence set forth in SEQ ID NO: 25, 26, or 27; the intron element comprises or consists of the nucleotide sequence set forth in SEQ ID NO: 29; the at least a portion of a PAH coding sequence comprises or consists of the nucleotide sequence set forth in SEQ ID NO: 28; the polyadenylation sequence comprises or consists of the nucleotide sequence set forth in SEQ ID NO: 31; the 3′ ITR comprises or consists of the nucleotide sequence set forth in SEQ ID NO: 18; and/or the 5′ homology arm comprises or consists of the nucleotide sequence set forth in SEQ ID NO: 24.

In certain embodiments, the rAAV genome comprises from 5′ to 3′: the 5′ homology arm comprising the nucleotide sequence set forth in SEQ ID NO: 23; the 5′ ITR comprising the nucleotide sequence set forth in SEQ ID NO: 14; the TRE comprising from 5′ to 3′ the nucleotide sequence set forth in SEQ ID NO: 25, and the nucleotide sequence set forth in SEQ ID NO: 26 (e.g., the TRE comprises the nucleotide sequence set forth in SEQ ID NO: 27); the intron element comprising the nucleotide sequence set forth in SEQ ID NO: 29; the at least a portion of a PAH coding sequence comprising the nucleotide sequence set forth in SEQ ID NO: 28; the polyadenylation sequence comprising the nucleotide sequence set forth in SEQ ID NO: 31; the 3′ ITR comprising of the nucleotide sequence set forth in SEQ ID NO: 18; and the 5′ homology arm comprising the nucleotide sequence set forth in SEQ ID NO: 24.

In certain embodiments, the rAAV genome comprises a nucleotide sequence at least 80% (e.g., at least 80%, 81%, 82%, 83%, 84%, 85%, 86%, 87%, 88%, 89%, 90%. 91% 92%, 93%, 94%, 95%, 96%, 97%, 98%, or 99%) identical to SEQ ID NO: 45. In certain embodiments, the rAAV genome comprises the nucleotide sequence set forth in SEQ ID NO: 45. In certain embodiments, the rAAV genome consists of the nucleotide sequence set forth in SEQ ID NO: 45.

In another aspect, provided herein is a polynucleotide comprising a nucleic acid sequence that is at least 80% (e.g., at least 80%, 81%, 82%, 83%, 84%, 85%, 86%, 87%, 88%, 89%, 90%. 91% 92%, 93%, 94%, 95%, 96%, 97%, 98%, or 99%) identical to the nucleic acid sequence set forth in SEQ ID NO: 28, 43, 45, 51, or 52. In certain embodiments, the polynucleotide comprises or consists of the nucleic acid sequence set forth in SEQ ID NO: 43, 45, 51, or 52.

In another aspect, the instant disclosure provides pharmaceutical compositions comprising an AAV as disclosed herein together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current Ed., Mack Publishing Co., Easton Pa. 18042, USA; A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al, 7th ed., Lippincott, Williams, & Wilkins; and Handbook of Pharmaceutical Excipients (2000) A. H. Kibbe et al, 3rd ed. Amer. Pharmaceutical Assoc.

In another aspect, the instant disclosure provides a polynucleotide comprising a coding sequence encoding a human PAH protein or a fragment thereof, wherein the coding sequence has been silently altered to have less than 100% (e.g., less than 99%, 98%, 97%, 96%, 95%, 90%, 85%, 80%, 75%, 70%, 65%, 60%, 55%, or 50%) identical to a wild-type human PAH gene. In certain embodiments, the polynucleotide comprises the nucleotide sequence set forth in SEQ ID NO: 28. The polynucleotide can comprise DNA, RNA, modified DNA, modified RNA, or a combination thereof. In certain embodiments, the polynucleotide is an expression vector.

The AAV compositions disclosed herein are particularly advantageous in that they are capable of editing a PAH gene in a cell with high efficiency both in vivo and in vitro. In certain embodiments, the integration efficiency of the editing element into the target locus is at least 1% (e.g., at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%) when the AAV is administered to a mouse implanted with human hepatocytes in the absence of an exogenous nuclease under standard AAV administration conditions. In certain embodiments, the allelic frequency of integration of the editing element into the target locus is at least 0.5% (e.g., at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%) when the AAV is administered to a mouse implanted with human hepatocytes in the absence of an exogenous nuclease under standard AAV administration conditions.

Any methods of determining the efficiency of editing of the PAH gene can be employed. In certain embodiments, individual cells are separated from the population of transduced cells and subject to single-cell PCR using PCR primers that can identify the presence of an editing element correctly integrated into the target locus of the PAH gene. Such method can further comprise single-cell PCR of the same cells using PCR primers that selectively amplify an unmodified target locus. In this way, the genotype of the cells can be determined. For example, if the single cell PCR showed that a cell has both an edited target locus and an unmodified target locus, then the cell would be considered heterozygous for the edited PAH gene.

Additionally or alternatively, in certain embodiments, linear amplification mediated PCR (LAM-PCR), quantitative PCR (qPCR) or digital droplet PCR (ddPCR) can be performed on DNA extracted from the population of transduced cells using primers and probes that only detect edited PAH alleles. Such methods can further comprise an additional qPCR or ddPCR (either in the same reaction or a separate reaction) to determine the number of total genomes in the sample and the number of unedited PAH alleles. These numbers can be used to determine the allelic frequency of integration of the editing element into the target locus.

Additionally or alternatively, in certain embodiments, the PAH locus can be amplified from DNA extracted from the population of transduced cells either by PCR using primers that bind to regions of the PAH gene flanking the target locus, or by LAM-PCR using a primer that binds a region within the rAAV genome (e.g., a region comprising an exogenous sequence non-native to the locus). The resultant PCR amplicons can be individually sequenced using single molecule next generation sequencing (NGS) techniques to determine the relative number of edited and unedited PAH alleles present in the population of transduced cells. These numbers can be used to determine the allelic frequency of integration of the editing element into the target locus.

In another aspect, the instant disclosure provides pharmaceutical compositions comprising an AAV as disclosed herein together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current Ed., Mack Publishing Co., Easton Pa. 18042, USA; A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al, 7th ed., Lippincott, Williams, & Wilkins; and Handbook of Pharmaceutical Excipients (2000) A. H. Kibbe et al, 3rd ed. Amer. Pharmaceutical Assoc.

### III. METHODS OF USE

In another aspect, the instant disclosure provides methods for restoring PAH gene function in a cell. The methods generally comprise transducing the cell with an rAAV as disclosed herein. Such methods are highly efficient at editing the PAH gene function in a cell, and do not require cleavage of the genome at the target locus by the action of an exogenous nuclease (e.g., a meganuclease, a zinc finger nuclease, a transcriptional activator-like nuclease (TALEN), or an RNA-guided nuclease such as a Cas9) to facilitate such correction. Accordingly, in certain embodiments, the methods disclosed herein involve transducing the cell with an rAAV as disclosed herein without co-transducing or co-administering an exogenous nuclease or a nucleotide sequence that encodes an exogenous nuclease.

The methods disclosed herein can be applied to any cell harboring a mutation in the PAH gene. The skilled worker will appreciate that cells that actively express PAH are of particular interest. Accordingly, in certain embodiments, the method is applied to cells in the liver, kidney, brain, pituitary gland, adrenal gland, pancreas, urinary bladder, gallbladder, colon, small intestine, or breast. In certain embodiments, the method is applied to hepatocytes and/or renal cells.

The methods disclosed herein can be performed in vitro for research purposes or can be performed ex vivo or in vivo for therapeutic purposes.

In certain embodiments, the cell to be transduced is in a mammalian subject and the AAV is administered to the subject in an amount effective to transduce the cell in the subject. Accordingly, in certain embodiments, the instant disclosure provides a method for treating a subject having a disease or disorder associated with a PAH gene mutation, the method generally comprising administering to the subject an effective amount of an rAAV as disclosed herein. The subject can be a human subject or a rodent subject (e.g., a mouse) containing human liver cells. Suitable mouse subjects include without limitation, mice into which human liver cells (e.g., human hepatocytes) have been engrafted. Any disease or disorder associated with a PAH gene mutation can be treated using the methods disclosed herein. Suitable diseases or disorders include, without limitation, phenylketonuria. In certain embodiments, the cell is transduced without co-transducing or co-administering an exogenous nuclease or a nucleotide sequence that encodes an exogenous nuclease.

The methods disclosed herein are particularly advantageous in that they are capable of editing a PAH gene in a cell with high efficiency both in vivo and in vitro. In certain embodiments, the integration efficiency of the editing element into the target locus is at least 1% (e.g., at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%) when the AAV is administered to a mouse implanted with human hepatocytes in the absence of an exogenous nuclease under standard AAV administration conditions. In certain embodiments, the allelic frequency of integration of the editing element into the target locus is at least 0.5% (e.g., at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%) when the AAV is administered to a mouse implanted with human hepatocytes in the absence of an exogenous nuclease under standard AAV administration conditions.

The methods disclosed herein can be performed in vitro for research purposes or can be performed ex vivo or in vivo for therapeutic purposes.

In certain embodiments, the cell to be transduced is in a mammalian subject and the AAV is administered to the subject in an amount effective to transduce the cell in the subject. Accordingly, in certain embodiments, the instant disclosure provides a method for treating a subject having a disease or disorder associated with a PAH gene mutation, the method generally comprising administering to the subject an effective amount of an rAAV as disclosed herein. The subject can be a human subject, a non-human primate subject (e.g., a cynomolgus), or a rodent subject (e.g., a mouse) with a PAH gene mutation. Any disease or disorder associated with a PAH gene mutation can be treated using the methods disclosed herein. Suitable diseases or disorders include, without limitation, phenylketonuria (PKU).

In certain embodiments, the foregoing methods employ an rAAV comprising: (a) an AAV capsid protein comprising the amino acid sequence of amino acids 203-736 of SEQ ID NO: 16, and an rAAV genome comprising 5′ to 3′ following genetic elements: a 5′ homology arm (e.g., the 5′ homology arm comprising the nucleotide sequence set forth in SEQ ID NO: 23), a 5′ ITR (e.g., the 5′ ITR comprising the nucleotide sequence set forth in SEQ ID NO: 14), a transcriptional regulatory element (e.g., a TRE comprising the nucleotide sequence set forth in SEQ ID NO: 27), an intron element (e.g., the intron element comprising the nucleotide sequence set forth in SEQ ID NO: 29), at least a portion of a PAH coding sequence (e.g., the PAH coding sequence comprising the nucleotide sequence set forth in SEQ ID NO: 28), a polyadenylation sequence (e.g., the SV40 polyadenylation sequence of SEQ ID NO: 31), a 3′ ITR (e.g., the 3′ ITR comprising the nucleotide sequence set forth in SEQ ID NO: 18), and a 3′ homology arm (e.g., the 3′ homology arm comprising the nucleotide sequence set forth in SEQ ID NO: 24); (b) an AAV capsid protein comprising the amino acid sequence of amino acids 138-736 of SEQ ID NO: 16, and an rAAV genome comprising 5′ to 3′ following genetic elements: a 5′ homology arm (e.g., the 5′ homology arm comprising the nucleotide sequence set forth in SEQ ID NO: 23), a 5′ ITR (e.g., the 5′ ITR comprising the nucleotide sequence set forth in SEQ ID NO: 14), a transcriptional regulatory element (e.g., a TRE comprising the nucleotide sequence set forth in SEQ ID NO: 27), an intron element (e.g., the intron element comprising the nucleotide sequence set forth in SEQ ID NO: 29), at least a portion of a PAH coding sequence (e.g., the PAH coding sequence comprising the nucleotide sequence set forth in SEQ ID NO: 28), a polyadenylation sequence (e.g., the SV40 polyadenylation sequence of SEQ ID NO: 31), a 3′ ITR (e.g., the 3′ ITR comprising the nucleotide sequence set forth in SEQ ID NO: 18), and a 3′ homology arm (e.g., the 3′ homology arm comprising the nucleotide sequence set forth in SEQ ID NO: 24); and/or (c) an AAV capsid protein comprising the amino acid sequence of SEQ ID NO: 16, and an rAAV genome comprising 5′ to 3′ following genetic elements: a 5′ homology arm (e.g., the 5′ homology arm comprising the nucleotide sequence set forth in SEQ ID NO: 23), a 5′ITR (e.g., the 5′ ITR comprising the nucleotide sequence set forth in SEQ ID NO: 14), a transcriptional regulatory element (e.g., a TRE comprising the nucleotide sequence set forth in SEQ ID NO: 27), an intron element (e.g., the intron element comprising the nucleotide sequence set forth in SEQ ID NO: 29), at least a portion of a PAH coding sequence (e.g., the PAH coding sequence comprising the nucleotide sequence set forth in SEQ ID NO: 28), a polyadenylation sequence (e.g., the SV40 polyadenylation sequence of SEQ ID NO: 31), a 3′ ITR (e.g., the 3′ ITR comprising the nucleotide sequence set forth in SEQ ID NO: 18), and a 3′ homology arm (e.g., the 3′ homology arm comprising the nucleotide sequence set forth in SEQ ID NO: 24).

In certain embodiments, transduction of a cell with an AAV composition disclosed herein can be performed as provided herein or by any method of transduction known to one of ordinary skill in the art. In certain embodiments, the cell may be contacted with the AAV at a multiplicity of infection (MOI) of 50,000; 100,000; 150,000; 200,000; 250,000; 300,000; 350,000; 400,000; 450,000; or 500,000, or at any MOI that provides for optimal transduction of the cell.

An AAV composition disclosed herein can be administered to a subject by any appropriate route including, without limitation, intravenous, intraperitoneal, subcutaneous, intramuscular, intranasal, topical or intradermal routes. In certain embodiments, the composition is formulated for administration via intravenous injection or subcutaneous injection.

### IV. AAV PACKAGING SYSTEMS

In another aspect, the instant disclosure provides packaging systems for recombinant preparation of a recombinant adeno-associated virus (rAAV) disclosed herein. Such packaging systems generally comprise: first nucleotide encoding one or more AAV Rep proteins; a second nucleotide encoding a capsid protein of any of the AAVs as disclosed herein; and a third nucleotide sequence comprising any of the rAAV genomes as disclosed herein, wherein the packaging system is operative in a cell for enclosing the rAAV genome in the capsid to form the AAV.

In certain embodiments, the packaging system comprises a first vector comprising the first nucleotide sequence encoding the one or more AAV Rep proteins and the second nucleotide sequence encoding the AAV capsid protein, and a second vector comprising the third nucleotide sequence comprising the rAAV genome. As used in the context of a packaging system as described herein, a “vector” refers to a nucleic acid molecule that is a vehicle for introducing nucleic acids into a cell (e.g., a plasmid, a virus, a cosmid, an artificial chromosome, etc.).

Any AAV Rep protein can be employed in the packaging systems disclosed herein. In certain embodiments of the packaging system, the Rep nucleotide sequence encodes an AAV2 Rep protein. Suitable AAV2 Rep proteins include, without limitation, Rep 78/68 or Rep 68/52. In certain embodiments of the packaging system, the nucleotide sequence encoding the AAV2 Rep protein comprises a nucleotide sequence that encodes a protein having a minimum percent sequence identity to the AAV2 Rep amino acid sequence of SEQ ID NO: 22, wherein the minimum percent sequence identity is at least 70% (e.g., at least 75%, at least 80%, at least 85%, at least 90%, at least 95%, at least 98%, at least 99%, or 100%) across the length of the amino acid sequence of the AAV2 Rep protein. In certain embodiments of the packaging system, the AAV2 Rep protein has the amino acid sequence set forth in SEQ ID NO: 22.

In certain embodiments of the packaging system, the packaging system further comprises a fourth nucleotide sequence comprising one or more helper virus genes. In certain embodiments of the packaging system, the packaging system further comprises a third vector, e.g., a helper virus vector, comprising the fourth nucleotide sequence comprising the one or more helper virus genes. The third vector may be an independent third vector, integral with the first vector, or integral with the second vector.

In certain embodiments of the packaging system, the helper virus is selected from the group consisting of adenovirus, herpes virus (including herpes simplex virus (HSV)), poxvirus (such as vaccinia virus), cytomegalovirus (CMV), and baculovirus. In certain embodiments of the packaging system, where the helper virus is adenovirus, the adenovirus genome comprises one or more adenovirus RNA genes selected from the group consisting of E1, E2, E4 and VA. In certain embodiments of the packaging system, where the helper virus is HSV, the HSV genome comprises one or more of HSV genes selected from the group consisting of UL5/8/52, ICPO, ICP4, ICP22 and UL30/UL42.

In certain embodiments of the packaging system, the first, second, and/or third vector are contained within one or more plasmids. In certain embodiments, the first vector and the third vector are contained within a first plasmid. In certain embodiments the second vector and the third vector are contained within a second plasmid.

In certain embodiments of the packaging system, the first, second, and/or third vector are contained within one or more recombinant helper viruses. In certain embodiments, the first vector and the third vector are contained within a recombinant helper virus. In certain embodiments, the second vector and the third vector are contained within a recombinant helper virus.

In a further aspect, the disclosure provides a method for recombinant preparation of an AAV as described herein, wherein the method comprises transfecting or transducing a cell with a packaging system as described herein under conditions operative for enclosing the rAAV genome in the capsid to form the rAAV as described herein. Exemplary methods for recombinant preparation of an rAAV include transient transfection (e.g., with one or more transfection plasmids containing a first, and a second, and optionally a third vector as described herein), viral infection (e.g., with one or more recombinant helper viruses, such as a adenovirus, poxvirus (such as vaccinia virus), herpes virus (including HSV, cytomegalovirus, or baculovirus), containing a first, and a second, and optionally a third vector as described herein), and stable producer cell line transfection or infection (e.g., with a stable producer cell, such as a mammalian or insect cell, containing a Rep nucleotide sequence encoding one or more AAV Rep proteins and/or a Cap nucleotide sequence encoding one or more AAV capsid proteins as described herein, and with an rAAV genome as described herein being delivered in the form of a plasmid or a recombinant helper virus).

Accordingly, the instant disclosure provides a packaging system for preparation of a recombinant AAV (rAAV), wherein the packaging system comprises a first nucleotide sequence encoding one or more AAV Rep proteins; a second nucleotide sequence encoding a capsid protein of any one of the AAVs described herein; a third nucleotide sequence comprising an rAAV genome sequence of any one of the AAVs described herein; and optionally a fourth nucleotide sequence comprising one or more helper virus genes.

### V. EXAMPLES

These examples are offered by way of illustration, and not by way of limitation.

### Example 1: hPAH Correction Vector Designs

### Mouse-Specific PAH Gene Editing Vector PAH-006m

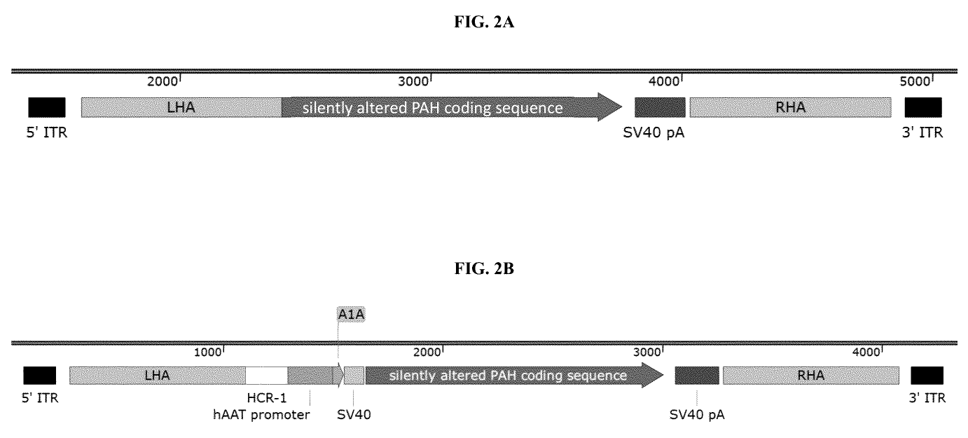

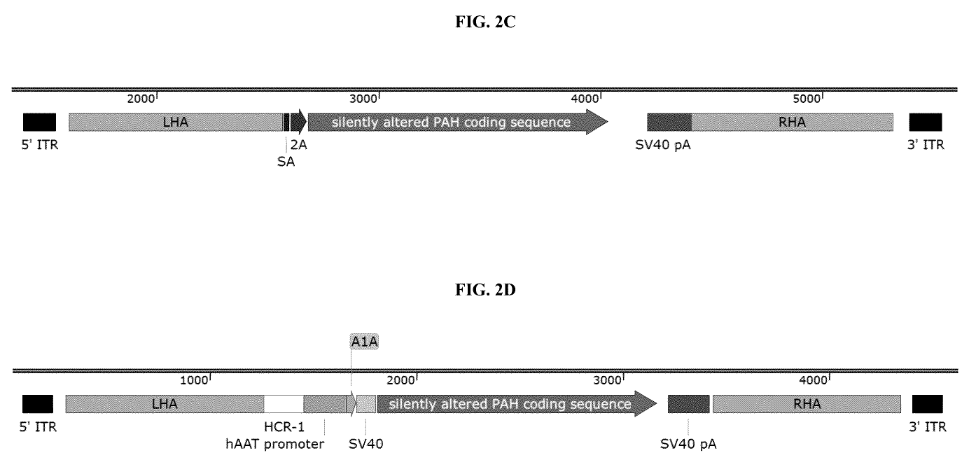

The mouse-specific gene editing AAV vector, PAH-006m, is shown in FIG. 2A. This vector was designed to integrate a human PAH coding sequence into the mouse PAH gene locus using left and right homology arms that are specific to the mouse sequence. PAH-006m comprises, from 5′ to 3′, the following genetic elements: a 5′ITR element; a 5′ homology arm; a silently altered human PAH coding sequence; an SV40 polyadenylation sequence; a 3′ homology arm; and a 3′ ITR element. The sequences of these elements are set forth in Table 1. PAH-006m does not comprise a heterologous promoter.

### Mouse-Specific PAH Gene Transfer/Gene Editing Vector PAH-006m-LP-1

The mouse-specific gene transfer/gene editing AAV vector, PAH-006m-LP-1, is shown in FIG. 2B. This vector was designed to integrate a human PAH coding sequence into the mouse PAH gene locus using mouse left and right homology arms, and also to allow expression of human PAH under the control of a heterologous liver-specific promoter in a cell in the absence of genomic integration. PAH-006m-LP-1 comprises, from 5′ to 3′, the following genetic elements: a 5′ ITR element; a 5′ homology arm; a transcriptional regulatory element comprising a human apolipoprotein hepatic control region (HCR) element, a human alpha-1-antitrypsin (hAAT) promoter element, and a hAAT exon 1; an SV40 element; a silently altered human PAH coding sequence; an SV40 polyadenylation sequence; a 3′ homology arm; and a 3′ ITR element. The sequences of these elements are set forth in Table 1.

### Human-Specific PAH Gene Editing Vector PAH-032h

The human-specific gene editing AAV vector, PAH-032h, is shown in FIG. 2C. This vector was designed to integrate a human PAH coding sequence into the human PAH gene locus using left and right homology arms that are specific to the human sequence. PAH-032h comprises, from 5′ to 3′, the following genetic elements: a 5′ ITR element; a 5′ homology arm; a splice acceptor; a 2A element; a silently altered human PAH coding sequence; an SV40 polyadenylation sequence; a 3′ homology arm; and a 3′ ITR element. The sequences of these elements are set forth in Table 1. PAH-032h does not comprise a heterologous promoter.

Human-Specific PAH Gene Transfer/Gene Editing Vector hPAH-hI1C-032-LP1-SD3

The human-specific gene transfer/gene editing AAV vector, hPAH-hI1C-032-LP1-SD3, is shown in FIG. 2D. This vector was designed to integrate a human PAH coding sequence into the human PAH gene locus using human left and right homology arms, and also to allow expression of human PAH under the control of a heterologous liver-specific promoter in a cell in the absence of genomic integration. hPAH-hI1C-032-LP1-SD3 comprises, from 5′ to 3′, the following genetic elements: a 5′ ITR element; a 5′ homology arm; a transcriptional regulatory element comprising a human apolipoprotein hepatic control region (HCR) element, a human alpha-1-antitrypsin (hAAT) promoter element, and a hAAT exon 1; an SV40 element; a silently altered human PAH coding sequence; an SV40 polyadenylation sequence; a 3′ homology arm; and a 3′ ITR element. The sequences of these elements are set forth in Table 1.

TABLE 1
Selected genetic elements in PAH correction vectors PAH-006m,
PAH-006m-LP-1, PAH-032h, and hPAH-hI1C-032-LP1-5D3

| | PAH- | PAH-006m- | PAH- | hPAH-hI1C- |
|---|---|---|---|---|
| | 006m | LP-1 | 032h | 032-LP1-5D3 |
| Genetic Element | SEQ ID NO: | | | |
| 5′ ITR element | 14 | 14 | 14 | 14 |
| 5′ homology arm | 84 | 85 | 23 | 23 |
| Splice acceptor | — | — | 30 | — |
| 2A element | — | — | 40 | — |
| transcriptional regulatory | — | 27 | — | 27 |
| element | | | | |
| SV40 intron element | — | 29 | — | 29 |
| silently altered human PAH | 83 | 28 | 83 | 28 |
| coding sequence | | | | |
| SV40 polyadenylation | 31 | 31 | 31 | 31 |
| sequence | | | | |
| 3′ homology arm | 86 | 86 | 24 | 24 |
| 3′ ITR element | 18 | 18 | 18 | 18 |
| AAV genome from 5′ | 77 | 79 | 81 | 43 |
| homology arm to 3′ | | | | |
| homology arm | | | | |
| AAV genome from 5′ ITR | 78 | 80 | 82 | 45 |
| to 3′ ITR | | | | |

“—” signifies an absence of this element

### Example 2: Analysis of hPAH Correction Vectors in Mouse Models

### Materials and Methods

### PAH^{enu2} Mouse Model:

The PAH^{enu2} mouse model was used to establish dose response for PAH correction vectors packaged in AAVHSC15 capsid through measurement of vector genomes, percent target gene insertion, hPAH mRNA expression, and Phe/Tyr concentration.

The PAH^{enu2} mouse line was produced by germline ethylnitrosourea mutagenesis followed by a Phe clearance screen to isolate a mutant mouse line deficient in PAH activity (see McDonald J D, et al. PNAS 1990; 87:1965-1967, which is hereby incorporated by reference in its entirety). It has a homozygous missense mutation (F263S in exon 7) in the region encoding the PAH active site, resulting in abolished activity of PAH. Mutations within exon 7 are common in humans with PAH deficiency, although PAH deficiency in humans is typically expressed in a compound heterozygous background. PAH^{enu2} mice exhibit present with blood Phe levels consistently above 1200 μmol/L.

### FRG® Mouse Human Liver Xenograft Model:

Fah^{-/-}/Rag2^{-/-}/Il2rg^{-/-} (FRG) mice (Azuma et al. (2007) Nat. Biotechnol. 25 (8): 903-910) containing human liver tissue were used to measure in vivo transduction of vector genomes, human DNA target gene insertion, and mRNA expression of hPAH in human liver tissue, following administration of correction vectors packaged in AAVHSC15 capsid across a range of doses.

The FRG® human liver xenograft model is an immunocompromised mouse strain harboring triple gene knockouts of Fah^{-/-}, IL2rg^{-/-} and Rag2^{-/-}. Because of the IL2rg^{-/-} and Rag2^{-/-} knockouts these mice lack B-cell, T-cell and NK-cells resulting in acceptance of engraftment with human cells. The Fah^{-/-} genotype renders these mice dependent on the liver protective drug, 2-(2-nitro-4-trifluoromethylbenzoyl)-1,3-cyclohexanedione (NTBC), such that its withdrawal leads to loss of hepatocytes and animal death within 4-8 weeks.

To generate humanized livers in FRG mice, human primary hepatocytes were implanted concurrent with NTBC withdrawal. Since the human hepatocytes have an intact FAH gene, only murine hepatocytes are affected, resulting in the gradual repopulation of the mouse liver compartment with human hepatocytes. The human repopulated livers show >90% human hepatocytes and restored hepatic function (Azuma et al. (2007) Nat. Biotechnol. 25 (8): 903-910).

On-Target Insertion—ddPCR Linkage Assay:

To determine the level of target insertion per allele at the PAH gene locus, an assay that uses droplet digital PCR (ddPCR) was employed. In this assay, target insertion was determined by measuring genetic linkage between the hPAH transgene and the genomic target. Linkage is a measurement of how often two sequences are on the same strand of DNA. Determining the amount of linked to unlinked vector and genome sequences can be used to measure insertion efficiency, which is reported as percent insertion per allele.

To measure target insertion via linkage, two primer and probe sets were used, one targeting the silently altered hPAH payload and a second targeting the Pah genomic sequence beyond the homology arms. Each probe consisted of a fluorophore in either the FAM or HEX channel such that when DNA is analyzed in partitioned droplets, each droplet can be scored by its emission signature as producing one of three possible signals: FAM-positive (silently altered hPAH payload alone); HEX-positive (genomic alone); and HEX+FAM positive (contains silently altered hPAH payload and genomic sequence). Target insertion was measured by determining the proportion of droplets containing both the payload and the genomic sequence in excess of expectation due to probability, divided by the total number of Pah alleles tested.

As the relative concentration of vector to genome can vary across doses and models, it is important to ensure each measurement is within the linear range of detection for both the vector and the target genomic sequence. Therefore, prior to measuring target insertion, the relative abundance of vector and target genome was measured across a range of sample input concentrations from 1 ng, 5 ng, 10 ng, 100 ng. The ratio of vector to genome was used to determine the sample input concentration in which both the vector and genomes fall within the limits of detection by ddPCR which are between 0.25-5000 molecules per μl.

### On-Target Insertion—Next Generation Sequencing (NGS) Assay:

This approach was used to calculate target insertion using assays spanning both left and right integration sites of the human PAH editing construct. Each of the two amplicons share their respective outward primer located on the genomic DNA flanking each homology arm, while the inward primers were unique for unedited alleles or for the edited alleles respectively. Each number of wild-type (WT) and edited sequences were tallied by counting the number of sequences covering the junction between the homology arm and either the inserted gene or the unedited wild-type sequence. Having separate assays covering both the left and right side of the insertion site provides redundant quantitation of insertion. By using this NGS approach, the percentage of target insertions can be detected per total number of alleles, and in addition, sequences of the entire insertion site can be collected, enabling the detection of de novo mutations (e.g., incorrect insertion and deletion events and ITR integration).

Differences of uninserted and inserted amplification efficiency were accounted for using an 11-step standard for both left and right integration sites. Each control panel consists of purified uninserted amplicon and edited amplicon at the following ratios: 0% edited, 1%, 2%, 5%, 10% 50%, 60%, 90%, 95%, 98%, 99% and 100% edited control sequence. Target insertion efficiency calculations were as follows: the sequencing reads specific to uninserted and inserted genomic loci are tallied and target insertion was calculated by (insertion read counts/total read counts*100%) and fitted to the standard curve. As editing quantitation per sample was measured by two independent assays, each spanning opposite homology arms into the native genome, the consistency between each measurement was determined.

### On-Target Accuracy—Next Generation Sequencing (NGS) Assay:

To determine if target gene insertion is accompanied by de novo mutation at the integration sites, PCR amplicons spanning genomic sequences beyond both left and right of the homologous arms and into the target insertion site were sequenced. A total of four amplicons were sequenced per sample. Insertion specific amplicons spanning each homology arm (hPAH dependent PCR) or WT/uninserted alleles spanning each homology arm (no hPAH insertion) were assessed.

To identify the detection limit of the assay, an amplicon-based control panel was built by mixing two right homologous arm amplicons, varied by one base (a T to G variation), at 0%, 0.1%, 0.5%, 1%, 2%, 5%, 10%, 50, and 100%. The amplicon control panel went through the same processing steps. Based on the quantitative variant calling from the standard curve, false-positive variants were observed at a frequency of 0.1-0.25% at non-random positions. Sequencing coverage for almost all positions was above 10,000. A variant was considered to be a true positive with the following criteria: (1) at >0.5% variant frequency and pass filters; (2) appear in both technical sequencing duplicates; and (3) not observed in WT alleles given comparable sequence depth and quality.

### Results

A study was performed in PAH^{enu2} mice using the mouse-specific correction vector, PAH-006m, which contains homology arms targeting the mouse PAH locus. PAH-006m does not contain a liver specific promoter, and as such, expression is driven by the endogenous promoter elements. Three male mice per group were administered either 1E+14 vg/kg PAH-006m packaged in AAVHSC15 capsid, or formulation buffer, and blood Phe level was measured weekly for 12 weeks. At the 12-week timepoint, mice were sacrificed to measure target insertion at the PAH locus, and mRNA expression levels.

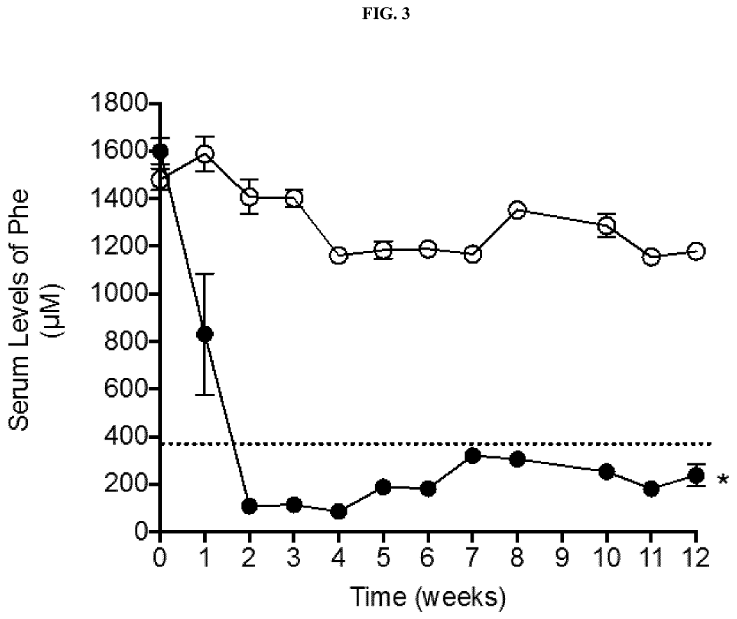

Blood Phe levels were reduced to normal levels by 2 weeks post-administration of PAH-006m packaged in AAVHSC15 capsid, as shown in FIG. 3. Reduction of blood Phe was maintained at levels of <360 μM for the duration of the 12-week study.

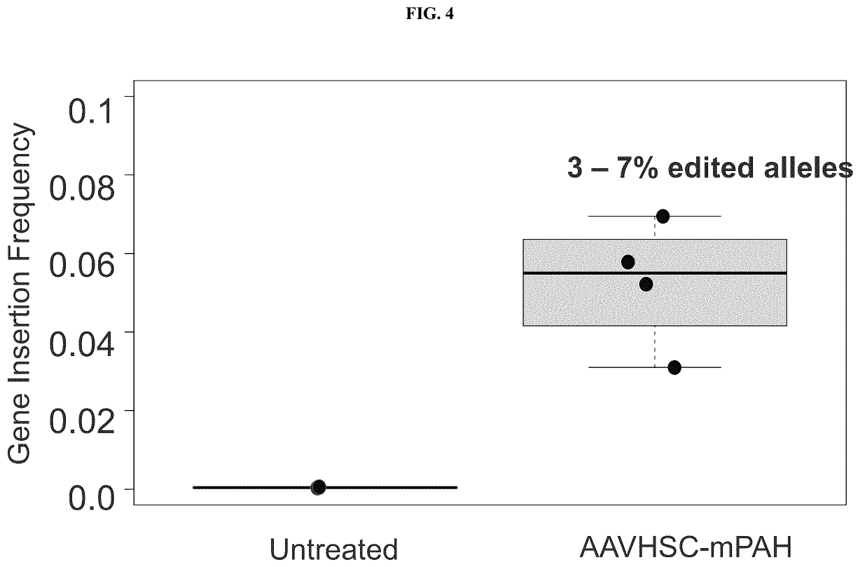

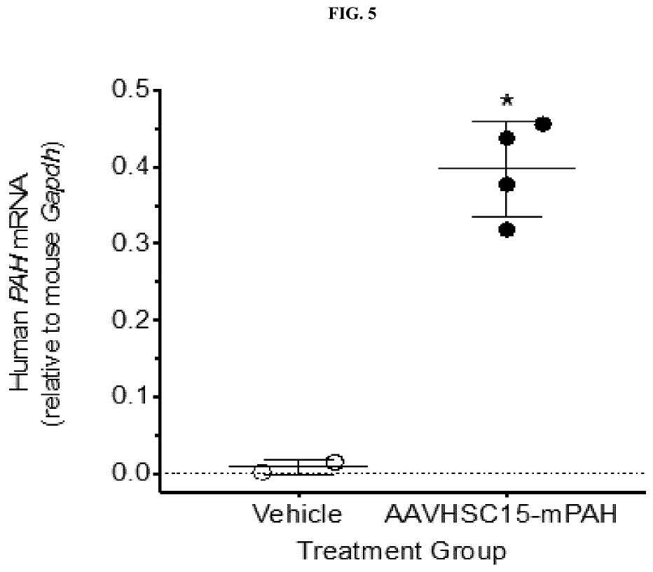

The level of on-target insertion at the PAH locus was measured using the ddPCR linkage assay as described in the Materials and Methods above. A range of 3-7% target insertion per allele was detected in the liver at the 12-week timepoint, as shown in FIG. 4. The level of silently altered hPAH mRNA was also measured (relative to mouse GAPDH) and is shown in FIG. 5. The expression of silently altered hPAH mRNA was detected (PAH/GAPDH mRNA ratio was 0.38±0.06). Of note, the level of on-target insertion per allele was found to be directly correlated to the level of mRNA expression (R-squared 0.82; P<0.02).

To determine if similar levels of on-target insertion and mRNA expression could be achieved in human liver, experiments were conducted in the FRG® mouse containing a human liver xenograft. Mice were treated with a single IV dose of 1E+14 vg/kg PAH-032h packaged in AAVHSC15 capsid. PAH-032h does not contain the liver specific promoter, and as such, expression is driven by the endogenous human promoter elements, similar to construct PAH-006m.

Expression of hPAH mRNA derived from silently altered hPAH was evaluated in liver cells from the FRG® mouse. Expression was measured by RT-ddPCR and expressed as a ratio to GAPDH to allow for comparison across human and mouse hepatocytes. In the human hepatocytes isolated from the FRG® mouse model, 6-weeks after administration of PAH-032h packaged in AAVHSC15 capsid, hPAH mRNA derived from silently altered hPAH was expressed (hPAH/GAPDH mRNA ratio=0.57±0.11). Levels of silently altered hPAH mRNA in mouse hepatocytes were significantly lower (PAH/GAPDH mRNA ratio=0.13±0.08, p<0.003 vs human) and could be entirely accounted for by the presence of contaminating human hepatocytes in the preparations used (approximately 15-20% human). The proportion of mouse cells in human samples and human cells in mouse samples was calculated by measuring the relative abundance of human and mouse GAPDH gene in each sample.

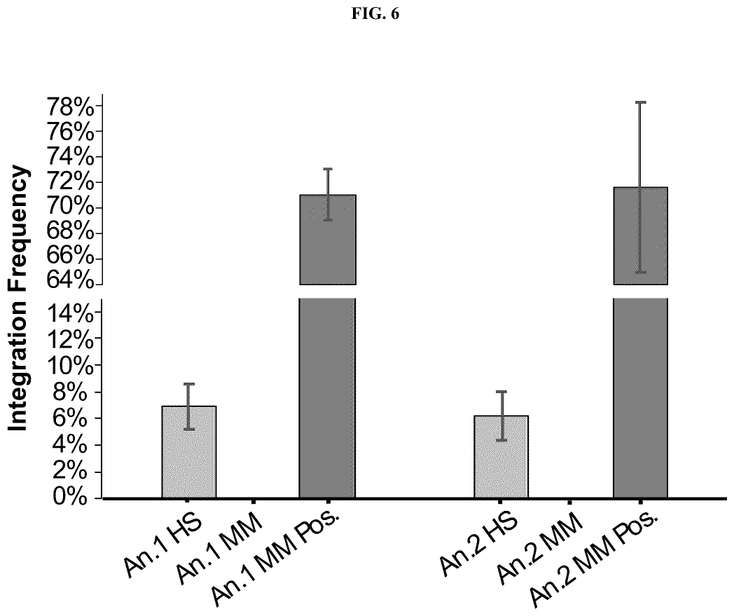

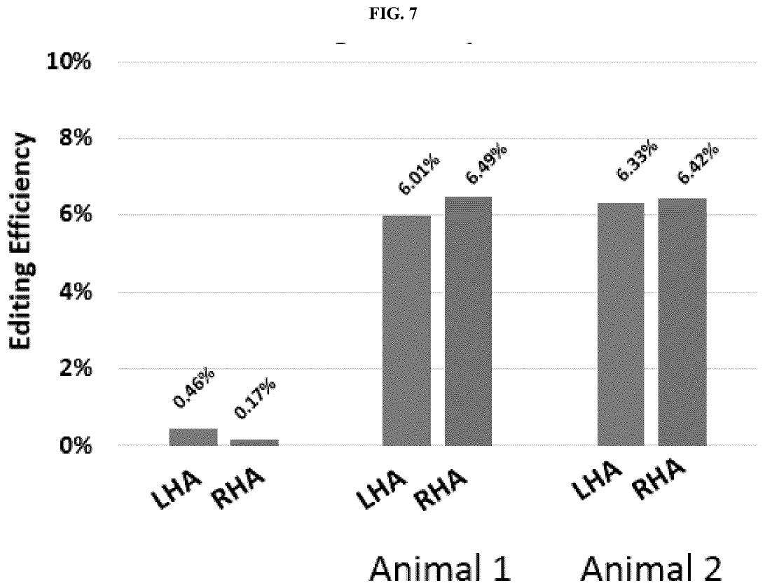

Confirmation of human-specific target insertion into the human PAH locus was done in a separate study in FRGR mice where levels of on-target insertion (˜6% using the linkage ddPCR method; FIG. 6) and levels of hPAH mRNA derived from silently altered hPAH (hPAH/GAPDH mRNA ratio=0.45±0.26) were achieved in human cells with treatment of PAH-032h packaged in AAVHSC15 capsid. Target gene insertion efficiency was characterized using the NGS assay in which quantitation of integration is based on two separate measurements that cover both the left and right homology arms, respectively, with coverage extending into the genomic targeted integration sites and past the end of each homology arm, as described in the Materials and Methods above. Untreated wild-type genomic DNA was used to detect the false-positive rate for this experiment. Integration of hPAH into the PAH locus was confirmed with orthogonal assays (linkage ddPCR and NGS) reporting integration efficiencies of 6%. These results are shown in FIG. 7.

FIG. 6 and FIG. 7 show targeted insertion frequency in hepatocytes isolated from treated FRG mice. FIG. 6 shows insertion of the human-targeting PAH-032h correction vector packaged in AAVHSC15 capsid into mouse and human PAH loci, as measured by the species specific ddPCR editing assays described herein. As a positive control linkage between a mouse-specific PAH locus probe and a probe for the homologous insertion site was used. In FIG. 6, An. 1 HS refers to Animal 1 Homo sapiens Cells, An. 1 MM refers to Animal 1 Mus musculus cells, An.1 MM Pos. refers to Animal 1 Mus musculus positive control, An.2 HS refers to Animal 2 Homo sapiens Cells, An.2 MM refers to Animal 2 Mus musculus cells, and An.2 MM Pos. refers to Animal 2 Mus musculus positive control. FIG. 7 shows the editing of human hepatocytes as measured by quantitative NGS assays spanning both left and right homology arms. Editing efficiency was measured by read counts of edits specific to wild-type specific sequences. Untreated human DNA mixed with PAH-032h vector served as a negative control.

From these studies, it can be concluded that: (1) blood Phe concentration is reduced to a therapeutically relevant threshold (≤360 μM) following administration of the mouse construct PAH-006m packaged in AAVHSC15 capsid into PAH^{enu2} mice; (2) similar levels of mRNA expression were observed across species between the mouse-specific construct PAH-006m in PAH^{enu2} mice and the human specific construct PAH-032h in the human liver tissue of FRGR mice, both packaged in AAVHSC15 capsid; (3) no significant target insertion was observed in mouse hepatocytes (using mouse-specific assays) following administration of the human specific construct PAH-032h packaged in AAVHSC15 capsid into FRG® mice, demonstrating the sequence/species specificity of the human homology arms; and (4) similar levels of on-target integration per allele were achieved across species between the mouse-specific construct PAH-006m in the PAH^{enu2} mouse (4-7%) and the human specific construct PAH-032h in the FRG mouse (˜6%).

### Example 3: In Vivo Efficacy of PAH-006m-LP-1 in PAH^{enu2} Mice

The ability of the PAH-006m-LP-1 vector to reduce blood Phe levels in mice was assessed and correlated with hPAH mRNA expression and target insertion at the mouse PAH locus. Specifically, four PAH^{enu2} mice received single intravenous administration of the murine specific PAH-006m-LP-1 vector packaged in AAVHSC15 capsid covering a ˜2-log dose range, with dose levels of 5E+12, 1E+13, 1E+14, and 2E+14 vg/kg (“GE 5E12,” “GE 1E13,” “GE 1E14,” and “GE 2E14,” respectively). In addition, a control arm containing an AAV gene transfer construct expressing hPAH but with no homology arms was administered at a dose of 1E+13 vg/kg (“GT (+) control 1E13”). Four PAH^{enu2} mice were also administered formulation buffer as a negative control (“FB”). Blood Phe concentration was measured over the course of the experiment.

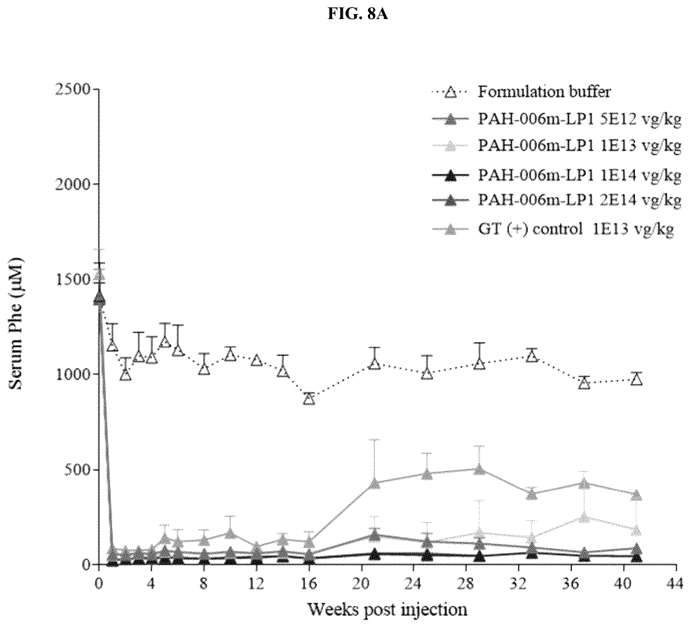

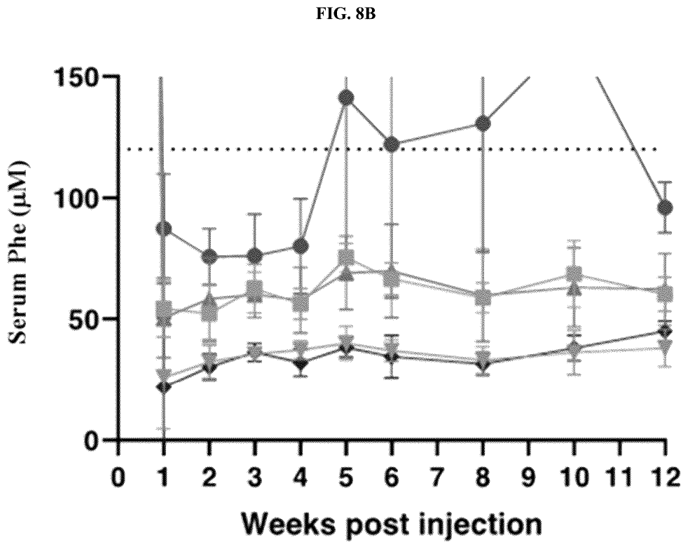

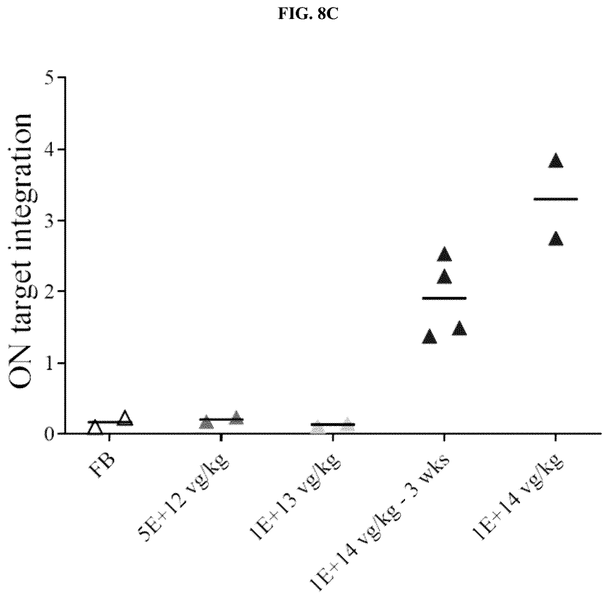

A subset of animals dosed at 1E+14 vg/kg were sacrificed at the 2-week timepoint to measure the level of target gene insertion in the liver. A level of 5±2% target gene insertion per allele was detected. The blood Phe concentrations in the mice through week 41 are shown in FIG. 8A. FIG. 8A shows the full data set, while FIG. 8B shows the data out to 12 weeks with a reduced y-axis scale to allow differences between the dose levels to be seen more clearly. Blood Phe concentration was reduced to normal levels by one week following administration of all dose levels, with the 1E+14 and 2E+14 doses resulting in the lowest levels.

On-target analysis was performed on animals dosed at 5E12 vg/kg (“5E+12”), 1E13 vg/kg (“1E+13”), and 1E14 vg/kg (“1E+14”), as indicated (FIG. 8C). On-target integration was measured by next generation sequencing as described in Example 2. Integration rates were calculated based on the number of sequences with the integration divided by total number of sequences (with or without the integration event). In FIG. 8C, all data was collected at 43 weeks post-dosing, except “1E+14 vg/kg-3 wks” where the data was collected at 3 weeks post-dosing. As shown, on-target integration was detected in animals dosed with 1E14 vg/kg PAH-006m-LP-1 vector packaged in AAVHSC15 capsid at both 3 and 43 weeks post-dosing.

### Example 4: In Vitro Editing Specificity of PAH-032h in Non-Human Primate and Human Hepatocytes

Cynomolgus monkeys exhibit 94.2% sequence identity at the PAH locus to the human homology arm sequences of PAH-032h. To determine the specificity of PAH-032h for the human PAH locus, the ability of PAH-032h to edit the PAH locus in a mixture of primary cynomolgus monkey and primary human hepatocytes was assessed. A total of 5E+5 hepatocytes from a single cynomolgus monkey and a single human donor were treated with 1.5E+5 vg/cell of PAH-032h vector packaged in an AAVHSC15 capsid. Editing of the PAH locus was assessed by PCR using primers specific for the human edited allele and primers specific for the non-human primate edited allele. These PCR primers were designed to show either presence or absence of integration and were not quantitative. Accordingly, measurements of PCR efficiency were determined in positive control DNA across a standard curve of dilutions. This PCR assay had a lower limit of detection of 15 copies of edited allele (2.618E-5 amol).

Targeted integration of the payload into the PAH locus of human hepatocytes was observed. In contrast, no integration of the payload into the homologous cynomolgus monkey loci was detected. These results indicate that the human homology arms were specific for the human PAH locus in these assays.

### Example 5: Comparison Between Gene Transfer/Gene Editing Vector and Episomal Transgene Expression Vector

To investigate the dose response of integrated transgene expression and episomal transgene expression, a head-to-head comparison was performed using the mouse-specific gene transfer/gene editing AAV vector, PAH-006m-LP-1, and an episomal transgene expression AAV vector (containing the same transcriptional regulatory element as PAH-006m-LP-1, but lacking homology arms), packaged in AAVHSC15 capsid. The only difference between the payloads of the two vectors was the presence of two silent nucleotide changes in the PAH coding sequence of PAH-006m-LP-1. Five male, 4-week-old PAH^{enu2} mice were intravenously administered 1E12 vg/kg (“1E+12”), 5E12 vg/kg (“5E+12”), 1E13 vg/kg (“1E+13”), 5E13 vg/kg (“5E+13”), or 1E14 vg/kg (“1E+14”) doses of either the mouse-specific gene transfer/gene editing vector or the episomal transgene expression vector, in each case packaged in AAVHSC15, or a formulation buffer control (FB). For the episomal transgene expression vector packaged in AAVHSC15, after an official titering procedure, the titer of the lot used was accordingly adjusted to 7.8E11 vg/kg (“7.8E+11”), 3.9E12 vg/kg (“3.9E+12 vg/kg”), 7.8E12 vg/kg (“7.8E+12”), 3.9E13 vg/kg (“3.9E+13”), and 7.8E13 vg/kg (“7.8E+13”). It was found that targeted integration in mice dosed with PAH-006m-LP-1 was detectible and stable. A dose response between phenotype correction and levels of integration was also found.

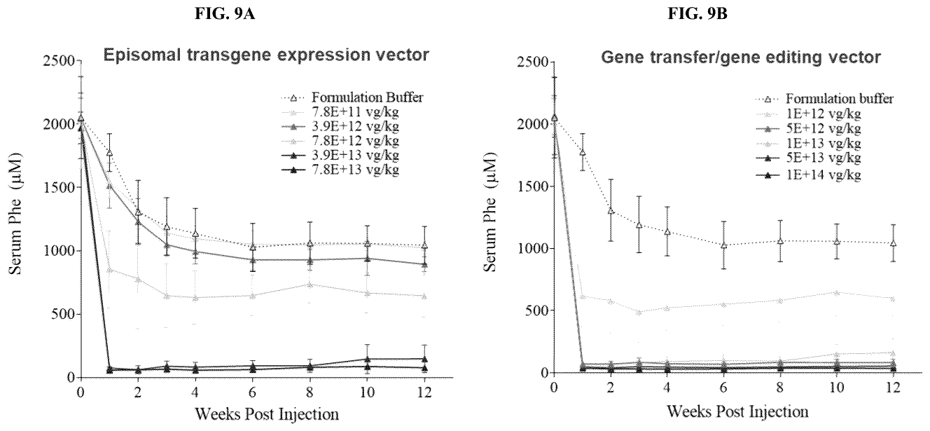

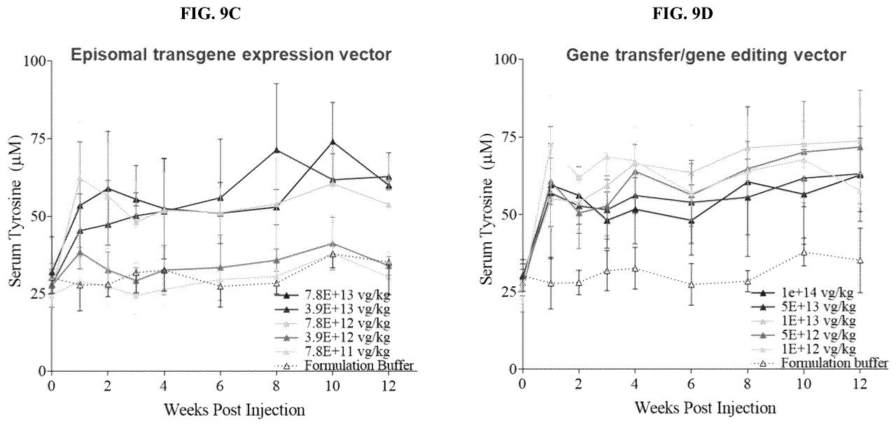

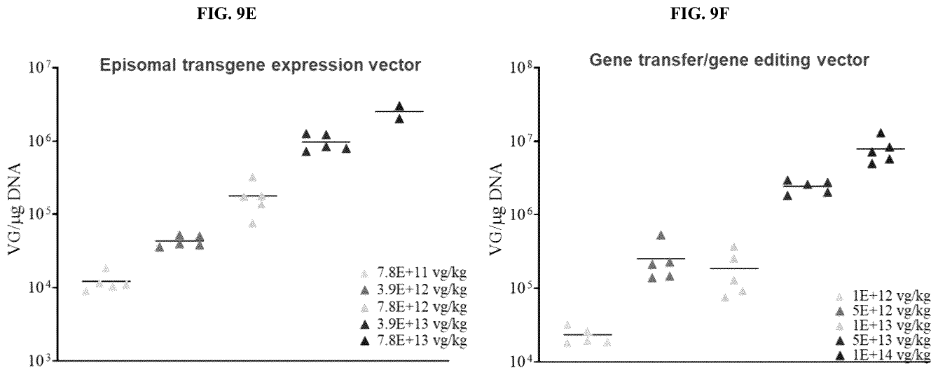

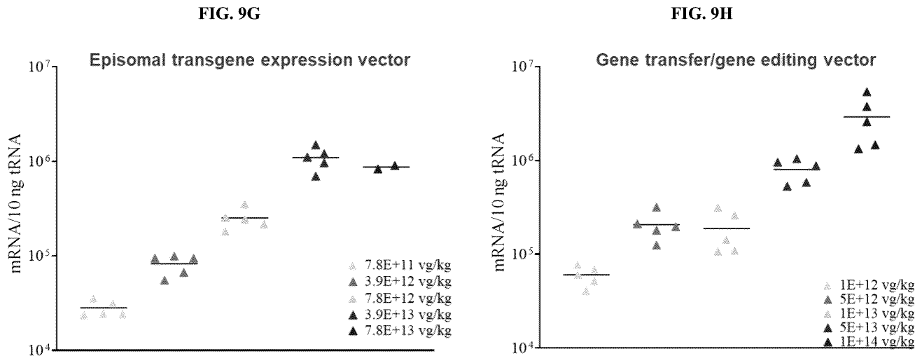

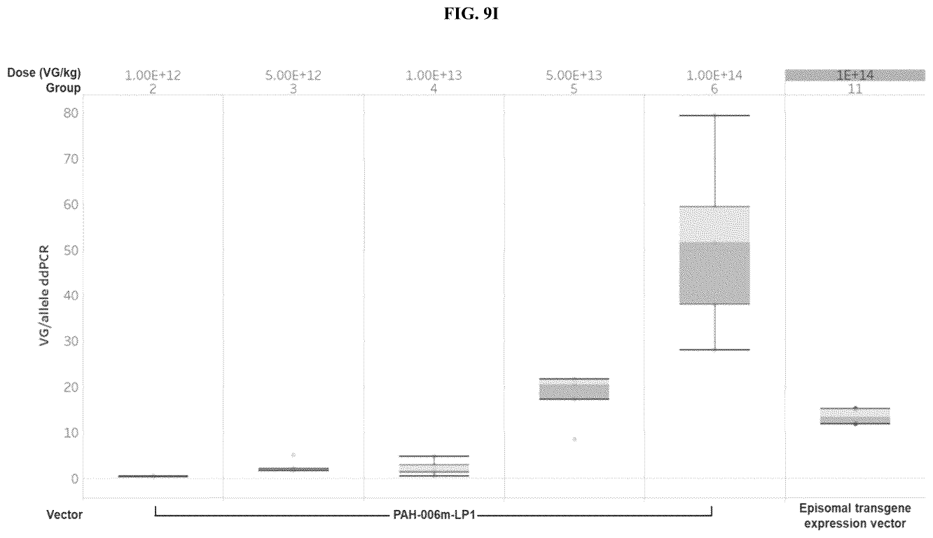

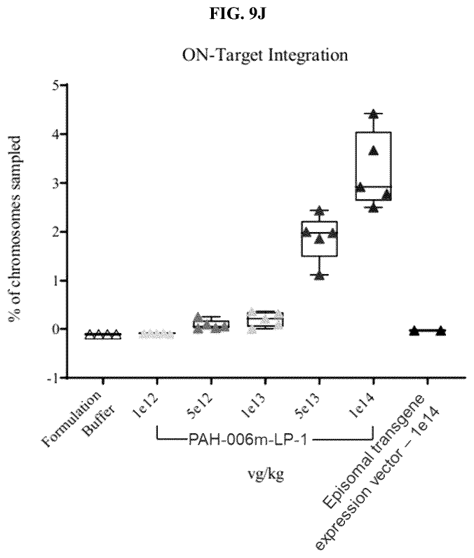

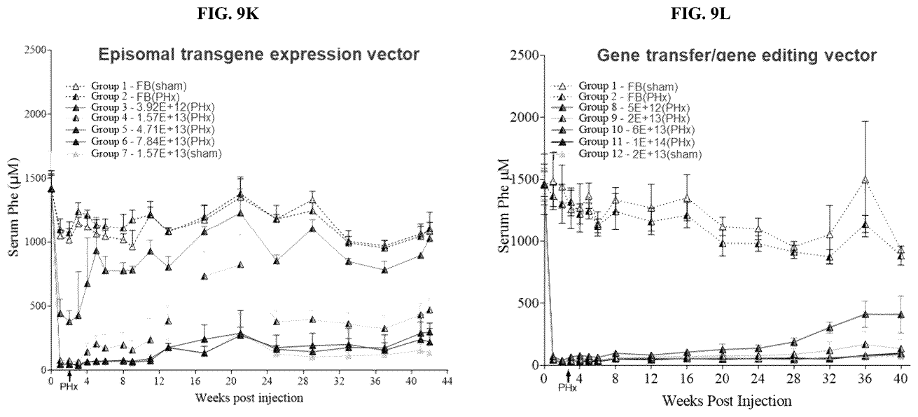

FIGS. 9A and 9B show 12-week time courses of the serum Phe levels in the mice that were dosed as described above. As shown, a reduction in serum Phe was observed in mice that received the episomal transgene expression vector packaged in AAVHSC15 capsid (FIG. 9A), or the gene transfer/gene editing vector packaged in AAVHSC15 capsid (FIG. 9B). Corresponding increases in blood Tyr concentration, a downstream metabolite of Phe, were also observed in mice that received the episomal transgene expression vector packaged in AAVHSC15 capsid (FIG. 9C), or the gene transfer/gene editing vector packaged in AAVHSC15 capsid (FIG. 9D).

FIGS. 9E and 9F show the vector genome levels in PAH^{enu2} mice dosed with the episomal transgene expression vector (FIG. 9E) or PAH-006m-LP1 (FIG. 9F), in each case packaged in AAVHSC15 capsid, at the indicated doses. Vector genomes per ug of DNA were measured using quantitative PCR using a coding sequence specific primer and probes relative to input genomic DNA target. A dose response in both vector genome copy number and mRNA was observed for both PAH-006m-LP-1 and the episomal transgene expression vector. The vector copy number for PAH-006m-LP-1 (FIG. 9F) was higher across all doses compared to the episomal transgene expression vector (FIG. 9E). Differences in VG/ug between the transgene and the integration vector are observed and may reflect differences in stability between self-complementary and single-stranded vectors.

FIGS. 9G and 9H show the PAH transgene mRNA expression levels in PAH^{enu2} mice dosed with the episomal transgene expression vector (FIG. 9G) or PAH-006m-LP1 (FIG. 9H), in each case packaged in AAVHSC15 capsid, at the indicated doses. mRNA levels were measured by quantitative RT-PCR using coding sequence specific primers and probes relative to total RNA. As shown, mRNA expression was found to be dose-responsive, and expression level was found to be slightly higher in mice that received PAH-006m-LP-1 packaged in AAVHSC15 capsid as compared to mice that received the episomal transgene expression vector packaged in AAVHSC15 capsid.

To assess the quantity and fidelity of target locus integration in PAH^{enu2} mice, two methods were performed to detect integration events: ddPCR and next generation sequencing.

The level of on-target integration at the PAH locus was measured using the ddPCR linkage assay as described in Example 2. FIG. 9I shows the amount of on-target integration (measured in viral genomes per PAH allele detected by ddPCR) at 12 weeks post-dosing for the various doses of PAH-006m-LP1 and episomal transgene expression vector control indicated. As shown, a dose response of targeted integration detected by ddPCR was observed in mice that received PAH-006m-LP1 packaged in AAVHSC15 capsid (FIG. 9I; Groups 2, 3, 4, 5, and 6). In comparison, no targeted integration was detected by ddPCR in mice that received the episomal transgene expression vector packaged in AAVHSC15 capsid (FIG. 9I; Group 11).

FIG. 9J shows the frequency of on-target vector insertion detected (using the NGS approach described in Example 2) 12 weeks post-dosing with the indicated doses of PAH-006m-LP1 packaged in AAVHSC15. As shown, a dose response of targeted integration detected by NGS was observed in mice that received PAH-006m-LP1 packaged in AAVHSC15 capsid. In comparison, no targeted integration was detected by NGS in mice that received the episomal transgene expression vector packaged in AAVHSC15 capsid. Integration level was found to be dose-responsive and higher than 2% at doses greater than 5E13 vg/kg.

To evaluate the effect of integrated transgene expression and episomal transgene expression in proliferating cell populations, 10-week-old PAH^{enu2} mice were intravenously administered 5E12 vg/kg, 2E13 vg/kg, 6E13 vg/kg, or 1E15 vg/kg doses of either the mouse-specific gene transfer/gene editing vector or the episomal transgene expression vector, in each case packaged in AAVHSC15, or a formulation buffer control (FB). For the episomal transgene expression vector packaged in AAVHSC15, after an official titering procedure, the titer of the lot used was accordingly adjusted to 3.92E12 vg/kg (“3.92E+12”), 1.57E13 vg/kg (“1.57E+13 vg/kg”), 4.71E13 vg/kg (“4.71E+13”), and 7.84E13 vg/kg (“7.84E+13”). About 2 weeks post-dosing, the mice underwent either a 70% partial hepatectomy (PHx) to induce rapid hepatocyte cycling, or a sham surgery (sham). FIGS. 9K and 9L show a time course out to 42 weeks (FIG. 9K) or 40 weeks (FIG. 9L) of the serum Phe levels in these mice. As shown, in mice treated with the episomal transgene expression vector (FIG. 9K), serum Phe levels were reduced at weeks 1 and 2. Following hepatectomy at the 2-week timepoint, the Phe levels at the 5E12 vg/kg and 2E13 vg/kg dose levels increased during the period of accelerated liver regeneration. The sham surgery animals treated with 2E13 vg/kg episomal transgene expression vector did not show a loss of this response, indicating that the increase in serum Phe levels in the episomal transgene expression vector treated hepatectomized mice may be due to vector dilution under conditions of accelerated liver growth that occurred following hepatectomy. Mice treated with the gene transfer/gene editing vector (FIG. 9L) showed a stable reduction in Phe levels throughout the period of accelerated liver growth in the partially hepatectomized mice. This result may be indicative of the benefit of integration at the target mouse Pah locus.

### Example 6: Comparison Between Mouse-Specific Vector and Human-Specific Vector

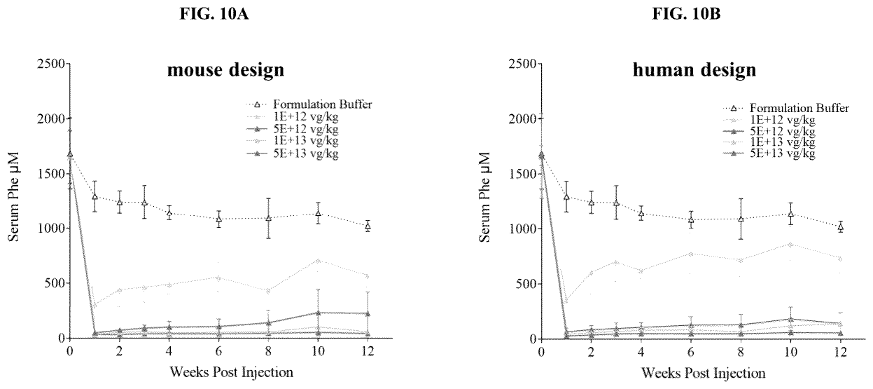

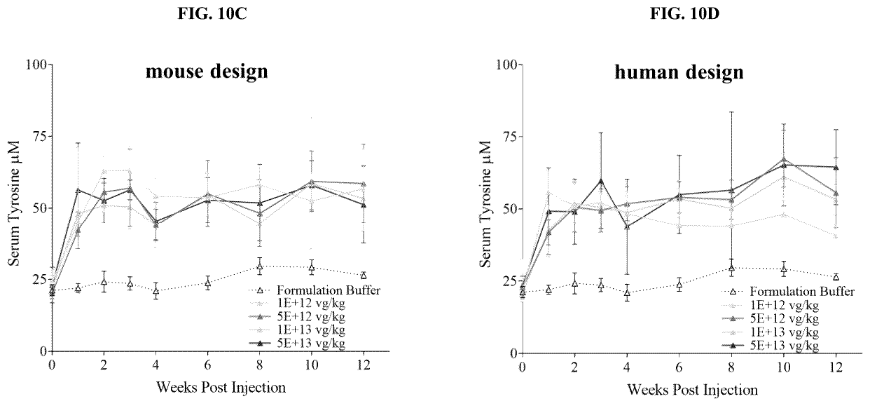

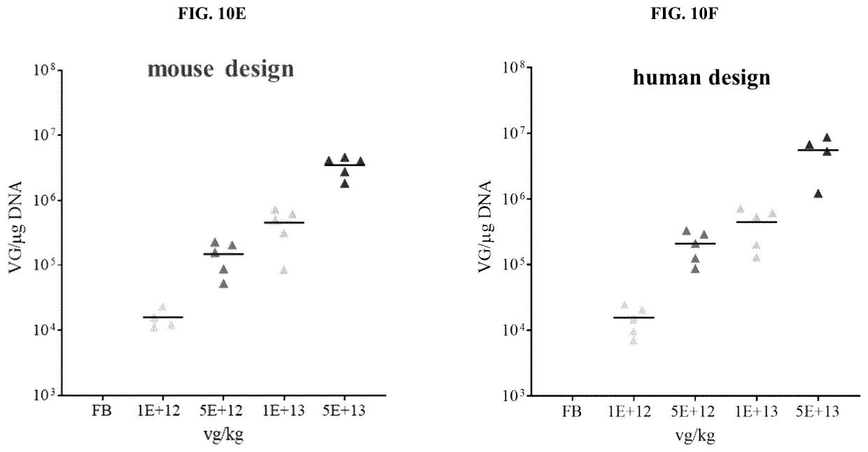

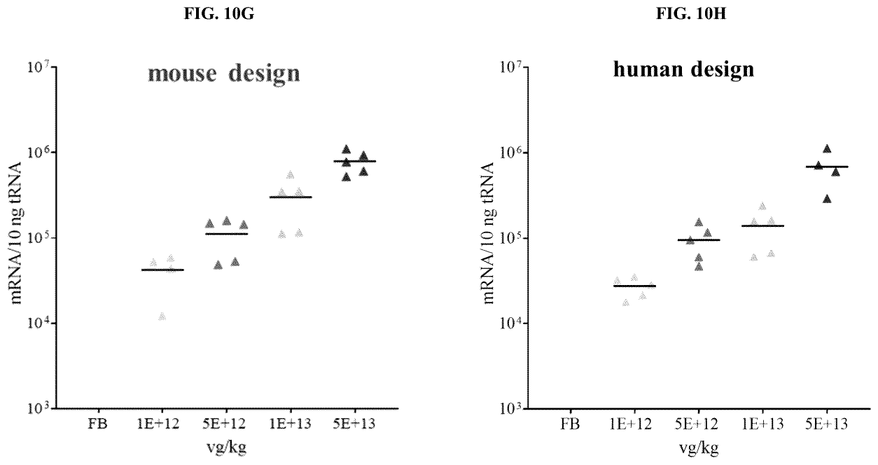

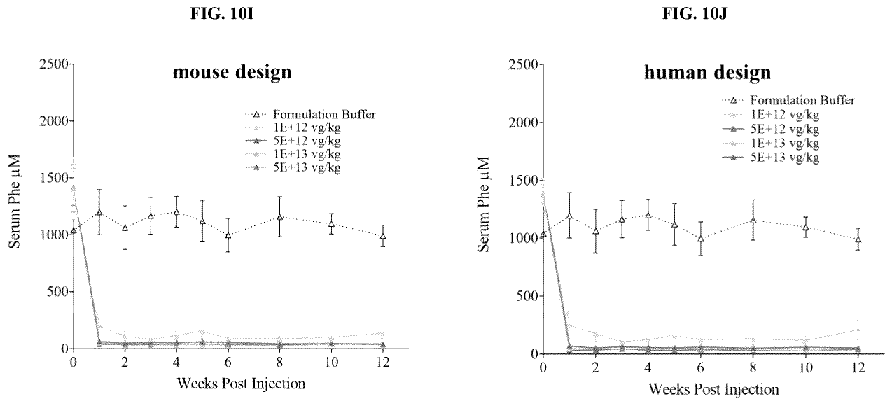

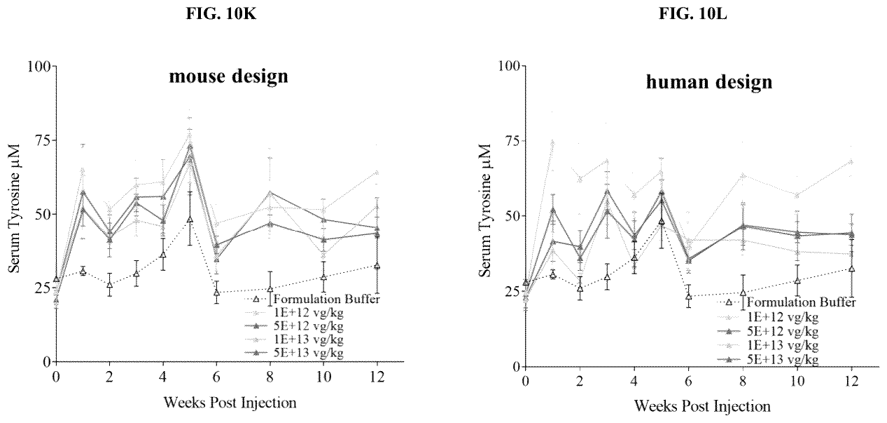

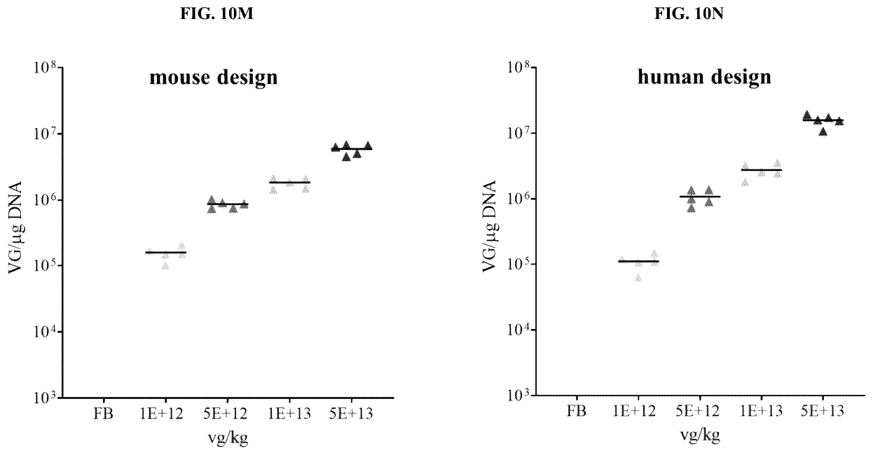

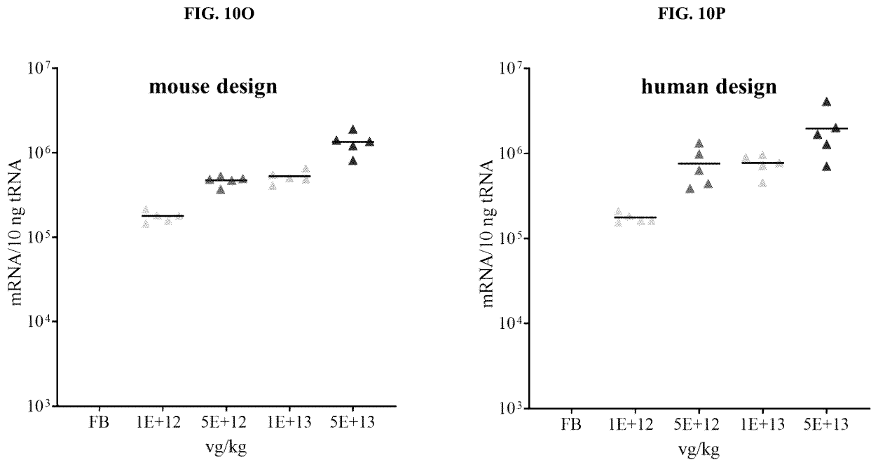

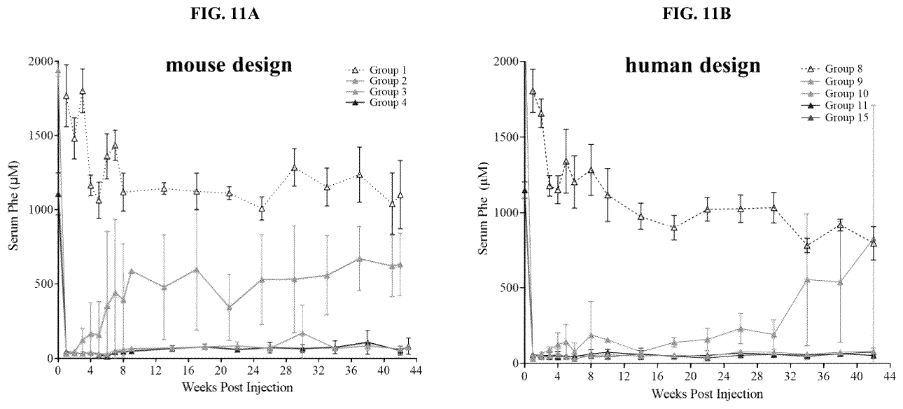

To investigate dose range differences between the mouse-specific gene transfer/gene editing AAV vector (PAH-006m-LP-1; “mouse design”), and the human-specific gene transfer/gene editing AAV vector (hPAH-hI1C-032-LP1-SD3; “human design”) was used in a head-to-head study. Four to five male, 4-week-old (FIGS. 10A-10H) or 10-week-old (FIGS. 10I-10P) PAH^{enu2} mice were intravenously administered 1E12 vg/kg, 5E12 vg/kg, 1E13 vg/kg, or 5E13 vg/kg of either the mouse design vector (FIGS. 10A, 10C, 10E, 10G, 10I, 10K, 10M, and 10O) or the human design vector (FIGS. 10B, 10D, 10F, 10H, 10J, 10L, 10N, and 10P), in each case packaged in AAVHSC15, or a formulation buffer control (FB).

The human design vector comprises human homology arm sequences and would not be expected to integrate into the mouse genome via homologous recombination due to sequence differences, accordingly the human design vector served as an episomal-only control in these experiments. As shown, 4-week-old mice dosed with the human design vector packaged in AAVHSC15 capsid (FIG. 10B) exhibited an improvement in serum Phe reduction, similar to mice administered the mouse design vector (FIG. 10A). A corresponding increase in blood Tyr concentration was also observed in the 4-week-old mice dosed with the human design vector packaged in AAVHSC15 capsid (FIG. 10D) and in the mice dosed with the mouse design vector (FIG. 10C). As shown in FIGS. 10E and 10F, a dose response was observed for vector genome levels detected in the liver of 4-week-old mice dosed with the human design vector (FIG. 10F) or the mouse design vector (FIG. 10E) packaged in AAVHSC15 capsid. A dose response was also observed for mRNA levels detected in the liver of 4-week-old mice dosed with the human design vector (FIG. 10H) or the mouse design vector (FIG. 10G) packaged in AAVHSC15 capsid.

At 10 weeks old, mice dosed with the human design vector packaged in AAVHSC15 capsid (FIG. 10I) were found to have a similar reduction in serum Phe levels, compared to mice dosed with the mouse design vector (FIG. 10H). A corresponding increase in blood Tyr concentration was also observed in the 10-week-old mice dosed with the human design vector packaged in AAVHSC15 capsid (FIG. 10K) and in mice dosed with the mouse design vector (FIG. 10J). As shown in FIGS. 10M and 10N, a dose response was observed for vector genome levels detected in the liver of 10-week-old mice dosed with the human design vector (FIG. 10N) or the mouse design vector (FIG. 10M) packaged in AAVHSC15 capsid. A dose response was also observed for mRNA levels detected in the liver of 10-week-old mice dosed with the human design vector (FIG. 10P) or the mouse design vector (FIG. 10O) packaged in AAVHSC15 capsid.

### Example 7: Effect of a Single Dose of Mouse-Specific Vector or Human-Specific Vector

To evaluate the effect of the age of PAH^{enu2} mice on the response to a single dose of the mouse-specific gene transfer/gene editing AAV vector (PAH-006m-LP-1; “mouse design”) or the human-specific gene transfer/gene editing AAV vector (hPAH-hI1C-032-LP1-SD3; “human design”), mice at 2-, 4-, and 10-weeks of age were selected. This allowed for a comparison between the effect of pediatric liver growth (2- and 4-week-old mice) and the liver of an adult (10-week-old mice). A single 1E14 vg/kg dose of the mouse design vector packaged in AAVHSC15 or the human design vector packaged in AAVHSC15 was intravenously administered to PAH^{enu2} mice at 2-, 4-, or 10-weeks of age. A total of four to five male mice in each age group were administered the respective packaged vectors. Formulation buffer was administered as control. As the human design vector does not integrate into the mouse Pah locus, it provides a control to demonstrate the effect of episomal expression on mice of different ages. In this example, after an official titering procedure, the titer of the lot used was accordingly adjusted to 9.07E13 vg/kg for the mouse design and 1.06E14 vg/kg for the human design. The various dosing cohorts studied are set forth in Table 2.

TABLE 2
Dosing Cohorts

| | | Dose | Number of mice | Necropsy |
|---|---|---|---|---|
| | | Level | dosed/Sex (age at | (weeks post- |
| Group | Test Article | (vg/kg) | dosing) | administration) |
| 1 | Formulation | NA | 5/M (2-weeks) | 43 weeks |
| | buffer | | | |
| 2 | Mouse design | 1E + 14 | 5/M (2-weeks) | |
| 3 | vector packaged | (9.07E13) | 4/M (4-weeks) | |
| 4 | in AAVHSC15 | | 4/M (10-weeks) | |
| 5 | | | 4/M (2-weeks) | 2 weeks |
| 6 | | | 4/M (2-weeks) | 4 weeks |
| 7 | | | 4/M (2-weeks) | 8 weeks |
| 8 | Formulation | NA | 4/M (2-weeks) | 42 weeks |
| | buffer | | | |
| 9 | Human design | 1E + 14 | 4/M (2-weeks) | |
| 10 | vector packaged | (1.06E14) | 4/M (4-weeks) | |
| 11 | in AAVHSC15 | | 4/M (10-weeks) | |
| 12 | | | 4/M (2-weeks) | 2 weeks |
| 13 | | | 4/M (2-weeks) | 4 weeks |
| 14 | | | 4/M (2-weeks) | 8 weeks |
| 15 | Mouse design | | 4/M (4-weeks) | 42 weeks |
| | vector packaged | | | |
| | in AAVHSC15 | | | |

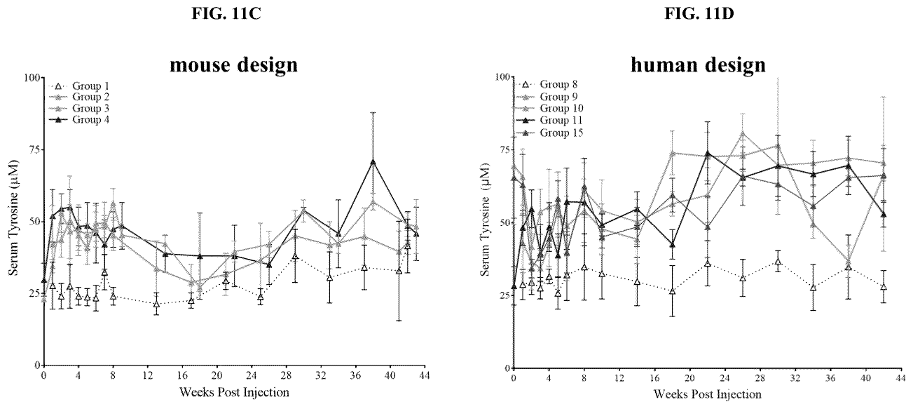

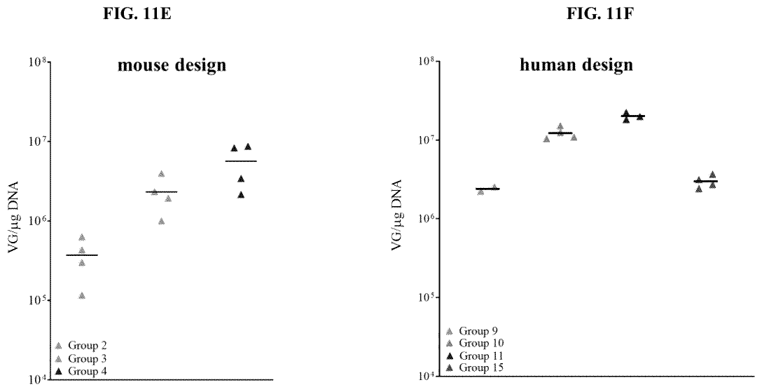

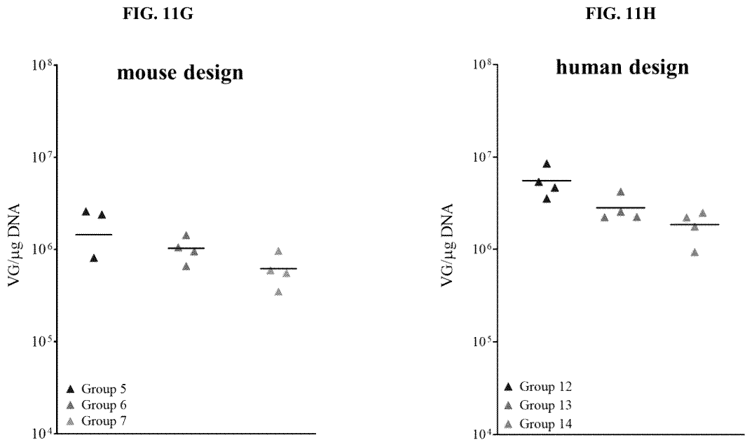

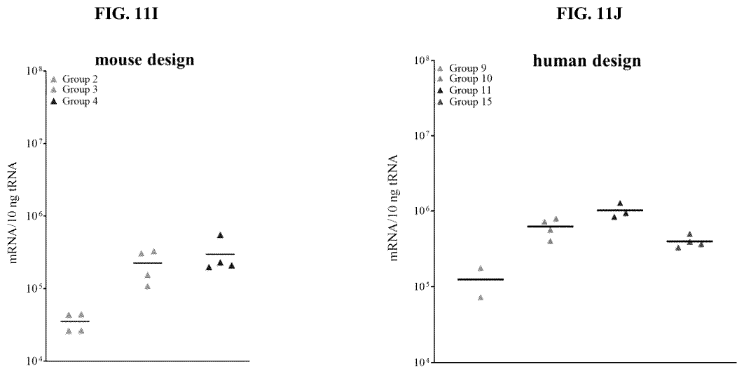

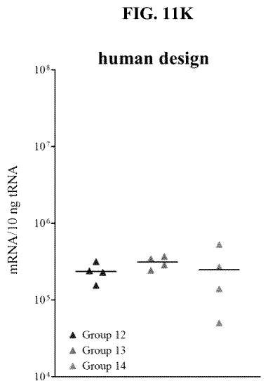

As set forth in Table 2, Groups 1˜4 were sacrificed 43 weeks post-administration, and Groups 8-11, and 15 were sacrificed 42 weeks post-administration. Serum Phe and Tyr levels were examined over time for Groups 1-4, 8-11, and 15 (FIGS. 11A-11D). FIGS. 11E-11K show the vector genome levels and mRNA levels detected in the livers of mice at the necropsy time points indicated in Table 2. mRNA levels in the livers of Groups 5-7 mice were not measured.

In mice treated with the mouse design vector, serum Phe levels were reduced to normal levels within one week following administration of the vector (FIG. 11A). For the treated 4- and 10-week-old mice (Groups 3 and 4), the reduction in Phe levels was maintained through 43 weeks, demonstrating long term durability of response to treatment. For the 2-week-old mice, the results were variable. A corresponding increase in serum Tyr concentration was observed in the 4- and 10-week-old mice through 43 weeks (FIG. 11C). FIG. 11E shows the vector genome levels detected in the liver of 2-, 4-, and 10-week-old mice treated with the mouse design vector. FIG. 11I shows the mRNA levels detected in the liver of 2-, 4-, and 10-week-old mice treated with the mouse design vector.

In mice treated with the human design vector, serum Phe levels were reduced by the one week time point following administration of the vector (FIG. 11B). For the treated 4- and 10-week-old mice (Groups 10 and 11), the reduction in Phe levels was maintained through 43 weeks, indicating long term durability of response to treatment. In the treated 2-week-old mice (Group 9), Phe levels maintained at clinically relevant levels of ≤360 μM through 30 weeks. A corresponding increase in serum Tyr concentration was observed in Groups 9 (through 30 weeks), 10 (through 42 weeks), and 11 (through 42 weeks) (FIG. 11D). These data indicate that a 1E14 vg/kg dose of the human design vector packaged in AAVHSC15 is sufficient to provide sustained efficacy through the period of liver growth in a juvenile mouse, and that the effect is sustained for at least 30 weeks post-administration. FIG. 11F shows the vector genome levels detected in the liver of 2-, 4-, and 10-week-old mice treated with the human design vector. FIG. 11J shows the mRNA levels detected in the liver of 2-, 4-, and 10-week-old mice treated with the human design vector.

FIGS. 11G and 11H show that in 2-week-old mice treated with the mouse design vector (FIG. 11G; Groups 5-7) and 2-week-old mice treated with the human design vector (FIG. 11H; Groups 12-14), vector genome levels were sustained up to 8 weeks post-administration. FIG. 11K shows that mRNA levels of 2-week-old mice treated with the human design vector were sustained up to 8 weeks post-administration.

### Example 8: Kinetics and Durability of Integration in Adult PAH^{enu2} Mice

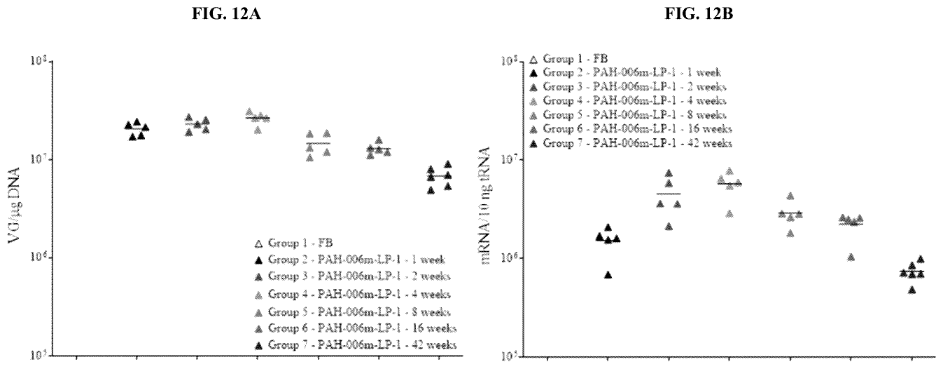

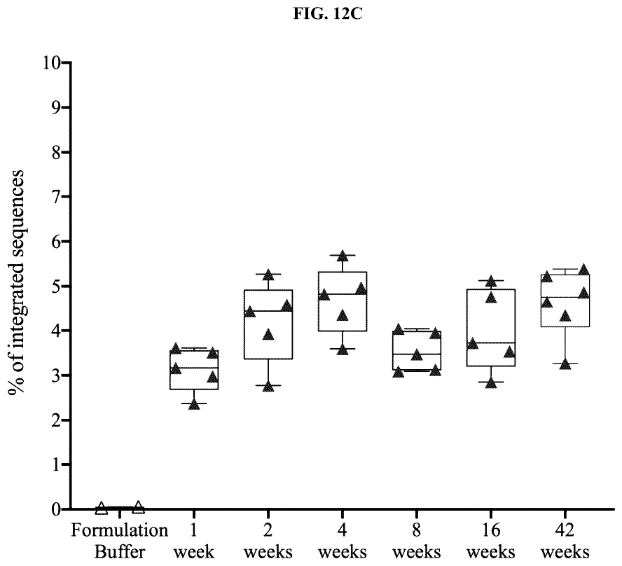

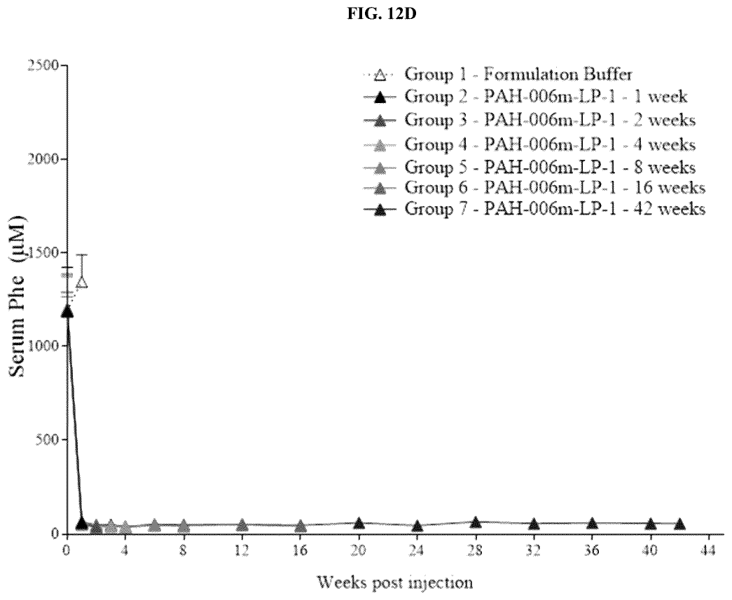

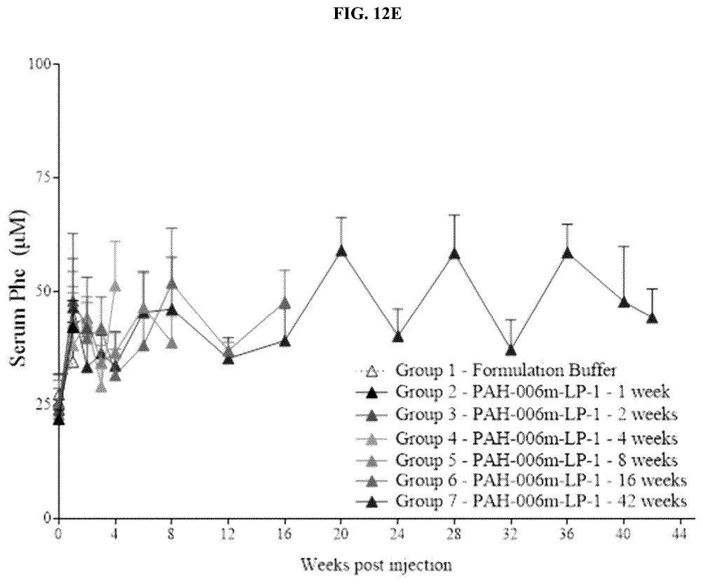

To determine kinetics and durability of vector integration over time, 10-week-old male PAH^{enu2} mice were administered a mouse-specific gene transfer/gene editing AAV vector (PAH-006m-LP-1) packaged in AAVHSC15 capsid at a dose of 1E14 vg/kg. Six cohorts were sacrificed at 1, 2, 4, 8, and 16 weeks post-dosing (five mice per cohort). The kinetics of integration was investigated by monitoring serum Phe levels over time, vector genome levels over time, PAH transgene mRNA expression levels over time, and targeted integration levels over time, at the various time points. It was found that vector genome levels, PAH transgene mRNA expression levels, and integration frequencies were stable over time (FIGS. 12A-12C).

FIGS. 12A and 12B show a time course of vector genome levels (FIG. 12A) and PAH transgene mRNA expression levels (FIG. 12B) in PAH^{enu2} mice administered PAH-006m-LP-1 packaged in AAVHSC15 capsid at a dose of 1E14 vg/kg. The vector genome and the mRNA levels over time were found to be stable up to 42 weeks. Vector genome levels were measured using quantitative PCR using a coding sequence specific primer and probes relative to input genomic DNA target. Measurement of expression was performed by quantitative RT-PCR using coding sequence specific primers and probes relative to total RNA.

A long-read next generation sequencing (NGS) method was developed to quantitate integrated versus unintegrated alleles. Long-read sequencing was selected due to the length of the homology arms requiring a read through of the integration sequence and into the genomic DNA. The method was able to distinguish between integrated alleles, wild-type alleles, and vector genomes that are present in a given sample. PCR is performed using three primers specific to i) the genomic region; ii) a region specific to the integrated allele; and iii) a region specific to the wild-type allele. The frequency of on-target vector integration was determined by competitively amplifying both wild-type and integrated alleles at the PAH region using DNA derived from the livers of treated animals. The long-read sequencing of the amplified products covered the homology arm, which was identical across genomic, integrated, and vector genome sequences. The adjoining sequences, which were also covered by long-read sequencing, determine whether the source of the read was from wild-type, integrated, or vector genomes. Contiguous sequences that included both the genomic DNA and the silently altered hPAH transgene were tallied as integrated alleles, while sequences that included only the genomic DNA without the silently altered hPAH transgene in the target integration site were tallied as wild-type alleles. Percentage of integrated sequences were calculated as the number of reads mapped to the integrated reference divided by the total number of reads mapped to the integrated reference, reads mapped to the wild-type reference, and reads mapped to vector genome concatemers. FIG. 12C shows a time course of targeted integration frequency in PAH^{enu2} mice administered PAH-006m-LP-1 packaged in AAVHSC15 capsid at a dose of 1E14 vg/kg. On-target integration was detected at one week post-dosing, achieved peak levels by about 2 weeks, and remained consistent through 42 weeks.

FIGS. 12D and 12E are graphs showing the effect on serum Phe (FIG. 12D) and Tyr (FIG. 12E) in PAH^{enu2} mice administered PAH-006m-LP-1 packaged in AAVHSC15 capsid at a dose of 1E14 vg/kg, up to 42 weeks post-injection.

### Example 9: In Vivo Efficacy of hPAH-hI1C-032-LP1-SD3

To determine kinetics and durability of integration of a human specific gene transfer/gene editing vector over time, male humanized liver FRG mice (“HuLiv” mice; see, Example 2) were administered the human-specific gene transfer/gene editing vector, hPAH-hI1C-032-LP1-SD3, packaged in AAVHSC15 capsid, at a dose of 7E13 vg/kg (Groups 1 and 3) or 2E14 vg/kg (Groups 2 and 4), and sacrificed at 4 (Groups 1 and 2) or 12 weeks (Groups 3 and 4) post-dosing (six male mice per cohort). Control FRG mice were administered formulation buffer and sacrificed at 4 (Group 5) or 12 weeks (Group 6) post-administration.

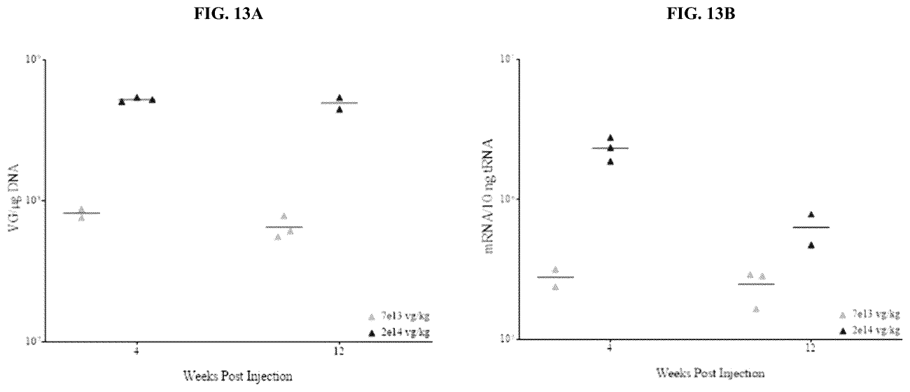

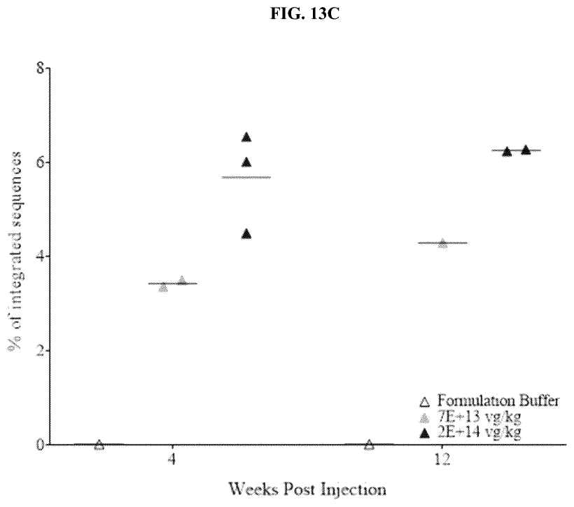

FIGS. 13A and 13B show the vector genome levels and mRNA levels detected in human hepatocytes isolated from HuLiv mice at 4 and 12 weeks after administration of hPAH-hI1C-032-LP1-SD3 packaged in AAVHSC15, at the indicated doses. As shown, the level of vector genomes (FIG. 13A) and mRNA expression (FIG. 13B) in human hepatocytes were found to be dose responsive and stable over time up to 12 weeks. Vector genome levels were measured using quantitative PCR using a coding sequence specific primer and probes relative to input genomic DNA target. Measurement of expression was performed by quantitative RT-PCR using coding sequence specific primers and probes relative to total RNA. FIG. 13C shows the frequency of on-target integration detected in human hepatocytes isolated from HuLiv mice at 4 and 12 weeks after administration of hPAH-hI1C-032-LP1-SD3 packaged in AAVHSC15, at the indicated doses. As shown, the level of on-target integration in human hepatocytes was found to be dose responsive and stable over time up to 12 weeks. Measurement of targeted integration was carried out using the long-read NGS method described in Example 8.

Given that homologous recombination may be driven by short stretches of sequence similarity between the homology arms of the vector and an off-target location in the genome, a PCR-based assay was developed to specifically test for integration into genomic regions that contain sequence similar to the homology arms. The method bioinformatically predicted the most likely regions to undergo off-target homologous recombination in human cells transduced with vector containing homology arms specific to the human PAH locus. These predicted regions were selected based on the highest sequence similarity to the homology arms. A PCR-based method was designed to specifically test transduced samples for any occurrence of off-target integration in the predicted regions. Using this PCR-based method, integration to a level of 1:10000 DNA molecules was able to be detected.

Predicted off-target integration sites were selected based on two criteria: a minimum sequence length of 35 bp; and a minimum sequence identity of 60% relative to the homology arms. Six regions of the genome were identified that met these criteria via bioinformatic alignment against Genome Reference Consortium Human Build 38. PCR primers that allow specific amplification for off-target integration at 5 of these 6 regions were designed. Region 3 was found to be in a highly repetitive region and was dropped from further analysis because specific primers could not be generated. As positive controls (“Pos”), specific control DNAs spiked into genomic DNA, were run for each specific region down to a dilution of 1:10,000, representing 0.01% and the limit of detection of the PCR-based method. For each predicted region, integration-specific primer pairs targeting homologous recombination in either direction (inward (“In”) or outward (“Out”) from the region of homology) were tested independently to query for off-target integration. Off-target integration was defined as positive if a distinct PCR band of the correct size was identified in a sample.

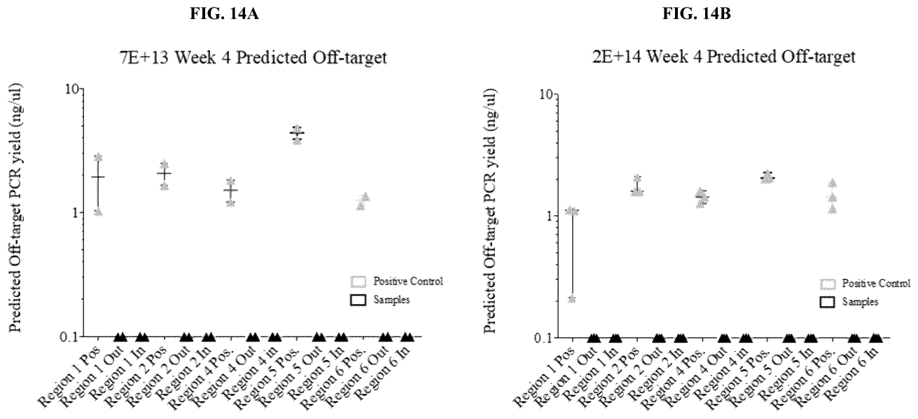

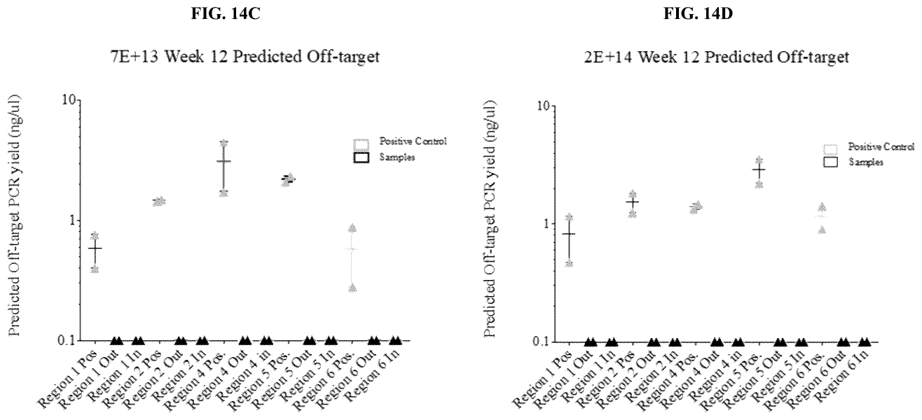

FIGS. 14A-14D show the results of predicted off-target integration determined by the PCR-based method described above, performed on genomic DNA samples isolated from 10-week-old male humanized liver FRG mice administered the human-specific gene transfer/gene editing vector, hPAH-hI1C-032-LP1-SD3, packaged in AAVHSC15 capsid, at a dose of 7E13 vg/kg (FIGS. 14A and 14C) or 2E14 vg/kg (FIGS. 14B and 14D), and sacrificed at 4 (FIGS. 14A and 14B) or 12 weeks (FIGS. 14C and 14D) post-dosing (six male mice per cohort). As shown, no integration was detected at the predicted off-target integration sites.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1-72. (canceled)

73. A method for treating a subject having phenylketonuria (PKU), the method comprising administering to the subject an effective amount of a recombinant adeno-associated virus (rAAV) comprising:
(a) an AAV capsid comprising an AAV capsid protein; and
(b) an rAAV genome comprising: (i) an editing element for editing a target locus in a phenylalanine hydroxylase (PAH) gene, comprising a PAH coding sequence operably linked to a transcriptional regulatory element, wherein the PAH coding sequence comprises the nucleotide sequence set forth in SEQ ID NO: 28; (ii) a 5′ homology arm nucleotide sequence positioned 5′ of the editing element, having homology to a first genomic region 5′ to the target locus; and (iii) a 3′ homology arm nucleotide sequence positioned 3′ of the editing element, having homology to a second genomic region 3′ to the target locus.

74. The method of claim 73, wherein the rAAV is administered intravenously.

75. The method of claim 73, wherein PKU is associated with a PAH gene mutation.

76. The method of claim 73, wherein the subject is a human subject.

77. A packaging system for preparation of an rAAV, wherein the packaging system comprises:
(a) a first nucleotide sequence encoding one or more AAV Rep proteins;
(b) a second nucleotide sequence encoding an AAV capsid protein; and
(c) a third nucleotide sequence comprising an rAAV genome comprising: (i) an editing element for editing a target locus in a phenylalanine hydroxylase (PAH) gene, comprising a PAH coding sequence operably linked to a transcriptional regulatory element, wherein the PAH coding sequence comprises the nucleotide sequence set forth in SEQ ID NO: 28; (ii) a 5′ homology arm nucleotide sequence positioned 5′ of the editing element, having homology to a first genomic region 5′ to the target locus; and (iii) a 3′ homology arm nucleotide sequence positioned 3′ of the editing element, having homology to a second genomic region 3′ to the target locus.

78-82. (canceled)

83. A method for recombinant preparation of an rAAV, the method comprising introducing the packaging system of claim 77 into a cell under conditions whereby the rAAV is produced.

84-86. (canceled)

87. The method of claim 73, wherein the transcriptional regulatory element is capable of mediating transcription in a hepatocyte, a renal cell, a brain cell, a pituitary gland cell, an adrenal gland cell, a pancreatic cell, a urinary bladder cell, a gallbladder cell, a colon cell, a small intestine cell, or a breast cell, optionally wherein:
the transcriptional regulatory element is endogenous to the PAH gene; or
the transcriptional regulatory element is exogenous to the PAH gene, optionally wherein:
the transcriptional regulatory element is liver specific, optionally wherein the transcriptional regulatory element comprises one or more elements selected from the group consisting of a human albumin promoter, a human transthyretin (TTR) promoter, a human ApoE/C-I hepatic control region (HCR) 1 or 2, a human ApoH promoter, a human SERPINA1 (hAAT) promoter, and a hepatic specific regulatory module thereof;
the transcriptional regulatory element comprises a nucleotide sequence at least 90% identical to a sequence selected from the group consisting of SEQ ID NO: 25, 26, 27, and 69;
the transcriptional regulatory element comprises a nucleotide sequence at least 90% identical to the nucleotide sequence set forth in SEQ ID NO: 27;
the transcriptional regulatory element comprises the nucleotide sequence set forth in SEQ ID NO: 27; or
the nucleotide sequence of the transcriptional regulatory element consists of the nucleotide sequence set forth in SEQ ID NO: 27.

88. The method of claim 73, wherein the editing element further comprises an intron element positioned 5′ to the PAH coding sequence and 3′ to the transcriptional regulatory element, optionally wherein the intron element is an exogenous intron element, optionally an SV40 intron element, optionally wherein:
the SV40 intron element comprises a nucleotide sequence at least 90% identical to the nucleotide sequence set forth in SEQ ID NO: 29;
the SV40 intron element comprises the nucleotide sequence set forth in SEQ ID NO: 29; or
the nucleotide sequence of the SV40 intron element consists of the nucleotide sequence set forth in SEQ ID NO: 29.

89. The method of claim 73, wherein the editing element further comprises a polyadenylation sequence 3′ to the PAH coding sequence, optionally wherein the polyadenylation sequence is an exogenous polyadenylation sequence, optionally an SV40 polyadenylation sequence, optionally wherein
the SV40 polyadenylation sequence comprises a nucleotide sequence at least 90% identical to the nucleotide sequence set forth in SEQ ID NO: 31;
the SV40 polyadenylation sequence comprises the nucleotide sequence set forth in SEQ ID NO: 31; or
the nucleotide sequence of the SV40 polyadenylation sequence consists of the nucleotide sequence set forth in SEQ ID NO: 31.

90. The method of claim 73, wherein:
the nucleotide sequence 5′ to the target locus is in an intron of a PAH gene, optionally in intron 1 of a PAH gene; and/or
the nucleotide sequence 3′ to the target locus is in an intron of a PAH gene, optionally in intron 1 of a PAH gene.

91. The method of claim 73, wherein the PAH gene is a human PAH gene, optionally wherein the human PAH gene is wild-type or a variant PAH gene.

92. The method of claim 73, wherein the editing element comprises a nucleotide sequence selected from the group consisting of SEQ ID NO: 25, 26, 27, 29, 31, 50, 51, 52, 69, and 70.

93. The method of claim 73, wherein:
the 5′ homology arm nucleotide sequence is at least 90% identical to the first genomic region;
the 3′ homology arm nucleotide sequence is at least 90% identical to the second genomic region;
the first genomic region is located in a first editing window, and the second genomic region is located in a second editing window, optionally wherein:
the first editing window consists of the region of the human genome corresponding to the nucleotide sequence set forth in SEQ ID NO: 37;
the second editing window consists of the region of the human genome corresponding to the nucleotide sequence set forth in SEQ ID NO: 38;
the first genomic region consists of the region of the human genome corresponding to the nucleotide sequence set forth in SEQ ID NO: 37; and/or
the second genomic region consists of the region of the human genome corresponding to the nucleotide sequence set forth in SEQ ID NO: 38.

94. The method of claim 73, wherein:
each of the 5′ and the 3′ homology arm nucleotide sequences independently has a length of about 100 to about 2000 nucleotides;
the 5′ homology arm comprises a nucleotide sequence that is at least 90% identical to the nucleotide sequence set forth in SEQ ID NO: 23;
the nucleotide sequence of the 5′ homology arm consists of the nucleotide sequence set forth in SEQ ID NO: 23;
the 3′ homology arm comprises a nucleotide sequence that is at least 90% identical to the nucleotide sequence set forth in SEQ ID NO: 24; and/or
the nucleotide sequence of the 3′ homology arm consists of the nucleotide sequence set forth in SEQ ID NO: 24.

95. The method of claim 73, wherein the rAAV genome comprises a nucleotide sequence that is at least 85% identical to the nucleotide sequence set forth in SEQ ID NO: 43.

96. The method of claim 73, wherein the rAAV genome further comprises a 5′ inverted terminal repeat (5′ ITR) nucleotide sequence 5′ of the 5′ homology arm nucleotide sequence, and a 3′ inverted terminal repeat (3′ ITR) nucleotide sequence 3′ of the 3′ homology arm nucleotide sequence, optionally wherein:
the 5′ ITR nucleotide sequence is at least 85% identical to the nucleotide sequence set forth in SEQ ID NO: 14, and/or the 3′ ITR nucleotide sequence is at least 85% identical to the nucleotide sequence set forth in SEQ ID NO: 18; and/or
the rAAV genome comprises the nucleotide sequence set forth in SEQ ID NO: 73, 74, 75, or 76.

97. The method of claim 73, wherein the rAAV genome comprises a nucleotide sequence that is at least 85% identical to the nucleotide sequence set forth in SEQ ID NO: 45.

98. The method of claim 73, wherein:
the AAV capsid protein comprises an amino acid sequence that is at least 95% identical to the amino acid sequence of amino acids 203-736 of SEQ ID NO: 1, 2, 3, 4, 5, 6, 7, 8, 9, 10, 11, 12, 13, 15, 16, or 17, optionally wherein:
(i) the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G;
(ii) (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C; and/or
(iii) the capsid protein comprises the amino acid sequence of amino acids 203-736 of SEQ ID NO: 1, 2, 3, 4, 5, 6, 7, 8, 9, 10, 11, 12, 13, 15, 16, or 17;
the AAV capsid protein comprises an amino acid sequence that is at least 95% identical to the amino acid sequence of amino acids 138-736 of SEQ ID NO: 1, 2, 3, 4, 5, 6, 7, 8, 9, 10, 11, 12, 13, 15, 16, or 17, optionally wherein:
(i) the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G;
(ii) (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C; and/or
(iii) the capsid protein comprises the amino acid sequence of amino acids 138-736 of SEQ ID NO: 1, 2, 3, 4, 5, 6, 7, 9, 10, 11, 12, 13, 15, 16, or 17; and/or
the AAV capsid protein comprises an amino acid sequence that is at least 95% identical to the amino acid sequence of amino acids 1-736 of SEQ ID NO: 1, 2, 3, 4, 5, 6, 7, 8, 9, 10, 11, 12, 13, 15, 16, or 17, optionally wherein:
(i) the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G;
(ii) (a) the amino acid in the capsid protein corresponding to amino acid 2 of SEQ ID NO: 16 is T, and the amino acid in the capsid protein corresponding to amino acid 312 of SEQ ID NO: 16 is Q; (b) the amino acid in the capsid protein corresponding to amino acid 65 of SEQ ID NO: 16 is I, and the amino acid in the capsid protein corresponding to amino acid 626 of SEQ ID NO: 16 is Y; (c) the amino acid in the capsid protein corresponding to amino acid 77 of SEQ ID NO: 16 is R, and the amino acid in the capsid protein corresponding to amino acid 690 of SEQ ID NO: 16 is K; (d) the amino acid in the capsid protein corresponding to amino acid 119 of SEQ ID NO: 16 is L, and the amino acid in the capsid protein corresponding to amino acid 468 of SEQ ID NO: 16 is S; (e) the amino acid in the capsid protein corresponding to amino acid 626 of SEQ ID NO: 16 is G, and the amino acid in the capsid protein corresponding to amino acid 718 of SEQ ID NO: 16 is G; (f) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (g) the amino acid in the capsid protein corresponding to amino acid 505 of SEQ ID NO: 16 is R, and the amino acid in the capsid protein corresponding to amino acid 687 of SEQ ID NO: 16 is R; (h) the amino acid in the capsid protein corresponding to amino acid 346 of SEQ ID NO: 16 is A, and the amino acid in the capsid protein corresponding to amino acid 505 of SEQ ID NO: 16 is R; or (i) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C; and/or
(iii) the capsid protein comprises the amino acid sequence of amino acids 1-736 of SEQ ID NO: 1, 2, 3, 4, 5, 6, 7, 8, 9, 10, 11, 12, 13, 15, 16, or 17.

99. The method of claim 73, wherein the editing element comprises a nucleotide sequence that is at least 85% identical to the nucleotide sequence set forth in SEQ ID NO: 52.

100. The method of claim 73, wherein:
(a) the AAV capsid protein comprises the amino acid sequence of amino acids 203-736 of SEQ ID NO: 16; the amino acid sequence of amino acids 138-736 of SEQ ID NO: 16; and/or the amino acid sequence of amino acids 1-736 of SEQ ID NO: 16; and
(b) the rAAV genome comprises a nucleotide sequence that is at least 85% identical to the nucleotide sequence set forth in SEQ ID NO: 43.

101. The method of claim 73, wherein:
(a) the AAV capsid protein comprises the amino acid sequence of amino acids 203-736 of SEQ ID NO: 16; the amino acid sequence of amino acids 138-736 of SEQ ID NO: 16; and/or the amino acid sequence of amino acids 1-736 of SEQ ID NO: 16; and
(b) the rAAV genome comprises a nucleotide sequence that is at least 85% identical to the nucleotide sequence set forth in SEQ ID NO: 45.
